SEQUENCE LISTING (32 sequences)

SEQ ID NO: 1 - protein, 743 aa, Adeno-associated virus
  VARIANT 1: note = May be absent
  SITE 2: note = Acetylated residue
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSPLNGAVHLYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 606 aa, Adeno-associated virus
  VARIANT 1: note = May be absent
  SITE 2: note = Acetylated residue
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSPLNGAVHLYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 541 aa, Adeno-associated virus
  VARIANT 1: note = May be absent
  SITE 2: note = Acetylated residue
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSPLNGAVHLYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - protein, 743 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - DNA, 145 bp, synthetic construct
  source 1..145: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 6 - DNA, 455 bp, Homo sapiens
  source 1..455: mol_type = genomic DNA
ctgagttgaatatcaacactacaaaccgagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctc

SEQ ID NO: 7 - DNA, 129 bp, Homo sapiens
  source 1..129: mol_type = genomic DNA
agtctgcggtgggcagcggaggagtcgtgtcgtgcctgagagcgcagctgtgctcctgggcaccgcgcagtccgcccccgcggctcctggccagaccacccctaggaccccctgccccaagtcgcagcc

SEQ ID NO: 8 - DNA, 1773 bp, Homo sapiens
  source 1..1773: mol_type = genomic DNA
atgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagatga

SEQ ID NO: 9 - DNA, 202 bp, synthetic construct
  source 1..202: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgc

SEQ ID NO: 10 - DNA, 228 bp, synthetic construct
  source 1..228: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 11 - DNA, 1006 bp, synthetic construct
  source 1..1006: mol_type = other DNA
gtaatctcagtaccttgggaggccaaggtggaaggatcactcaaagctaggagctcaagaccaacctgggtaacaaaacaagtccctgttgctacaattagctggtcaaggtggcaagtgcctgtagtcccagctacttgggaggctgaggcgggagggtcacttgagtccagaagttaaaggctacattggagaccctgtctcaaagaaaggaagcagggagggagaattgctgtagctgccaaagatagtgattcctctgttgaatctgggcaaagtgaattgaaaactagacttcagtgctggaagttactgcaggtgtagtggaaatagcaagtgaactagaattagaagtgaacctgaagttgtgactgaattgctgcaattttttgataaaacctgaacagttgaggagttgcttcttgtgagtaagcaaagaaagtggtttcttgagatagaattgactcctggtgaagaactgttgactttagaatattacaaaaacttagttgataaagcagcagcagggtttgagaggattgactccaattttgaaataagttctagtgtgggtaaattgctgtcaaatagtattaattgctacagagacaacttcagtgaaaggaagagtcagtcagcgcagcgcttgtggcaaacttcgtcagtcttattttaagaaattgccacagctaccaccctgatcagtcagcagccaacaacaacgaggcaagatcctctgtcagcaaaaagattttgatttgctgcaggctcacaagattgttagcaattttagcaataaagcaattttaaattaagttatatacaaattattagacaaattgctattgcacacttaatagactttagtgctaacaaaacttttgtaggcactgggaaaccaaaaaattgttgccgcttgctttattgagttggtctggaacctaacctgtagtatctccgaggtttgcctgtatctttaattgtaatttgtccttcacagctagca

SEQ ID NO: 12 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 13 - DNA, 145 bp, synthetic construct; identical to SEQ ID NO: 12
  source 1..145: mol_type = other DNA

SEQ ID NO: 14 - protein, 590 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALR

SEQ ID NO: 15 - DNA, 1782 bp, Homo sapiens
  source 1..1782: mol_type = genomic DNA
atgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtga

SEQ ID NO: 16 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 17 - protein, 17 aa, Homo sapiens
MWTLVSWVALTAGLVAG

SEQ ID NO: 18 - protein, 576 aa, Homo sapiens
TRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 19 - DNA, 3942 bp, synthetic construct
  source 1..3942: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttaccgcgtcaattcgcgcctgagttgaatatcaacactacaaaccgagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagccttcgaattctgcagtcgacgtttaaacgctagcaagcttccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagatgagcggccgcgttaacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcaagcttgtaatctcagtaccttgggaggccaaggtggaaggatcactcaaagctaggagctcaagaccaacctgggtaacaaaacaagtccctgttgctacaattagctggtcaaggtggcaagtgcctgtagtcccagctacttgggaggctgaggcgggagggtcacttgagtccagaagttaaaggctacattggagaccctgtctcaaagaaaggaagcagggagggagaattgctgtagctgccaaagatagtgattcctctgttgaatctgggcaaagtgaattgaaaactagacttcagtgctggaagttactgcaggtgtagtggaaatagcaagtgaactagaattagaagtgaacctgaagttgtgactgaattgctgcaattttttgataaaacctgaacagttgaggagttgcttcttgtgagtaagcaaagaaagtggtttcttgagatagaattgactcctggtgaagaactgttgactttagaatattacaaaaacttagttgataaagcagcagcagggtttgagaggattgactccaattttgaaataagttctagtgtgggtaaattgctgtcaaatagtattaattgctacagagacaacttcagtgaaaggaagagtcagtcagcgcagcgcttgtggcaaacttcgtcagtcttattttaagaaattgccacagctaccaccctgatcagtcagcagccaacaacaacgaggcaagatcctctgtcagcaaaaagattttgatttgctgcaggctcacaagattgttagcaattttagcaataaagcaattttaaattaagttatatacaaattattagacaaattgctattgcacacttaatagactttagtgctaacaaaacttttgtaggcactgggaaaccaaaaaattgttgccgcttgctttattgagttggtctggaacctaacctgtagtatctccgaggtttgcctgtatctttaattgtaatttgtccttcacagctagcagtactaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 20 - DNA, 130 bp, Adeno-associated virus 2
  source 1..130: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 21 - DNA, 130 bp, Adeno-associated virus 2
  source 1..130: mol_type = genomic DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 22 - DNA, 658 bp, synthetic construct
  source 1..658: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 23 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 24 - DNA, 278 bp, synthetic construct
  source 1..278: mol_type = other DNA
tcgaggtgagccccacgttctgcttcactctccccatctcccccccctccccacccccaattttgtatttatttattttttaattattttgtgcagcgatgggggcggggggggggggggggsgcgcgccggggggggggggggggggggggggggggggsggggsgrggcggagaggtgcggcggcagccaatcagagcggcgcgctccgaaagtttccttttatggcgaggcggcggcggcggcggccctataaaaagcgaagcgcgcggcgggcg

SEQ ID NO: 25 - DNA, 196 bp, synthetic construct
  source 1..196: mol_type = other DNA
gcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 26 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 27 - DNA, 135 bp, synthetic construct
  source 1..135: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 28 - DNA, 56 bp, synthetic construct
  source 1..56: mol_type = other DNA
aataaaggaaatttattttcattgcaatagtgtgttggaattttttgtgtctctca

SEQ ID NO: 29 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 30 - DNA, 20 bp, synthetic construct
  source 1..20: mol_type = other DNA
cgtgtgacacgcagaagggt

SEQ ID NO: 31 - DNA, 20 bp, synthetic construct
  source 1..20: mol_type = other DNA
catacactgctgtcccgcgg

SEQ ID NO: 32 - DNA, 20 bp, synthetic construct
  source 1..20: mol_type = other DNA
aggtgagctggggccttctc

(19) United States
(12) Patent Application Publication
(10) Pub. No.: US 2026/0250711 A1
(43) Pub. Date: Aug. 27, 2026

(54) GENE THERAPY FOR FRONTOTEMPORAL DEMENTIA

(71) Applicant: ALEXION PHARMA INTERNATIONAL OPERATIONS LIMITED, Dublin (IE)

(72) Inventors: Robert D. BELL, Boston, MA (US); Ting-Wen CHENG, Boston, MA (US); Jatin NARULA, Boston, MA (US); Clark Qun PAN, Boston, MA (US); David Wayne SOUZA, Boston, MA (US); Ricardos TABET, Boston, MA (US)

(21) Appl. No.: 19/129,198

(22) PCT Filed: Nov. 13, 2023
(86) PCT No.: PCT/IB2023/061448

§ 371 Date: May 12, 2025

Related U.S. Application Data
(60) Provisional application No. 63/383,501, filed on Nov. 13, 2022.
(60) Provisional application No. 63/497,723, filed on Apr. 22, 2023.

(51) Int. Cl. C12N 15/86; A61K 38/17; A61K 48/00; A61P 25/28; C07K 14/47

(52) U.S. Cl. CPC C12N 15/86; A61K 38/1709; A61K 48/005; A61P 25/28; C07K 14/47; C12N 2750/14143; C12N 2750/14151; C12N 2830/48; C12N 2830/50

(57) ABSTRACT

The disclosure describes improved vectors, such as adeno-associated virus (AAV) vectors, for expressing progranulin and variants thereof in transduced cells, and use of such vectors to increase the amount of progranulin in subjects experiencing a progranulin deficiency, such as certain subjects with frontotemporal dementia (FTD) or frontotemporal lobar degeneration (FTLD).

### SEQUENCE LISTING

The instant application contains a Sequence Listing which has been submitted electronically in XML format and is hereby incorporated by reference in its entirety. Said XML copy, created on Nov. 27, 2023, is named FTDPF-100-WO-PCT_SL.xml and is 47,878 bytes in size.

### BACKGROUND

Frontotemporal dementia (FTD) refers to a clinical syndrome characterized by progressively worsening deficits in language, behavior and executive function associated with the selective neurodegeneration of the frontal and temporal cortical lobes (frontotemporal lobar degeneration, or FTLD), as opposed to the more global neurodegeneration commonly seen in Alzheimer's disease and certain other dementias. FTD often strikes its victims in the prime of their lives, with symptom onset most commonly occurring between the ages of 45 and 64. Symptoms then rapidly progress, leading to diminished function and eventually death within an average of 8 years. The only treatments for FTD focus on managing the impact of the inevitable behavioral changes, and none are effective for slowing or reversing the underlying lobar neurodegeneration that is their cause. In view of this overwhelmingly unmet medical need there exists a need in the art for ways of effectively treating or preventing frontotemporal lobar degeneration and the neurological deficits and eventual death to which it leads.

### SUMMARY

To address the need in the art, the present disclosure provides improved adeno-associated virus (AAV) vectors for expressing a human progranulin (PGRN) polypeptide, or variant thereof, methods of producing such AAV vectors, and methods of using such AAV vectors to prevent or treat diseases or disorders in subjects characterized by a deficiency in the amount of human PGRN, or variant thereof, including but not limited to frontotemporal lobar degeneration (FTLD) and frontotemporal dementia (FTD).

Certain enumerated non-limiting embodiments (E) of the disclosure are set forth below. These and related embodiments are described in further detail in the Detailed Description, including the Examples and Drawings. Those skilled in the art will recognize or will be able to ascertain, using no more than routine experimentation, equivalents to the specific embodiments described herein.
E1. A recombinant adeno-associated virus (AAV) vector comprising a nucleotide sequence encoding a human progranulin (PGRN) polypeptide, or variant thereof.
E2. The AAV vector of E1, wherein said PGRN polypeptide variant is a carboxy-terminal truncation variant that lacks one or more amino acids otherwise present in full-length wild-type human PGRN polypeptide, such that the variant PGRN polypeptide has reduced binding to human sortilin receptor (SORT1) protein compared to full-length wild-type human PGRN polypeptide.
E3. The AAV vector of E2, wherein said PGRN polypeptide variant lacks the final three carboxy-terminal amino acids that are present in full-length wild-type human PGRN polypeptide.
E4. The AAV vector of E1 to E3, wherein the amino acid sequence of said PGRN polypeptide variant comprises, consists essentially of, or consists of the amino acid sequence of SEQ ID NO:14 or SEQ ID NO:16.
E5. The AAV vector of E1 to E4, wherein said nucleotide sequence encoding said PGRN polypeptide, or variant thereof, is a wild-type nucleotide sequence.
E6. The AAV vector of E1 to E4, wherein said nucleotide sequence encoding said PGRN polypeptide, or variant thereof, is a codon-optimized nucleotide sequence.
E7. The AAV vector of E6, wherein the codon-optimized nucleotide sequence has a reduced number of CpG di-nucleotides compared to a wild-type nucleotide sequence encoding said PGRN polypeptide, or variant thereof.
E8. The AAV vector of E7, wherein the nucleotide sequence encoding said PGRN polypeptide, or variant thereof, has 1-50, 1-45, 1-40, 1-35, 1-30, 1-25, 1-20, 1-15, 1-10, or 1-5 fewer CpG di-nucleotides compared to a wild-type nucleotide sequence encoding PGRN polypeptide, or variant thereof.
E9. The AAV vector of E7 to E8, wherein said wild-type nucleotide sequence encoding said PGRN polypeptide, or variant thereof, is comprised by the nucleotide sequence of SEQ ID NO: 8 or SEQ ID NO:15.
E10. The AAV vector of E7, wherein the nucleotide sequence encoding said PGRN polypeptide, or variant thereof, is devoid of any CpG di-nucleotides.
E11. The AAV vector of E1 to E10, wherein the nucleotide sequence encoding said PGRN polypeptide, or variant thereof, is at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the nucleotide sequence of SEQ ID NO:8 or SEQ ID NO:15.
E12. The AAV vector of E1 to E5, wherein the nucleotide sequence encoding said PGRN polypeptide, or variant thereof, is identical to the nucleotide sequence of SEQ ID NO:8 or SEQ ID NO: 15.
E13. The AAV vector of E1 to E12, wherein said genome comprises at least one AAV inverted terminal repeat (ITR).
E14. The AAV vector of E13, wherein the nucleotide sequence of said ITR is wild-type.
E15. The AAV vector of E13, wherein the nucleotide sequence of said ITR is modified.
E16. The AAV vector of E15, wherein the nucleotide sequence of said ITR is modified to reduce or eliminate the ability of the ITR to undergo terminal resolution.
E17. The AAV vector of E15, wherein the nucleotide sequence of said ITR is modified to inactivate the terminal resolution site.
E18. The AAV vector of E15, wherein the nucleotide sequence of said ITR is modified to reduce or eliminate the ability of the ITR to support packaging into a capsid.
E19. The AAV vector of E15, wherein the nucleotide sequence of said ITR is modified to inactivate the D region.
E20. The AAV vector of E13 to E14, wherein said ITR is an AAV2 ITR.
E21. The AAV vector of E20, wherein said AAV2 ITR is truncated.
E22. The AAV vector of E13, wherein said ITR comprises, consists essentially of, or consists of the nucleotide sequence of SEQ ID NO:5, SEQ ID NO:12, SEQ ID NO:13, SEQ ID NO:20, or SEQ ID NO:21, or the complement or reverse complement of each of said sequences.
E23. The AAV vector of E13 to E14, wherein said ITR is other than an AAV2 ITR.
E24. The AAV vector of E1 to E23, wherein said vector comprises a first AAV ITR positioned at it 5′ terminus and a second AAV ITR positioned at its 3′ terminus.
E25. The AAV vector of E24, wherein said vector further comprises a third AAV ITR.
E26. The AAV vector of E25, wherein said third ITR is modified to inactivate the terminal resolution site.
E27. The AAV vector of E1 to E26, wherein said vector further comprises a transcription control region operably linked with said nucleotide sequence encoding said PGRN polypeptide, or variant thereof.
E28. The AAV vector of E27, wherein said transcription control region is constitutive.
E29. The AAV vector of E27, wherein said transcription control region is inducible.
E30. The AAV vector of E27, wherein said transcription control region is tissue or cell type specific.
E31. The AAV vector of E30, wherein said transcription control region is brain tissue specific, or neuron cell specific.
E32. The AAV vector of E30, wherein said transcription control region is more transcriptionally active in CNS neurons than in hepatocytes,
E33. The AAV vector of E27 to E32, wherein said transcription control region comprises a promoter sequence.
E34. The AAV vector of E33, wherein said transcription control region further comprises an enhancer sequence.
E35. The AAV vector of E34, wherein said enhancer sequence is positioned 5′ of the promoter.
E36. The AAV vector of E34, wherein said enhancer sequence is positioned 3′ of the promoter.
E37. The AAV vector of E33, wherein said promoter sequence is brain tissue specific, or neuron cell specific.
E38. The AAV vector of E34, wherein said enhancer sequence is brain tissue specific, or neuron cell specific.
E39. The AAV vector of E34, wherein each of said promoter sequence and enhancer sequence is brain tissue specific, or neuron cell specific.
E40. The AAV vector of E33, wherein said promoter and/or enhancer sequence is derived from a synapsin gene.
E41. The AAV vector of E34, wherein said promoter and/or enhancer sequence is derived from a synapsin gene selected from the group consisting of a mammalian synapsin gene, a mouse synapsin gene, a rat synapsin gene, a primate synapsin gene, a monkey synapsin gene, a chimpanzee synapsin gene, and a human synapsin 1 gene (SYN1).
E42. The AAV vector of E41, wherein said promoter sequence comprises, consists essentially of, or consists of the nucleotide sequence of SEQ ID NO:6, or a promoter functional subsequence, modification or variant thereof.
E43. The AAV vector of E41, wherein said enhancer sequence comprises, consists essentially of, or consists of the nucleotide sequence of SEQ ID NO:6, respectively, or an enhancer functional subsequence, modification or variant thereof.
E44. The AAV vector of E1 to E43, wherein said vector further comprises a 5′ untranslated region (UTR) sequence.
E45. The AAV vector of E44, wherein said 5′ UTR sequence is positioned 3′ of the promoter and/or enhancer sequence and 5′ of the nucleotide sequence encoding said PGRN polypeptide, or variant thereof.
E46. The vector of E44 to E45, wherein said 5′ UTR sequence is derived from a synapsin gene.
E47. The vector of E46, wherein said 5′ UTR sequence is derived from a synapsin gene selected from the group consisting of a mammalian synapsin gene, a mouse synapsin gene, a rat synapsin gene, a primate synapsin gene, a monkey synapsin gene, a chimpanzee synapsin gene, and a human synapsin 1 gene (SYN1).
E48. The AAV vector of E48, wherein said 5′ UTR sequence comprises, consists essentially of, or consists of the nucleotide sequence of SEQ ID NO:7, or a 5′ UTR functional subsequence, modification or variant thereof.
E49. The AAV vector of E1 to E48, wherein said vector further comprises a transcription termination signal sequence.
E50. The AAV vector of E49, wherein said transcription termination signal sequence is a polyadenylation (poly (A)) signal sequence.
E51. The AAV vector of E50, wherein said transcription termination signal sequence is derived from the bovine growth hormone (bGH) gene.
E52. The AAV vector of E51, wherein said transcription termination signal sequence comprises, consists essentially of, or consists of the nucleotide sequence of SEQ ID NO:9 or SEQ ID NO:10, or a transcription termination signal functional subsequence, modification or variant thereof.
E53. The AAV vector of E1 to E52, wherein said vector further comprises an intron sequence.
E54. The AAV vector of E53, wherein said intron sequence is positioned within and interrupts the nucleotide sequence encoding said PGRN polypeptide, or variant thereof.
E55. The AAV vector of E53, wherein said intron sequence does not interrupt the nucleotide sequence encoding said PGRN polypeptide, or variant thereof.
E56. The AAV vector of E55, wherein said intron sequence is positioned 5′ of the nucleotide sequence encoding said PGRN polypeptide, or variant thereof.
E57. The AAV vector of E56, wherein said intron sequence is positioned 3′ of the promoter and 5′ of the nucleotide sequence encoding said PGRN polypeptide, or variant thereof.
E58. The AAV vector of E1 to E57, wherein said vector further comprises a post-transcriptional regulatory element (PRE) sequence.
E59. The AAV vector of E58, wherein said PRE sequence is positioned 3′ of the nucleotide sequence encoding said PGRN polypeptide, or variant thereof, and 5′ of the transcription termination signal sequence.
E60. The AAV vector of E58 to E59, wherein said PRE sequence is a WPRE or a HPRE sequence.
E61. The AAV vector of E1 to E60, wherein said vector further comprises a binding site for a microRNA (miRNA).
E62. The AAV vector of E61, wherein said miRNA binding site is positioned 3′ of the nucleotide sequence encoding said PGRN polypeptide, or variant thereof, and 5′ of the transcription termination signal sequence.
E63. The AAV vector of E1 to E62, wherein said vector further comprises a stuffer or filler nucleotide sequence of sufficient length such that the entire length of said AAV vector inclusive of ITRs is approximately 3.5 to 5.0 kilobases.
E64. The AAV vector of E63, wherein said stuffer or filler nucleotide sequence is derived from a TATA binding protein (TBP) gene.
E65. The AAV vector of E64, wherein said stuffer or filler nucleotide sequence is a human TBP gene intron, or subsequence, modification or variant thereof.
E66. The AAV vector of E65, wherein said stuffer or filler nucleotide sequence comprises, consists essentially of, or consists of the nucleotide sequence of SEQ ID NO:11.
E67. The AAV vector of E1 to E66, wherein said vector comprises a first AAV ITR, a transcription control region in operable linkage with said nucleotide sequence encoding said PGRN polypeptide, or variant thereof, a transcription termination signal sequence, and a second AAV ITR.
E68. The AAV vector of E67, wherein said vector comprises in 5′ to 3′ order said first AAV ITR, said transcription control region in operable linkage with said nucleotide sequence encoding said PGRN polypeptide, or variant thereof, said transcription termination signal sequence, and said second AAV ITR.
E69. The AAV vector of E68, wherein said transcription control region comprises a promoter positioned 5′ of said nucleotide sequence encoding said PGRN polypeptide, or variant thereof and an enhancer positioned 5′ of said promoter.
E70. The AAV vector of E67 to E69, wherein said vector further comprises an intron positioned between said promoter and said nucleotide sequence encoding said PGRN polypeptide, or variant thereof.
E71. The AAV vector of E68 to E70, wherein said vector further comprises an intron positioned within and interrupting said nucleotide sequence encoding said PGRN polypeptide, or variant thereof.
E72. The AAV vector of E68 to E71, wherein said vector further comprises a PRE positioned between said nucleotide sequence encoding said PGRN polypeptide, or variant thereof and said poly (A) signal sequence.
E73. The AAV vector of E68 to E72, wherein said first AAV ITR is positioned at the 5′ terminus of said vector and said second AAV ITR is positioned at the 3′ terminus of said vector.
E74. The AAV vector of E73, wherein said vector further comprises a third AAV ITR positioned between said first and second AAV ITRs.
E75. The AAV vector of E74, wherein the terminal resolution site of said third AAV ITR is inactivated.
E76. The AAV vector of E68 to E75, wherein said transcription control region is brain tissue, or neuron cell specific.
E77. The AAV vector of E76, wherein said transcription control region comprises a promoter and/or enhancer sequence derived from a synapsin gene selected from the group consisting of a mammalian synapsin gene, a mouse synapsin gene, a rat synapsin gene, a primate synapsin gene, a monkey synapsin gene, a chimpanzee synapsin gene, and a human synapsin 1 gene (SYN1).
E78. The AAV vector of E77, wherein said transcription control region comprises, consists essentially of, or consists of the nucleotide sequence of SEQ ID NO:6, or a transcription control region functional subsequence, modification or variant thereof.
E79. The AAV vector of E67 to E78, wherein said transcription termination signal sequence is derived from the bovine growth hormone (bGH) gene.
E80. The AAV vector of E79, wherein said transcription termination signal sequence comprises, consists essentially of, or consists of the nucleotide sequence of SEQ ID NO:9 or SEQ ID NO:10, or a transcription termination signal functional subsequence, modification or variant thereof.
E81. The AAV vector of E67 to E80, further comprising a modified human TBP gene intron sequence positioned 3′ of said transcription termination signal sequence and 5′ of said second AAV ITR.
E82. The AAV vector of E1 to E5, wherein said vector comprises in 5′ to 3′ order:
   (a) a first AAV2 ITR,
   (b) a promoter sequence from a synapsin gene,
   (c) a 5′ UTR sequence from a synapsin gene,
   (d) a nucleotide sequence encoding a human PGRN polypeptide, or variant thereof, operably linked with said promoter sequence,
   (e) a transcription termination signal sequence from a bovine growth hormone (bGH) gene,
   (f) a sequence from a TBP gene intron, and
   (g) a second AAV2 ITR.
E83. The AAV vector of E82, wherein said promoter sequence from a synapsin gene comprises, consists essentially of, or consists of the nucleotide sequence of SEQ ID NO:6; said 5′ UTR sequence from a synapsin gene comprises, consists essentially of, or consists of the nucleotide sequence of SEQ ID NO:7; said nucleotide sequence encoding said PGRN polypeptide, or variant thereof, comprises, consists essentially of, or consists of the nucleotide sequence of SEQ ID NO:8 or SEQ ID NO:15; said transcription termination signal sequence from a bovine growth hormone (bGH) gene comprises, consists essentially of, or consists of the nucleotide sequence of SEQ ID NO:9 or SEQ ID NO:10; and said sequence from a TBP gene intron comprises, consists essentially of, or consists of the nucleotide sequence of SEQ ID NO:11.
E84. The AAV vector of E82 to E83, wherein each of said first and second AAV2 ITRs comprises, consists essentially of, or consists of the nucleotide sequence of SEQ ID NO:5, SEQ. ID NO: 12, SEQ ID NO:13, SEQ ID NO:20, or SEQ ID NO:21, or the complement or reverse complement of each of said sequences.
E85. The AAV vector of E82 to E84, wherein the nucleotide sequence of said vector comprises, consists essentially of, or consists of the nucleotide sequence of SEQ ID NO:19, or the reverse complement thereof.
E86. The AAV vector of E1 to E85, wherein said vector is equal to or less than 5 kilobases in length.
E87. The AAV vector of E1 to E86, wherein said vector is equal to or less than 4 kilobases in length.
E88. An AAV vector comprising an AAV capsid and the AAV vector of E1 to E87, wherein said vector is encapsidated by said capsid.
E89. The AAV vector of E88, wherein said AAV capsid is at least partially neuronotropic.
E90. The AAV vector of E89, wherein said AAV capsid is capable of crossing the blood brain barrier (BBB) in non-human primates, or in humans.
E91. The AAV vector of E90, wherein said AAV capsid is at least as, or more efficient crossing the BBB as compared to AAV9 capsid.
E92. The AAV vector of E89 to E90, wherein said AAV capsid is selected from the group of consisting of: AAV1, AAV2, AAV4, AAV5, AAV7, AAV8, AAV9, AAV-rh. 10, AAVv66, AAV-PHP.B, AAV-PHP.B/eB, AAV PHP.eB, AAV PHP.S, AAV-DJ, MNM008, MNM004, 9P31, 9P801, AAV-F, AAV-S, CAP-B10, CAP-B22, PHP.V1, AAV9-retro, T2 3Y+T+dH, AAV8 THR, AAV2.5, AAV-B1, AAV-AS, AAV-BR1, AAV SCH9, AAV4.18, AAV2-retro, AAV2 HBKO, AAV-TT, and AAV-801.
E93. The AAV vector of E89 to E92, wherein said AAV capsid is an AAV-801 capsid and comprises a VP3 protein consisting of the amino acid sequence of SEQ ID NO:3.
E94. The AAV vector of E93, wherein said AAV capsid further comprises a VP1 protein consisting of the amino acid sequence of SEQ ID NO:1, or a VP2 protein consisting of the amino acid sequence of SEQ ID NO:2.
E95. The AAV vector of E88 to E94, wherein said vector is a single-stranded DNA genome.
E96. The AAV vector of E88 to E94, wherein said vector is a self-complementary DNA genome.
E97. The AAV vector of E95 to E96, wherein said vector is in the plus polarity.
E98. The AAV vector of E95 to E96, wherein said vector is in the minus polarity.
E99. An AAV vector comprising an AAV capsid encapsidating an AAV vector, wherein said AAV capsid is an AAV-801 capsid, and wherein the nucleotide sequence of said vector comprises or consists of the nucleotide sequence of SEQ ID NO:17, or the reverse complement thereof.
E100. A pharmaceutical composition comprising the AAV vector of E88 to E100 and a pharmaceutically acceptable excipient.
E101. A method of preventing or treating a disease or disorder in a human subject caused by a deficiency of human PGRN polypeptide comprising administering to said subject an amount of the AAV vector or composition of E1 to E100 effective to increase the amount of PGRN polypeptide, or variant thereof, in at least one biofluid, tissue or cell of said subject.
E102. The method of E101, wherein said method is effective to increase the amount of PGRN polypeptide, or variant thereof, in cerebrospinal fluid (CSF) of said subject to at least or about 5%, 10%, 15%, 20%, 25%, 30%, 35%, 40%, 45%, 50%, 60%, 70%, 80%, 90%, or 100% of the amount of endogenous PGRN polypeptide in the CSF of healthy humans, for example, 6 ng/ml.
E103. The method of E101, wherein said method is effective to increase the amount of PGRN polypeptide, or variant thereof, in the brain of said subject to at least or about 5%, 10%, 15%, 20%, 25%, 30%, 35%, 40%, 45%, 50%, 60%, 70%, 80%, 90%, or 100% of the amount of PGRN polypeptide in brains of healthy humans.
E104. The method of E101, wherein said method is effective to increase the amount of PGRN polypeptide, or variant thereof, in the spinal cord of said subject to at least or about 5%, 10%, 15%, 20%, 25%, 30%, 35%, 40%, 45%, 50%, 60%, 70%, 80%, 90%, or 100% of the amount of PGRN polypeptide in spinal cords of healthy humans.
E105. The method of E101, wherein said method is effective to increase the amount of PGRN polypeptide, or variant thereof, in serum of said subject to at least or about 5%, 10%, 15%, 20%, 25%, 30%, 35%, 40%, 45%, 50%, 60%, 70%, 80%, 90%, or 100% of the amount of PGRN polypeptide in the serum of healthy humans.
E106. The method of E101, wherein said method is effective to increase the amount of bis(monoacylglycero)phosphate (BMP) in the brain of said subject to at least or about 5%, 10%, 15%, 20%, 25%, 30%, 35%, 40%, 45%, 50%, 60%, 70%, 80%, 90%, or 100% of the amount of BMP in brain of healthy humans, wherein said BMP can be any species of BMP, such as BMP 18:1/18:1 or BMP 22:6/22:6.
E107. The method of E101, wherein said method is effective to reduce β-hexosaminidase (HexA) enzymatic activity in the brain of said subject to at most or less than about 95%, 90%, 85%, 80%, 75%, 70%, 65%, 60%, 55%, 50%, 45%, 40%, 35%, 30%, 25%, 20%, 15%, 10%, or 5%, of the HexA enzymatic activity prior to treatment.
E108. The method of E101, wherein said method is effective to reduce β-galactosidase (β-Gal) enzymatic activity in the brain of said subject to at most or less than about 95%, 90%, 85%, 80%, 75%, 70%, 65%, 60%, 55%, 50%, 45%, 40%, 35%, 30%, 25%, 20%, 15%, 10%, or 5%, of the β-Gal enzymatic activity prior to treatment.
E109. The method of E101, wherein said method is effective to reduce TDP43 fragmentation in the brain of said subject to at most or less than about 95%, 90%, 85%, 80%, 75%, 70%, 65%, 60%, 55%, 50%, 45%, 40%, 35%, 30%, 25%, 20%, 15%, 10%, or 5%, of TDP43 fragmentation prior to treatment.
E110. The method of E101, wherein said method is effective to reduce lipofucin levels in the brain of said subject to at most or less than about 95%, 90%, 85%, 80%, 75%, 70%, 65%, 60%, 55%, 50%, 45%, 40%, 35%, 30%, 25%, 20%, 15%, 10%, or 5%, of the lipofucin levels prior to treatment.
E111. A method of reducing the frequency or severity of at least one symptom or sign in a human subject caused by a deficiency of human PGRN polypeptide comprising administering to said subject an amount of the AAV vector or composition of E1 to E100 effective to reduce the frequency or severity of such symptom or sign.
E112. The method of E111, wherein the symptom or sign is characteristic of frontotemporal lobar degeneration (FTLD) including, for example, FTLD-TDP type A.
E113. The method of E112, wherein the symptom or sign is atrophy in a brain region selected from the group consisting of: frontal lobe, anterior temporal lobe, medial temporal lobe, posterior temporal lobe, orbitofrontal cortex, anterior cingulate gyrus, inferior parietal lobe, striatum, and thalamus.
E114. The method of E112, wherein the symptom or sign is a behavioral change characteristic of behavioral-variant frontotemporal dementia (BV-FTD).
E115. The method of E112, wherein the symptom or sign is a behavioral change characteristic of non-fluent variant primary progressive aphasia (NFV-PPA).
E116. The method of E114 or E115, wherein the behavioral change is selected from the group consisting of: impaired word finding, apraxia of speech, agrammatism, impaired confrontation naming, impaired single-word comprehension, phonological errors, word repetition errors, sentence repetition errors, sentence comprehension errors, surface dyslexia, delusions, and hallucinations.
E117. The method of E112, wherein the symptom or sign is characteristic of Parkinsonism or corticobasal syndrome (CBS).
E118. The method of E101 to E117, wherein said subject is diagnosed with frontotemporal lobar degeneration (FTLD) or frontotemporal dementia (FTD).
E119. The method of E101 to E118, wherein the deficiency of PGRN polypeptide in said subject is caused by a homozygous or heterozygous mutation in the GRN gene encoding PGRN polypeptide that reduces the amount or activity of PGRN polypeptide relative to healthy humans.
E120. A method of preventing or treating frontotemporal dementia in a human subject comprising administering to said subject a prophylactically or therapeutically effective amount of an AAV vector or composition of E1 to E100 effective to prevent or treat frontotemporal dementia in said subject.
E121. The method of E101 to E120, wherein the effective amount of said AAV vector is a dose ranging from 1×10¹⁰ to 1×10¹⁵ vectors per kilogram (vg/kg) of subject body weight.
E122. The method of E101 to E121, wherein said AAV vector or composition is administered to said subject intracerebroventricularly.
E123. The method of E101 to E121, wherein said AAV vector or composition is administered to said subject intrathecally.
E124. The method of E101 to E121, wherein said AAV vector or composition is administered to said subject intravenously.
E125. Use of the AAV vector of E1 to E100 in the manufacture of a medicament for treating or preventing frontotemporal dementia in a human subject.
E126. A DNA plasmid comprising the nucleotide sequence of the AAV vector of E1 to E87.
E127. A host cell for AAV vector production comprising the DNA plasmid of E126.
E128. The host cell of E127, wherein said host cell is a HEK293 cell.
E129. The host cell of E127 to E128, wherein said host cell further comprises a gene encoding an AAV Rep protein, such as contained in a DNA plasmid.
E130. The host cell of E127 to E129, wherein said host cell further comprises a gene encoding an AAV VP1 capsid protein, such as contained in a DNA plasmid.
E131. The host cell of E127 to E130, wherein said host cell further comprises a gene coding for a viral helper factor, such as contained in a DNA plasmid.
E132. A method of making a AAV vector, comprising: incubating the host cell of E131 under conditions sufficient to allow the production of AAV vectors, and purifying the AAV vectors produced thereby.
E133. An AAV vector produced by the method of E132.

### BRIEF DESCRIPTION OF THE DRAWINGS

FIG. 1. Biacore sensorgrams of human His-tagged full-length PGRN and PGRNΔ3 proteins binding to immobilized human sortilin receptor (SORT1) and human prosaposin (PSAP). Panel A shows binding of full-length PGRN to SORT1. Panel B shows full-length PGRN and PGRNΔ3 binding to PSAP (top and bottom, respectively), whereas panel C shows similar binding to PSAP from an alternative source.

FIG. 2A. Levels of mRNA encoding human progranulin in motor neurons differentiated from human iPSC cells transduced with AAVDJ vectors for expressing human full-length and Δ3 truncated PGRN protein.

FIG. 2B. Progranulin protein levels measured in conditioned media of motor neurons differentiated from human iPSC cells transduced with AAVDJ vectors for expressing human full-length and 43 truncated PGRN protein.

FIG. 3A. Levels of mRNA encoding human progranulin in cortical glutaminergic neurons differentiated from human iPSC cells transduced with AAV6 vectors for expressing human full-length and Δ3 truncated PGRN protein.

FIG. 3B. Progranulin protein levels measured in conditioned media of cortical glutaminergic neurons differentiated from human iPSC cells transduced with AAV6 vectors for expressing human full-length and Δ3 truncated PGRN protein. Also shown are results from a related experiment in which glutaminergic neurons were differentiated from iPSC cells from a human FTD patient and then transduced with AAV6 vectors for expressing human 43 truncated PGRN protein.

FIG. 4. Human PGRNΔ3 protein concentration released into media by glutamatergic neurons derived from iPS cells after transduction by AAV801-PGRNΔ3 vector, compared to untreated control cells, and neurons transduced with the control vector AAV801-Luc. Statistical analysis employed one-way ANOVA test followed by post-hoc analysis using Dunnet's multiple comparisons test. P value ****: <0.0001.

FIG. 5. β-hexosaminidase enzymatic activity in lysates of glutamatergic neurons differentiated PSC cells. Left, results for control neurons from healthy iPSC cells that were untreated, and transduced with a control vector, AAV801-Luc, and vector expressing a GRN transgene, AAV801-PGRNA vector. Right, results for neurons from FTD iPSC cells that were untreated and transduced with AAV801-Luc and AAV801-PGRNA vector. Statistical analysis employed a two-way ANOVA test followed by post-hoc analysis using Šíd\dot{a}k's multiple comparisons test. P values ***: =<0.0001; ***: =0.0004; ns: non-significant.

FIG. 6A. Transduction efficiency in brain tissue of AAV9 vectors for expressing human full-length and 43 truncated PGRN administered ICV to neonatal mice.

FIG. 6B. Progranulin protein levels measured in CSF from neonatal mice 4 weeks after administration of AAV9 vectors for expressing human full-length and 43 truncated PGRN.

FIG. 6C. Progranulin protein levels measured in CSF from neonatal mice 4 weeks after administration of AAV9 vectors for expressing human full-length and Δ3 truncated PGRN. Data normalized to amount of endogenous mouse PGRN in samples.

FIG. 6D. Progranulin protein levels measured in serum from neonatal mice 4 weeks after administration of AAV9 vectors for expressing human full-length and 43 truncated PGRN.

FIG. 7A. Progranulin protein detected by immunohistochemistry in brain section from neonatal mouse 4 weeks after administration of negative control AAV9 vector for expressing green fluorescent protein.

FIG. 7B. Progranulin protein detected by immunohistochemistry in brain section from neonatal mouse 4 weeks after administration of AAV9 vector for expressing human full-length PGRN.

FIG. 7C. Progranulin protein detected by immunohistochemistry in brain section from neonatal mouse 4 weeks after administration of AAV9 vector for expressing human 43 truncated PGRN.

FIG. 8A. Transduction efficiency in brain tissue of AAV1, AAVDJ, and AAV9 vectors for expressing human full-length and 43 truncated PGRN administered ICV to mice at 6 months of age.

FIG. 8B. Progranulin protein levels measured in CSF from mice 3 months after administration of AAV1, AAVDJ, and AAV9 vectors for expressing human full-length and 43 truncated PGRN, when the mice were 6 months of age.

FIG. 8C. Progranulin protein levels measured in CSF from mice 6 months after administration of AAV9 vectors for expressing human full-length and Δ3 truncated PGRN, when the mice were 6 months of age.

FIG. 9A. Transduction efficiency in brain tissue of AAVPHP.B vectors for expressing human full-length and 43 truncated PGRN administered intravenously to mice at 6 weeks of age.

FIG. 9B. Progranulin protein levels measured in brain tissue from mice 4 weeks after administration of AAVPHP.B vectors for expressing human full-length and 43 truncated PGRN, when the mice were 6 weeks of age.

FIG. 9C. Progranulin protein levels measured in CSF from mice 4 weeks after administration of AAVPHP.B vectors for expressing human full-length and 43 truncated PGRN, when the mice were 6 weeks of age.

FIG. 10. Micrographs of hippocampal region, including CA3, from mouse 3 months after administration of negative control AAV9 vector for expressing green fluorescent protein, when test animal was 6 months of age. Upper left micrograph shows negative staining for human progranulin protein. Lower left micrograph shows negative staining for endogenous mouse Iba-1, a marker for microglial activation. Right micrograph shows intact tissue cellular architecture by H&E staining.

FIG. 11. Micrographs of hippocampal region, including CA3, from mouse 3 months after administration of AAV9 vector for human full-length PGRN, when test animal was 6 months of age. Upper left micrograph shows positive and focal staining for human progranulin protein. Lower left micrograph shows positive staining for endogenous mouse Iba-1. Right micrograph shows decreased cellularity by H&E staining.

FIG. 12. Micrographs of hippocampal region, including CA3, from mouse 3 months after administration of AAV9 vector for human Δ3 truncated PGRN, when test animal was 6 months of age. Upper left micrograph shows positive and diffuse staining for human progranulin protein. Lower left micrograph shows no or minimal staining for endogenous mouse Iba-1. Right micrograph shows normal cellularity by H&E staining.

FIG. 13A. Vector copy (VGC) numbers in liver samples harvested from Grn^{-/-} KO mice administered 1e11 vg AAV9-PGRNΔ3 vectors into one hemisphere via the intracerebroventricular (ICV) route of delivery. Age matched wild-type mice and Grn^{-/-} KO mice administered PBS by the same route served as controls. VGC numbers are normalized to amount of cellular genomic DNA (μg gDNA).

FIG. 13B. Vector copy (VGC) numbers in brain samples harvested from Grn^{-/-} KO mice administered 1e11 vg AAV9-PGRNΔ3 vectors into one hemisphere via the ICV route of delivery. Age matched wild-type mice and Grn^{-/-} KO mice administered PBS by the same route served as controls. VGC numbers are normalized to amount of cellular genomic DNA (μg gDNA). Pink dots represent results from samples of brain harvested from the injected hemisphere, and black dots represent data from the contralateral hemisphere.

FIG. 13C. Quantity of hGRNA3 mRNA in brain samples harvested from Grn^{-/-} KO mice administered 1e11 vg AAV9-PGRNΔ3 vectors into one hemisphere via the ICV route of delivery. Age matched wild-type mice and Grn^{-/-} KO mice administered PBS by the same route served as controls. RNA levels are normalized to amount of RNA expressed from a housekeeping gene. Pink dots represent results from samples of brain harvested from the injected hemisphere, and black dots represent data from the contralateral hemisphere.

FIGS. 13D-13F. Concentration of PGRNΔ3 protein in fluid and tissue samples from Grn^{-/-} KO mice administered 1e11 vg AAV9-PGRNΔ3 vectors into one hemisphere via the ICV route of delivery. Age matched wild-type mice and Grn^{-/-} KO mice administered PBS by the same route served as controls. Pink dots represent results from fluid and tissue samples taken from the injected hemisphere, and black dots represent data from the contralateral hemisphere. FIG. 13D shows results for cerebrospinal fluid (CSF) samples. FIG. 13E shows results for serum samples. FIG. 13F shows results for brain tissue samples.

FIGS. 14A-14B. Concentration of BMP 18:1/18:1 species (FIG. 14A) and BMP 22:6/22:6 species (FIG. 14B) in brain samples harvested from Grn^{-/-} KO mice administered 1e11 vg AAV9-PGRNΔ3 vectors into one hemisphere via the ICV route of delivery. Age matched wild-type mice and Grn^{-/-} KO mice administered PBS by the same route served as controls. Pink dots represent results from samples of brain harvested from the injected hemisphere, and black dots represent data from the contralateral hemisphere. Statistical analysis employed a one-way ANOVA followed by post-hoc analysis with Dunnett's multiple comparisons test. P-values ****: <0.0001; **: <0.01, *: <0.05; ns: non-significant.

FIGS. 15A-15B. Enzymatic activity of β-hexosaminidase (FIG. 15A) and β-galactosidase (FIG. 15B) in brain samples harvested from Grn^{-/-} KO mice administered 1e11 vg AAV9-PGRNΔ3 vectors into one hemisphere via the ICV route of delivery. Age matched wild-type mice and Grn^{-/-} KO mice administered PBS by the same route served as controls. Pink dots represent results from samples of brain harvested from the injected hemisphere, and black dots represent data from the contralateral hemisphere. Statistical analysis employed a one-way ANOVA followed by post-hoc analysis with Dunnett's multiple comparisons test. P-values ****: <0.0001; **: <0.01; ns: non-significant.

FIGS. 16. TDP43 fragmentation in brain samples from Grn^{-/-} KO mice administered 1e11 vg AAV9-PGRNΔ3 vectors into one hemisphere via the ICV route of delivery, as well as age matched control wild-type mice and Grn^{-/-} KO mice administered PBS. Semi-quantitative Western blot analysis was used to estimate fragmentation as the ratio of the staining intensity of full-length TDP43 protein to its 20 kDa fragment, with lower ratios indicating more fragmentation. Statistical analysis employed one way ANOVA followed by post-hoc analysis with Dunnett's multiple comparisons test. P-value *: <0.05; ns: non-significant.

FIGS. 17A-17E. Quantification of lipofuscin accumulation in different regions of brain samples harvested from Grn^{-/-} KO mice administered 1e11 vg AAV9-PGRNΔ3 vectors into the right hemisphere via the ICV route of delivery. Age matched wild-type mice administered the control vector AAV9-Luc and Grn^{-/-} KO mice administered PBS by the same routes served as controls. FIG. 17A shows results for the CA2/3 hippocampal area. FIG. 17B shows results for the entire hippocampus. FIG. 17C shows results for the prefrontal cortex. FIG. 17D shows results for the thalamus. FIG. 17E shows results for the entire brain. Legend indicating “right” refers to data collected from the same hemisphere of the brain into which GRN vector was injected, whereas “left” refers to the contralateral hemisphere.

FIG. 18A. Vector copy (VGC) numbers in liver samples harvested from Grn null knock-in (KI) mice administered AAVPHP.B-PGRNΔ3 vectors (5e12 vg/kg or 1e13 vg/kg) via the intravenous (IV) route of delivery. Age matched Grn null KI mice administered PBS by the same route served as controls. VGC numbers are normalized to amount of cellular genomic DNA (μg gDNA). Statistical analysis employed one way ANOVA followed by post-hoc analysis with Dunnett's multiple comparisons test. P-values ****: <0.0001; *: <0.05; ns: non-significant.

FIG. 18B. Vector copy (VGC) numbers in brain samples harvested from Grn null knock-in (KI) mice administered AAVPHP.B-PGRNΔ3 vectors (5e12 vg/kg or 1e13 vg/kg) via the intravenous (IV) route of delivery. Age matched Grn null KI mice administered PBS by the same route served as controls. VGC numbers are normalized to amount of cellular genomic DNA (μg gDNA). Statistical analysis employed one way ANOVA followed by post-hoc analysis with Dunnett's multiple comparisons test. P-values ****: <0.0001; *: <0.05; ns: non-significant.

FIG. 18C. Quantity of hGRNA3 mRNA in brain samples harvested from Grn null KI mice administered AAVPHP.B-PGRNΔ3 vectors (5e12 vg/kg or 1e13 vg/kg) via the intravenous (IV) route of delivery. Age matched wild-type mice and Grn null KI mice administered PBS by the same route served as controls. RNA levels are normalized to amount of RNA expressed from a housekeeping gene. Statistical analysis employed one way ANOVA followed by post-hoc analysis with Dunnett's multiple comparisons test. P-values ****: <0.0001; *: <0.05; ns: non-significant,

FIGS. 18D-18F. Concentration of PGRNΔ3 protein in fluid and tissue samples from Grn null KI mice administered AAVPHP.B-PGRNΔ3 vectors (5e12 vg/kg or 1e13 vg/kg) via the intravenous (IV) route of delivery. Age matched wild-type mice and Grn null KI mice administered PBS by the same route served as controls. FIG. 18D shows results for cerebrospinal fluid (CSF) samples. FIG. 18E shows results for serum samples. FIG. 18F shows results for brain tissue samples. Statistical analysis same as described for FIG. 18C.

FIGS. 18G-18H. Concentration of BMP 18:1/18:1 species (FIG. 18G) and BMP 22:6/22:6 species (FIG. 15H) in brain samples harvested from Grn null KI mice administered AAVPHP.B-PGRNΔ3 vectors (5e12 vg/kg or 1e13 vg/kg) via the intravenous (IV) route of delivery. Age matched wild-type mice and Grn null KI mice administered PBS by the same route served as controls. Statistical analysis same as described for FIG. 18C.

FIG. 181. Enzymatic activity of β-hexosaminidase in brain samples harvested from Grn null KI mice administered AAVPHP.B-PGRNΔ3 vectors (5e12 vg/kg or 1e13 vg/kg) via the intravenous (IV) route of delivery. Age matched wild-type mice and Grn null KI mice administered PBS by the same route served as controls. Statistical analysis same as described for FIG. 18C.

FIG. 19A. Concentration of PGRNΔ3 protein in CSF samples from cynomolgus monkeys 14 and 30 days after being administered AAV801-PGRNΔ3 vectors (5e12 vg/kg or 2e13 vg/kg) or AAV9-PGRNΔ3 vectors (2e13 vg/kg) via the intravenous (IV) route of delivery. Two animals received each vector and dose. PGRNΔ3 protein was undetectable in one test animal that received the low dose of AAV801-PGRNΔ3, and in both test animals that received 2e13 vg/kg AAV9-PGRNΔ3. Dotted line labeled “target” indicates progranulin levels naturally occurring in humans.

FIG. 19B. Concentration of PGRNΔ3 protein in serum samples from cynomolgus monkeys before IV administration of AAV801-PGRNΔ3 vectors (5e12 vg/kg or 2e13 vg/kg) or AAV9-PGRNΔ3 vectors (2e13 vg/kg), and on days 3, 7, 14, 21, and 28 after treatment. Two animals received each vector and dose.

FIG. 20. Vector copy (VGC) numbers in brain and other tissue samples harvested from cynomolgus monkeys 28 days after IV administration of AAV801-PGRNΔ3 vectors (5e12 vg/kg or 2e13 vg/kg) or AAV9-PGRNΔ3 vectors (2e13 vg/kg). Multiple brain regions, spinal cord, and peripheral neural and non-neural tissues were sampled. VGC numbers are normalized to amount of cellular genomic DNA (μg gDNA).

FIG. 21. Quantity of hGRNA3 mRNA in brain and other tissue samples harvested from cynomolgus monkeys 28 days after IV administration of AAV801-PGRNΔ3 vectors (5e12 vg/kg or 2e13 vg/kg) or AAV9-PGRNΔ3 vectors (2e13 vg/kg). Multiple brain regions, spinal cord, and peripheral neural and non-neural tissues were sampled. RNA levels are normalized to amount of RNA expressed from a housekeeping gene.

FIGS. 22A-22G. Representative images of PGRNΔ3 transgene expression visualized by in situ hybridization performed on brain and spinal cord tissue samples from a male cynomolgus monkey 28 days after IV administration of 2e13 vg/kg AAV801-PGRNΔ3 vectors, FIG. 22A shows results for motor cortex. FIG. 22B shows results for entorhinal cortex. FIG. 22C shows results for hippocampal pyramidal cells. FIG. 22D shows results for thalamus. FIG. 22E shows results for the dentate nucleus. FIG. 22F shows results for the spinal cord. FIG. 22G shows results for thalamus from a negative control animal. In the images, mRNA is labelled in red and nuclei in blue.

FIG. 23. Concentration of PGRNΔ3 protein in brain and other tissue samples harvested from cynomolgus monkeys 28 days after IV administration of AAV801-PGRNΔ3 vectors (5e12 vg/kg or 2e13 vg/kg) or AAV9-PGRNΔ3 vectors (2e13 vg/kg) compared to levels of endogenous macaque progranulin. Multiple brain regions, spinal cord, and peripheral neural and non-neural tissues were sampled.

### DETAILED DESCRIPTION

The following discussion is directed to various embodiments. The disclosure is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to intimate that the scope of the disclosure, including the claims, is limited to that embodiment.

### Certain Definitions

As used herein, “adeno-associated virus vector” means an adeno-associated virus (AAV) comprising a naturally occurring or non-naturally occurring AAV capsid encapsidating a vector. Adeno-associated virus vector may be abbreviated “AAV vector,” and depending on context, may be referred to by synonymous terms, such as “recombinant AAV vector,” “rAAV vector,” “rAAV,” or just “vector.”

As used herein, “vector” means an AAV genome modified both to include a heterologous nucleotide sequence and to render any AAV vector containing the vector replication incompetent, such as by inactivating or deleting an endogenous AAV rep and/or cap gene.

As used herein, “heterologous nucleotide sequence” means a nucleotide sequence that is introduced into an organism (including a virus) from a different organism (including an organism). The sequence of a heterologous nucleotide sequence may be the same as one that occurs in nature, or may be a modified version thereof, or even partially or entirely synthetic.

As used herein, “expression cassette” means a nucleotide sequence comprising a transgene operably linked with regulatory regions or elements for controlling the initiation and termination of transcription of the transgene from DNA into RNA.

As used herein, “transgene” means a nucleotide sequence that encodes at least one polypeptide, and/or the nucleotide sequence coding for at least one functional RNA molecule. Transgene may be referred to by the synonymous term “gene of interest.”

As used herein, “host cell” means a cell in which AAV vectors are produced. Producer cells and packaging cells are examples of host cells. Host cells can be mammalian or insect, or from other organisms, whether single or multi-cellular.

As used herein, the term “purify,” and the related terms “purified” and “purification,” when used in connection with an AAV vector, or sample or preparation thereof, indicates a relative increase or improvement in purity compared with a starting material containing the vector, and/or a prior intermediate purification step in some scheme of sequential purification steps intended to purify the biological product, and does not require a particular qualitative or quantitative degree of purity, unless otherwise specified.

As used herein, “transduction” means the introduction into a target cell of the genome of an AAV vector. Transduction is distinguished from infection, the latter term being used to refer to the introduction into a cell of the genome of a replication competent adeno-associated virus.

As used herein, “target cell” means a cell that an AAV vector is designed or intended to transduce, or is experimentally observed to be transduced by an AAV vector, whether in vitro, or in vivo in a subject.

As used herein, “subject” means an organism to which an AAV vector is administered for purposes of preventing or treating a disease, disorder, or condition.

### Frontotemporal Dementia and Frontotemporal Lobar Degeneration

Frontotemporal dementia (FTD) refers to a clinical syndrome characterized by progressively worsening deficits in language, behavior and executive function associated with the selective neurodegeneration of the frontal and temporal cortical lobes (frontotemporal lobar degeneration, or FTLD), as opposed to the more global neurodegeneration commonly seen in Alzheimer's disease and certain other dementias. The age of onset for many patients with FTD occurs in their forties to early sixties, with a prevalence of 10% for patients younger than 45, 60% between the ages of 45 to 64, and 30% for those older than 64. After onset, symptoms worsen progressively, and depending on the subtype of FTD, the survival time is 6 to 11 years, averaging 8, although some aggressive subtypes can lead to death in as few as 2 years.

FTD presents with variable neurological symptoms, depending on the underlying pattern of neurodegeneration, and three clinical variants have been defined. Behavioral-variant frontotemporal dementia (BV-FTD) is associated with early behavioral and executive deficits, non-fluent variant primary progressive aphasia (NFV-PPA) is typified by progressive deficits in speech, grammar, and word output, and semantic-variant primary progressive aphasia (SV-PPA) is characterized by a progressive disorder of semantic knowledge and naming. Diagnosis of an FTD patient with one of the clinical variants depends on the prevailing behavioral and language deficits, particularly early in the disease process. Diagnosis with the BV-FTD variant requires at least three of the following behavioral changes: disinhibition; apathy or inertia; loss of sympathy or empathy; stereotypical, compulsive, or perseverative behavior; hyperorality or dietary changes; and executive deficits with relative sparing of visuospatial skills and memory. The primary progressive aphasia (PPA) variants both require presence of language deficits, of which aphasia is the most prominent initially. Significant early deficits in episodic memory, visual memory, or visuoperceptual skills, or behavioral disturbances rule out PPA. Then, PPA would be further distinguished into the semantic versus non-fluent variants. For SV-PPA, the patient will present with impaired confrontation naming and single-word comprehension, and at least three of the following functional deficits or capabilities relating to language: impaired object knowledge; surface dyslexia or dysgraphia; spared repetition; and spared speech production. NFV-PPA, on the other hand, requires at least one of agrammatism in production of speech, or apraxia of speech, and at least two of impaired comprehension of complex sentences, spared single-word comprehension, and spared object knowledge. Motor symptoms can also affect a minority of FTD patients. A little over 12% of BV-FTD patients will also develop motor neuron disease, and less often in FTD patients with the PPA variants. About 20% of FTD patients also present with parkinsonism symptoms and may also experience features of corticobasal syndrome or progressive supranuclear palsy syndrome.

Although many of FTD's symptoms are also experienced by patients with other types of dementias or psychiatric diseases, the brains of patients with FTD will exhibit characteristic atrophy of the frontal or temporal lobes, with atrophy of the frotoinsular region being particularly indicative of FTD. The patterns of cortical atrophy associated with FTD can be detected using structural neuroimaging methods, such as MRI or CT, or functional methods, such as fluorodeoxyglucose PET, functional MRI, and SPECT.

Confirming FTD usually requires examining the brain of suspected cases to detect the neuropathological changes associated with the frontotemporal lobar degeneration (FTLD) disease process. While FTLD is characterized generally by neuronal loss, gliosis, and microvacuolar changes in the frontal lobes, anterior temporal lobes, anterior cingulate cortex, and insular cortex, nearly all cases of can be differentiated into three major types FTLD-tau, FTLD-TDP, and FTLD-FUS, based on detecting the corresponding presence of abnormal deposits of certain proteins, which are also correlated with certain characteristic patterns of neurogeneration. FTLD-tau, which accounts for about 36-50% of all FTLD cases, is defined by the presence on neuropathological examination of deposits of microtubule-associated protein tau (MAPT) and is associated with the FTLD subtypes of Pick's disease, corticobasal degeneration, and progressive supranuclear palsy. FTLD-FUS, accounting for about 10% of FTLD cases, is defined by the presence of brain deposits of fused-in-sarcoma (FUS) protein and is associated with early onset of FTD behavioral symptoms and absence of motor and language deficits. FTLD-TDP is most prevalent, accounting for about 50% of FTLD cases, and is defined by brain deposits of the TAR DNA-binding protein with molecular weight 43 kDa (TDP-43).

FTLD-TDP is further distinguished into three subtypes A, B, and C, based both on patterns of abnormal protein deposition in the brain, and characteristic patterns of neurodegeneration. FTLD-TDP type A is associated with asymmetrical dorsal atrophy including frontal and temporal lobes (anterior, medial, and posterior regions), orbitofrontal cortex, anterior cingulate gyrus, inferior parietal lobe, striatum, and thalamus. FTLD-TDP type B is associated with atrophy involving the medial and polar temporal lobe, anterior insular, cingulate and medial prefrontal cortices, and orbitofrontal cortex, with the frontal lobe being more severely affected in the posterior areas. FTLD-TDP type C is associated with right or left-predominant anterior temporal lobe atrophy, additionally involving the amygdala, hippocampus, orbitofrontal cortex, and insular cortex. Different FTLD-TDP subtypes reportedly also correlate with certain FTD symptoms. Thus, TDP type A accounts for about 50% of NF-PPA cases, 25% of suspected corticobasal degeneration cases, but only a small proportion of BV-FTD (with or without motor neuron disease); type B accounts for about two-thirds of FTD cases presenting with motor neuron disease and 25% of BV-FTD overall; and type C accounts for about 90% of all cases of SV-PPA, or temporal-variant BV-FTD. Bang, J. et al., Lancet, 386:1672-82, 2015.

Human genetics studies have established that perhaps as many as 40% of FTLD cases are familial, and identified mutations in three genes—90RF72, MAPT, and GRN—which are responsible for about 60% of inherited cases overall. The GRN gene encodes progranulin (PGRN), a secreted protein implicated in functions as diverse as cell-cycle regulation, wound repair, axon growth, and inflammation. PGRN binds tumor necrosis factor receptor (TNFR), among other receptors, suggesting a mechanism for modulating inflammation caused by TNFα or other inflammatory mediators. Heterozygous loss-of-function mutations in the GRN gene result in haploinsufficiency with substantial loss of PGRN concentrations in cerebrospinal fluid (CSF) and serum. Over 70 pathogenic mutations have been found in GRN gene leading to FTD, with the majority being non-sense mutations triggering the degradation of the mRNA encoding progranulin. Present in about 5-20% of familial FTD cases, GRN mutations leading to haploinsufficiency and reduced PGRN production are associated with characteristic patterns of FTD clinical syndromes, brain atrophy, and neuropathology. As reported by Bang et al., GRN loss of function mutations are associated with neurological symptoms and signs of BV-FTD, NFV-PPA, parkinsonism, and corticobasal syndrome (CBS) and patterns of neurodegeneration that are asymmetrical, predominantly affecting the anterior temporal lobe, the temporo-parietal lobe, the frontal lobe (left side more associated with PPA syndromes, and right side more associated with BV-FTD symptomology), anterior cingulate cortex, and insular cortex. Additionally, on neuropathological examination, the brains of FTD patients with GRN mutations often present with cellular changes and TDP protein deposits that characteristic of FTLD-TDP type A.

### Adeno-Associated Virus (AAV)

The disclosure provides vectors created from recombinantly modified adeno-associated virus (AAV). AAV vectors are capable of delivering genes, which may be under the control of transcriptional and other regulatory elements, into targeted cells via transduction. By supplying a functional copy of a gene to a target cell in which the endogenous version is missing or mutated, AAV vectors are useful in gene therapy for a variety of diseases and disorders.

AAV is a small non-enveloped, apparently non-pathogenic parvovirus that depends on certain other viruses to supply gene products, known as helper factors, essential to its own replication, a quirk of biology that has made AAV well-suited to serve as a recombinant vector. For example, adenovirus (AdV) can serve as a helper virus by providing certain adenoviral factors, such as the E1A, E1B55K, E2A, and E4ORF6 proteins, and the VA RNA, in cells co-infected by adenovirus and AAV. Other helper viruses, such as herpes simplex virus, have been identified as well. The dependence of AAV replication on accessory factors supplied by other viruses led AAV to be characterized as a type of dependovirus. AAV virions have two major structural features, called the capsid and genome, respectively. The capsid is an icosahedral protein shell that encloses and protects (encapsidates) the viral genome, which contains genes and other sequences required for viral replication in infected cells.

The AAV genome is a single strand of DNA containing two genes called rep and cap. In AAV2, a naturally occurring AAV that infects humans and is particularly well characterized biologically, the genome is about 4.7 kilobases long. By virtue of alternative splicing of the transcripts from two promoters, the rep gene is capable of producing four related multifunctional proteins called Rep (called Rep 78, Rep 68, Rep 52 and Rep 40 in AAV2, named according to their apparent molecular weights) that are involved in viral gene expression, and replication and packaging of genomes. Alternative splicing of the transcript from the single promoter controlling the single cap gene produces three related structural proteins, VP1, VP2, and VP3, a total of 60 of which self-assemble to form the virus's icosahedral capsid in a ratio of approximately 1:1:10, respectively. VP1 is longest of the three VP proteins and contains amino acids in its amino terminal region that are absent from VP2, which in turn is longer than VP3 and contains amino acids in its amino terminal region that are absent from VP3. In addition to containing the genome, capsid proteins mediate specific binding interactions with receptors on the surface of target cells, based on which AAV can be restricted in their ability to infect certain animal species, and even tissues within the same type of animal, a phenomenon called tropism. For example, one type of AAV may preferentially infect liver cells (e.g., hepatocytes) as compared to muscle or neuronal cells.

In addition to the rep and cap genes, intact AAV genomes have a relatively short (145 nucleotides in AAV2) sequence element positioned at each of their 5′ and 3′ ends called an inverted terminal repeat (ITR). ITRs contain nested palindromic sequences that can self-anneal through Watson-Crick base pairing to form a T-shaped, or hairpin, secondary structure. In AAV2, ITRs have been demonstrated to have important functions required for the viral life cycle, including converting the single stranded DNA genome into double stranded form required for gene expression, as well as packaging by Rep proteins of single stranded AAV genomes into capsid assemblies.

Numerous naturally occurring types of AAV have been discovered in different species. At one time, only six types of primate AAV had been isolated from biological samples (AAV1, AAV2, AAV3, AAV4, AAV5, and AAV6), the first five of which were sufficiently distinct structurally to be classified as different serotypes based on antibody cross reactivity experiments. Later, two novel AAVs, called AAV7 and AAV8 were discovered by PCR amplification of DNA from rhesus monkeys using primers targeting highly conserved regions in the cap genes of the previously discovered AAVs (Gao, G. et al., Proc. Natl. Acad. Sci. USA, 99:11854-9, 2002). Subsequently, a similar approach was used to clone numerous novel AAVs from human and non-human primate tissues, vastly expanding the scope of AAV capsid protein sequences (Gao, G. et al., J. Virol., 78:6381-8, 2004). Many AAV capsid protein sequences are highly similar to each other, or previously identified AAVs, and while often referred to as distinct AAV “serotypes,” not all such capsids would necessarily be expected to be immunologically distinguishable if tested by antibody cross reactivity. AAVs, or AAV capsids, which are not serologically distinguishable from a defined serotype but contain capsid proteins with a different amino acid sequence are better termed variants of the known serotype. Numerous capsids made from naturally and non-naturally occurring capsid proteins have found utility in creating AAV gene therapy vectors.

As established studying AAV2, after binding one or more receptor molecules on a cell surface, the AAV viral particle enters the cell via endocytosis. Upon reaching the low pH of lysosomes, capsid proteins undergo a conformational change that allows the capsid to escape into the cytosol and then be transported into the nucleus. Once there, the capsid disassembles, releasing the genome that is acted on by cellular DNA polymerases to synthesize the second DNA strand starting at the ITR at the 3′ end, which functions as a primer after self-annealing. Expression of the rep and cap genes can then commence, followed by formation and release from the cell of new viral particles.

### AAV Vectors

The relative simplicity of AAV structure and life cycle, and the fact that it is not known to be pathogenic in humans, inspired researchers to engineer AAV and investigate if it could be converted from a virus to a recombinant vector for gene therapy. Briefly, this was done by cloning the entire genome of AAV2, including both ITRs, into a plasmid, removing the rep and cap genes into a separate plasmid, and replacing them with a heterologous gene expression cassette comprising a promoter controlling a protein encoding transgene. Thus, the only viral genomic sequences retained in the vector were the ITRs, due to their critical function in packaging and gene expression, and without which AAV vectors could not be produced or function to express the transgene after transduction of target cells. Finally, to avoid the need for co-infection with a helper virus, necessary for replication of AAV virions, genes for the so-called helper factors (such as, in the case of AdV, the E1A, E1B55K, E2A, E4ORF6, and VA RNA helper factors) were cloned into a third plasmid.

When the three plasmids (which are sometimes called the transgene, rep/cap, and helper plasmids) were transfected together into mammalian host cells, Rep and capsid proteins, and the helper virus factors were expressed from their respective plasmids. These gene products then functioned in the host cells to replicate the vector into single stranded DNA from the plasmid on which its sequence resided, assemble capsids, and package the single stranded genomes into the capsids, forming vectors. The vectors could then be purified from the host cells. Because the rep and cap genes existed in trans on a different plasmid, outside their usual context flanked by ITRs, they were not packaged into the vectors. Consequently, while AAV vectors produced this way were able to bind to and convey the expression cassette within their genomes into target cells, they are unable to replicate and create new vector particles.

If vectors function as intended, after transduction, the expression cassette will be transcriptionally active and produce the gene product encoded by the transgene in the target cell. AAV vectors are highly versatile because vectors comprising a variety of transgenes under the control of different functional sequences and regulatory elements in various configurations can be designed and paired with a variety of naturally occurring and engineered capsids, with different tropisms and other properties. Many types of gene products can therefore be produced, with a degree of control over the types of cells that are transduced and amount of gene product that is made.

AAV vectors comprise a vector encapsidated by an AAV capsid. In some embodiments, the AAV vector comprises at least one AAV inverted terminal repeat (ITR) and a heterologous nucleotide sequence with a desired function when present or expressed in a transduced target cell. In some embodiments, the heterologous nucleotide sequence originates from a different type of virus, or an entirely different type of organism, such as an animal, plant, protist, fungus, bacteria, archaea, or other type of organism. In some embodiments, the heterologous nucleotide sequence replaces some or all of the native AAV rep and/or cap genes so that the vector is incapable of expressing functional Rep or VP proteins in transduced target cells. In some embodiments, the entire sequence of the vector consists of heterologous nucleotide sequences except for AAV inverted terminal repeat sequences positioned at the ends of the genome.

The length of a genome of the AAV vectors of the disclosure, inclusive of ITRs, can be any suitable length, which typically, but not necessarily, will not exceed the average genome size packaging capacity of the particular AAV capsid, which may be selected in the design and production of a particular AAV vector. Accordingly, in some non-limiting embodiments, the length of a genome of an AAV vector of the disclosure, inclusive of ITRs, can be at least or about 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, or 5200 nucleotides (or basepairs when the genome sequence is embodied in a plasmid for vector production), or an integer value between or range encompassing any of the foregoing specifically enumerated values.

### Expression Cassettes

In some embodiments, the heterologous nucleotide sequence comprises or consists of an expression cassette comprising a transgene operably linked with a promoter and optionally one or more enhancers, serving to control transcription initiation of the transgene from DNA into RNA, as well as a transcription termination element, such as a polyadenylation signal sequence, serving to terminate transcription of the transgene into RNA. AAV vectors can comprise more than one transgene, either as part of one transcriptional unit, or each being part of its own transcriptional unit. As described in later sections, expression cassettes can further comprise additional sequence elements designed to influence transcription, transcript stability, translation, or other functions.

As AAV vectors are typically designed, the structure of the expression cassette, and the genome overall, is limited by the packaging capacity of the capsid, so that the length of the transgene when combined with all other elements in the genome required for vector function, such as the transcriptional control elements and ITRs, does not exceed approximately 5 kilobases in the case of AAV2, although other types of capsids may have greater or smaller packaging limits. Within the size constraints, however, there is great flexibility in choice of transgenes, ITRs, and the other elements required for the vector to function for its intended purpose.

For purposes of gene therapy, the transgene can be any gene, the product of which would be understood to prevent or treat, although not necessarily cure, any disease, disorder or condition of a subject in need of prevention or treatment. In some embodiments, gene therapy is intended to prevent or treat a disease, disorder or condition characterized by an abnormally low amount or even absence of a product produced by a naturally occurring gene in a subject, such as might occur due to a loss of function mutation. Relating to such embodiments, the transgene can be one intended to compensate for the subject's defective gene by providing to at least some of the subject's cells the same or similar gene product when expressed. A non-limiting example would be a vector designed to express a functional version of clotting factor IX for use in gene therapy of hemophilia B, which is caused by a loss of function mutation in the native factor IX gene. In other embodiments, however, the transgene could be one intended to counteract the effects of a deleterious gain of function mutation in targeted cells. In some embodiments, the transgene can encode a transcriptional activator to increase the activity of an endogenous gene that produces a desirable gene product, or conversely a transcriptional repressor to decrease the activity of an endogenous gene that produces a deleterious gene product.

In some embodiments, the transgene can encode for a polypeptide, or code for an RNA molecule with a function distinct from encoding protein, such as a regulatory non-coding RNA molecule (e.g., micro-RNA, small interfering RNA, piwi-acting RNA, enhancer RNA, long non-coding RNA, etc.). Protein encoding sequences in a transgene can be codon-optimized, and translation start sites (e.g., Kozak sequence) can be modified to increase or decrease their tendency to initiate translation. In some embodiments, a transgene encoding amino acid sequence can contain one or more open reading frames, and/or contain one or more splice donor and acceptor site pairs to permit alternative splicing of different messages and polypeptide sequences from such messages. Transgenes encoding proteins further comprise one or more stop codons to end translation of the polypeptide chain.

In some embodiments, a vector can be designed for purposes of editing or otherwise modifying the genome of a target cell. For example, a vector can include an expression cassette or transgene flanked by homology arms intended to promote homologous recombination between the vector and the target cell genome. In another example, a vector can be designed to carry out CRISPR gene editing by expressing a guide RNA (gRNA) and/or an endonuclease, such as Cas9 or related endonucleases, such as SaCas9, capable of binding the gRNA and cleaving a DNA sequence targeted by the gRNA.

### PGRN Transgenes

In some embodiments, AAV vectors of the disclosure comprise a vector comprising an expression cassette comprising a coding sequence (transgene) for a progranulin protein (abbreviated “PGRN”), or variant thereof, including a human progranulin protein, or variant thereof. In some embodiments, the progranulin protein is identical to the 593 amino acid long 88 kDa human progranulin precursor protein (NCBI Reference Sequence: NP_002078.1 or SEQ ID NO:16), which includes a 17 amino acid long signal peptide (SEQ ID NO:18) and 576 amino acid long mature granulin polypeptide (SEQ ID NO:18). In some embodiments, after cleavage of the signal peptide, the mature granulin polypeptide is further cleaved into a variety of approximately 6 kDa peptide, which have pleotropic functions depending on the cellular or organismal context. In other embodiments, the expression cassette comprises coding sequence for a non-human progranulin protein.

In yet other embodiments, the PGRN protein can include any naturally occurring variants of human PGRN protein that do not contain pathogenic mutations, such as premature translation termination codons, or amino acid substitutions, insertions or deletions that substantially impair PGRN activity, and/or protein stability. In yet further embodiments, the PGRN protein can include engineered variants of human PGRN protein that retain PGRN activity, such as the chimeric variants and variants with amino acid substitutions, insertions or deletions designed to modulate PGRN activity, add or remove glycosylation sites, add or remove or change internal cleavage sites for the granulins that are ordinarily cleaved from the mature granulin, or sites for other post-translational modifications, or alter other aspects of PGRN structure or function. In some embodiments, the native signal peptide sequence is modified or replaced entirely with a signal peptide sequence from a similar or entirely different secreted protein.

In certain embodiments, the PGRN protein variant is a carboxy-terminal truncation variant in which one or more amino acids ordinarily present in the full-length wild-type human progranulin are deleted, such as 1, 2, 3, 4, 5, 6, 7, 8, 9, 10, 11, 12, 13, 14, 15, 16, 17, 18, 19, or 20, or more amino acids are deleted from the progranulin carboxy-terminus compared to the full-length wild-type human progranulin protein amino acid sequence (such as that provided by SEQ ID NO:16). In some embodiments, the PGRN protein variant has deleted the final 3 carboxy-terminal amino acids (QLL) otherwise present in full-length wild-type human PGRN protein and has the amino acid sequence of SEQ ID NO:14. The latter PGRN protein variant is sometimes referred to herein as “PGRNΔ3” or “PGRNDel3”. In some embodiments, PGRN protein variants, such as PGRNΔ3, have reduced or no specific binding to the receptor protein known as sortilin 1 (SORT1), as compared to full-length wild-type PGRN protein. In some embodiments, the PGRNΔ3 variant undergoes further post-transcriptional and post-translational modification after delivery by the viral vector. Additional terminal cleavages are known to occur, for example, including a carboxy-terminal single amino acid cleavage resulting in a PGRNΔ4 protein variant. Other known post-transcriptional and post-translational processing events can further modify the PGRNΔ3 vector provided protein, including, but not limited to, glycosylation, isomerization, full or partial degradation, cleavage of amino acid sequence, e.g., cleavage of one or more signal sequences, addition of molecular, e.g., peptide “tags” or functional or signaling sequences, and the like.

In certain other embodiments, the PGRN protein variant comprises at least one amino acid substitution mutation compared to the full-length wild-type human progranulin protein amino acid sequence (such as that provided by SEQ ID NO:16), such as 1, 2, 3, 4, 5, 6, 7, 8, 9, 10, 11, 12, 13, 14, 15, 16, 17, 18, 19, or 20, or more amino acids are changed from their wild-type counterpart. In some of these embodiments, a substitution mutation may be a conservative amino acid substitution, in which an amino acid ordinarily present is substituted by another amino acid with an R group having similar physico-chemical and/or or size characteristics. Alternatively, in other embodiments, a substitution mutation may be a non-conservative amino acid substitution, in which an amino acid ordinarily present is substituted by another amino acid with an R group having non-similar physico-chemical and/or or size characteristics.

For use in AAV vectors of the disclosure, the nucleotide sequence encoding the PGRN protein can be any nucleotide sequence capable of encoding the desired PGRN protein in the type of cell desired to be transduced by the vector, such as a neuron, In some embodiments, the nucleotide sequence encoding PGRN protein (i.e., the transgene) is the same as exists in a naturally occurring gene encoding PGRN (i.e., the exons of such gene), or is the DNA sequence that corresponds to the mRNA sequence transcribed from such gene. In some embodiments, wherein the PGRN protein is full-length wild-type human progranulin protein, the encoding nucleotide sequence is provided by nucleotides 41 to 1822 of NCBI Reference Sequence: NM_002087.4, inclusive of the stop codon, or by SEQ ID NO:15. In some embodiments, wherein the PGRN protein is the PGRNΔ3 protein variant, the encoding nucleotide sequence is provided by SEQ ID NO:8.

In other embodiments, the nucleotide sequence encoding PGRN protein can differ at one or more nucleotide positions compared to a naturally occurring nucleotide sequence and, by virtue of the redundancy in the genetic code, still encode the identical PGRN protein as the naturally occurring gene sequence, or PGRN protein variant that, but for the differences in the polypeptide relative to wild-type PGRN, is otherwise encoded by the naturally occurring gene sequence. In some embodiments, the nucleotide sequence encoding PGRN protein can be intentionally modified to affect its function in transduced cells, such as to eliminate sequence motifs capable of stimulating an innate immune response, to eliminate cryptic splice junctions, to eliminate alternative start codons, to increase the stability of the corresponding mRNA, and/or to increase the rate of translation of mRNA into protein. In other embodiments, the nucleotide sequence encoding PGRN protein can be intronless, or can include one or more introns interrupting the coding sequence, but that are removed by the splicing apparatus in transduced cells so as to allow translation of the desired PGRN protein.

In some embodiments of the AAV vectors of the disclosure, the transgene comprises a protein sequence that is highly similar to, or identical with the protein sequence encoded by a certain nucleotide reference sequence, but where the nucleotide sequences of the transgene and reference sequence are not identical, but rather share a certain percent identity, the differences corresponding to positions within codons that do not change the corresponding amino acid (i.e., are silent changes). For example, in some embodiments, the transgene comprises or consists of sequence that encodes the same full-length PGRN protein as SEQ ID NO:15 and has a nucleotide sequence at least 50%, 55%, 60%, 65%, 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% identical to SEQ ID NO:15; or comprises or consists of sequence that encodes the same PGRNΔ3 protein variant as SEQ ID NO:8 and has a nucleotide sequence at least 50%, 55%, 60%, 65%, 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% identical to SEQ ID NO:8.

The percentage of nucleotide sequence identity between a reference sequence and a transgene can be determined by any method known in the art. For example, in some embodiments, the nucleotide sequences of the reference sequence and the transgene (or the amino acid sequences encoded by them) can be aligned and compared over their entire lengths and a percent nucleotide sequence identity calculated using a computer algorithm. An exemplary algorithm for globally aligning and comparing nucleotide sequences is the Needleman-Wunsch algorithm. In other embodiments, however, a local alignment algorithm, such as the BLAST algorithm can be used (Needleman, S. & Wunsch, C., J. Mol. Biol., 48:443-53, 1970; States D. et al., Methods, 3:66-70, 1991; Pearson, W., Curr. Protoc. Bioinformatics, 43:3.5.1-3.5.9, 2013). In some embodiments, where one or the other of the reference and transgene sequences contains non-coding sequence, such as an intron or a stop codon, then the non-coding sequence(s) are ignored and only the protein coding sequence within the reference and transgene sequences are aligned and compared. Once the optimal global alignment between a reference sequence and a transgene is established, the percent of identical nucleotides between the aligned sequences can be calculated.

As is known in the art, sequence comparison algorithms can allow users to define substitution scores and gap penalties, parameters used to calculate alignment scores for the numerous possible alignments that can be made. The alignment with the highest score is then considered optimal. The substitution score involves assigning a numerical reward for matches and penalty for mismatches. Exemplary sets of respective match and mismatch scores include 1,−1; 1,−2; 1,−3; 1,−4; 2,−3; 4,−5, although others are possible. The gap cost involves assigning a numerical penalty for existence of a gap (insertion or deletion of a nucleotide) as well as penalty for extending the width of the gap once formed. Increasing the gap costs will result in alignments that decrease the number of gaps introduced. Exemplary sets of respective costs for gap existence and extension include 0,−4;−2,−2;−2,−4;−3,−3;−4,−2;−4,−4;−5,−2;−6,−2, although others are possible. In some embodiments, the alignment and comparison of the reference and transgene sequences is carried out using the default substitution scores and gap penalties, and any other default settings, provided with computer software or algorithm for performing the analysis.

### Signal Peptide Sequences

In some embodiments, AAV vectors of the disclosure comprise a vector comprising an expression cassette comprising a coding sequence (transgene) for a progranulin protein comprising a mature granulin polypeptide sequence, but in which the signal peptide naturally present in the wild-type human progranulin protein is replaced with a signal peptide from a different secreted polypeptide from human or another species to (i.e., a heterologous signal peptide sequence), for example, improve the rate at which PGRN (or variant thereof) made in transduced cells is secreted, or for some other reason, such as reduced immunogenicity. In some embodiments, the mature granulin polypeptide comprises or consists of the amino acid sequence of SEQ ID NO:18, or a carboxy-terminal truncation thereof lacking the last 3 amino acids (QLL).

Any signal peptide sequence known in the art to be effective to cause a protein to be secreted from a cell in which it is synthesized may be used in connection with the AAV vectors of the disclosure. In some embodiments, such signal peptides are removed from the protein by the cell in the process of secretion. Numerous heterologous secretion signal peptides are known in the art and can be used to facilitate secretion of PGRN (or variant thereof) from cells, such as neurons or other brain cells, transduced with AAV vectors of the disclosure.

In some embodiments, the signal peptide sequence may originate or be derived from any of a variety of proteins made by and secreted from neurons or other cells in the central or peripheral nervous system. Non-limiting examples include signal peptide sequences from human proteins such as growth hormone, proenkephalin A, beta-neoendorphin-dynorphin, neuroendocrine protein 7B2, prolactin, gastrin-releasing peptide II, secretogranin I, growth hormone releasing factor, axonin I, neuroendocrine convertase 2, vasopressin copeptin, with many others being known in the art. The amino acid sequences and encoding nucleotide sequences for these and other secreted proteins are available in public sequence databases, such as Genbank. The amino acid sequence of naturally occurring signal peptides can be modified to desirably alter their function, as can the nucleotide sequence encoding such wild-type or modified signal peptides to achieve a desired type of sequence optimization, such as removal of CpG motifs. In yet other embodiments, entirely synthetic secretion signal peptide sequences can be used.

### Transcription Control Regions

AAV vectors of the disclosure intended to express PGRN protein in and/or from transduced cells can further comprise, as part of the vector, one or more transcription control regions in operable linkage with the transgene encoding the PGRN polypeptide sequence. As discussed further below, different types of transcription control regions are known in the art that can be used to control initiation of transcription of the transgene into RNA. As used herein, the term “operable linkage,” and variations such as “operative linkage,” “operably linked,” and “operatively linked,” refers to a functional relationship between the transcription control region and transgene, so that the control region can affect transcription of the transgene (whether positively or negatively), without specifying any particular spatial or structural relationship between them. Thus, for example, a transcription control region could be operably linked with a transgene even though it is positioned 5′ or 3′ of the transgene, and/or positioned immediately adjacent to or distal from the transgene. Transcription control regions can be constitutively active, active in specific cells or tissues, inducibly active in response to some environmental stimulus, be derived from a naturally occurring gene (of any suitable species) and can be modified to improve or change its function, or even be entirely synthetic.

In some embodiments, a transcription control region comprises a promoter region, which comprises the minimal DNA sequence required to initiate transcription by the transcription apparatus in transduced cells (e.g., a TATA box or initiator sequence), often as well as one or more additional proximal elements that act singly or cooperatively to increase the rate of transcription from the basal promoter. Depending on its sequence, a promoter can initiate transcription by RNA polymerase I, II, or III, but promoters from protein encoding genes, which are usually transcribed by RNA pol II, are often used in AAV vectors intended to express a polypeptide, such as PGRN or variant thereof, such as PGRNΔ3, in transduced cells.

In other embodiments, a transcription control region comprises or further comprises at least one enhancer region, which functions to further increase the rate of gene transcription beyond what the basal promoter alone can sustain. In their natural context, enhancers are often positioned distally from the promoter of the gene on which they act, sometimes tens to hundreds or thousands of basepairs upstream (i.e., 5′), but enhancers can also occur elsewhere, such as in introns or downstream (i.e., 3′) of the gene on which they act. While promoter regions may contain proximal enhancer elements (subsequences that, if removed, would reduce transcription from the basal promoter), enhancers do not usually contain sequences that can function as a basal promoter. Although in nature enhancer regions are often positioned distally to the promoter of the gene on which they act, enhancer regions, or enhancer elements from within larger enhancer regions (such elements often corresponding to DNA binding sites for transcription factors), can sometimes retain at least some of their transcription enhancing function when removed from their natural context and repositioned much closer to a promoter, whether from the same or even a different gene.

Enhancer and promoter regions of genes described in the scientific literature may be too large to be accommodated by the packaging capacity of AAV capsids when combined with a transgene and other genomic elements required for vector function. Accordingly, in some embodiments, functional subsequences within longer enhancer or promoter regions can be identified using methods familiar to those of ordinary skill, and the shorter functional subsequences incorporated into transcription control regions for use in the vectors of the disclosure. In this manner, the size of transcription control regions can be reduced while maintaining their desired function. Using this approach functional elements from naturally occurring enhancers or promoters can be combined in novel ways, such as by modifying their number, spacing and/or arrangement, to create hybrid or synthetic enhancers and/or promoters with improved properties. In some embodiments, the enhancer and promoter can each be derived from the same, naturally occurring gene, whereas in other embodiments, the enhancer and promoter can originate from entirely different genes, including genes of different species.

In some embodiments, from the perspective of a coding strand (i.e., plus strand) single stranded DNA AAV vector, a promoter sequence is positioned 5′ of a downstream sequence to be transcribed into RNA, such as a transgene encoding a protein, such as PGRN or variant thereof, such as PGRNΔ3. In some embodiments, an enhancer element or region, if present, can be positioned 5′ of the promoter sequence, or instead be positioned elsewhere in the genome, such as in a 5′ or 3′ untranslated region (UTR) adjacent the transgene, in an intron, 3′ of a transcription termination signal sequence, or elsewhere. In some embodiments, a vector can comprise more than one enhancer region (of same or different types), which can be positioned adjacent to each other, or spaced apart, and/or separated by other functional elements within the genome. In some embodiments, the same enhancer element or region is provided in a tandemly arranged array of repeating units, such as 2, 3, 4, or more.

In some embodiments, transcription control regions for use in the AAV vectors of the disclosure are non-tissue specific, meaning that they are constitutively active in many different cell types, although not necessarily all. According to some embodiments, non-tissue specific transcription control regions include promoters (which may include enhancer elements proximal to a basal promoter) derived from certain viruses, such as the human cytomegalovirus major immediate early gene (CMV-IE) (Boshart, M. et al., Cell, 41:521-30, 1985; Yew, N. et al., Hum. Gene Ther., 8:575-84, 1997); simian virus 40 (SV40); as well as the retroviral long terminal repeat (LTR) promoters from Rous sarcoma virus (RSV) and Moloney murine leukemia virus (MoMLV). In other embodiments, non-tissue specific transcription control regions include promoters (which may include proximal enhancer elements) can be derived from genes active in many different cell types (which are sometimes referred to as “housekeeping” genes), including from different types of animals, such as the human polypeptide chain elongation factor (EF1α) gene; the phosphoglycerate kinase (PGK) gene; the ubiquitin C (UbiC) gene; the chicken beta-actin (CBA) gene; the U1a1 or U1b2 small nuclear RNA promoters (Bartlett, J. et al., Proc. Natl. Acad. Sci. USA, 93:8852-7, 1996; Wu, Z. et al., Mol. Ther., 16:280-9, 2008); the histone H2 or histone H3 promoters (Hurt, M. et al., Mol. Cell Biol., 11:2929-36, 1991).

Likewise, enhancer regions can be derived from viruses and genes active in different cell types from different types of animals. As noted, in some embodiments, a promoter and enhancer derived from the same gene can be combined to create a transcription control region for use in the vectors of the disclosure, but enhancers and promoters from different genes can be combined to create hybrid transcription control regions. A commonly used example is the 1.6 kilobase hybrid enh/pro region called CAG (or CAGGS) comprising the CMV immediate-early enhancer, the chicken beta actin (CBA) gene promoter and the CBA intron/exon 1 (Niwa, H. et al., Gene, 108:193-9, 1991); Ikawa, M. et al., Dev. Growth Differ., 37:455-9, 1995), and later modifications that reduced its size, including one in which the CBA intron was replaced with a smaller simian virus 40 (SV40) intron (Wang, Z. et al., Gene Ther., 10:2105-11, 2003), and another called CBA hybrid intron (CBh) that replaced the SV40 intron with a hybrid intron composed of a 5′ donor splice site from the CBA 5′ UTR and a 3′ acceptor splice site from the MVM intron (Gray, S. et al., Hum. Gene Ther., 22:1143-53 2011).

In some embodiments, transcription control regions for use in the AAV vectors of the disclosure can be central nervous system (CNS) or brain tissue specific, meaning that they are more or most active in directing expression of a transgene in cell types within the CNS or brain, compared to cells of other tissues or organs, such as the muscle or liver. In some embodiments, the CNS or brain cell types in which transcription control regions of AAV vectors of the disclosure are preferentially active include, without limitation, neurons, glial cells (such as microglial cells, astrocytes, and oligodendrocytes), and ependymal cells, with other cell types being possible. Without wishing to be bound by any particular theory of operation, one mechanism by which brain tissue or neuronal (or any other cell type in the brain) gene transcriptional specificity may occur is the presence in an enhancer and/or promoter of one or more specific binding sites for DNA binding transcriptional activator proteins preferentially expressed in brain cells, such as neurons or other cell types in the brain. Use of a brain tissue or neuron (or other brain cell type) specific transcription control region can be advantageous, in some embodiments, by reducing or even preventing transgene expression in cells outside of the brain or non-neuronal cells (or other brain cell type) that may be transduced by a vector, which can desirably reduce the risk of off-target effects.

Certain brain or neuronal (or other brain cell type) specific genes expressed at high level have both enhancers and promoters that may be included in transcription control regions of the AAV vectors of the disclosure. In some embodiments an enhancer and a promoter derived from the same gene may be combined in a brain or neuronal (or other brain cell type) specific transcription control region, whereas in other embodiments, an enhancer from one gene and a promoter from a different gene may be combined in a hybrid brain or neuronal (or other brain cell type) specific transcription control region. When derived from the same gene, a transcription control region sequence comprising one or more enhancers and promoter may be copied as it exists in the native gene context from which it is derived, or engineered to reduce its length, such as by deleting non-transcriptionally active sequences separating the one or more enhancers and the promoter. In some embodiments, enhancers and promoters for use in brain or neuronal (or other brain cell type) specific transcription control regions may be derived from genes of different species. In yet other embodiments, the sequence of an enhancer and/or a promoter in a transcription control region can be modified relative to its original sequence by changing, adding or removing nucleotides to improve its function, such as increasing transcription activator binding, reducing transcription repressor binding, or reducing the size of the transcription control region.

In some embodiments, it is the enhancer that provides for brain or neuronal (or other brain cell type) specific expression and the promoter is not itself brain or neuronal (or other brain cell type) specific, whereas in other embodiments, it is the promoter that provides for brain or neuronal (or other brain cell type) specific expression and the enhancer, if present, is not itself brain or neuronal (or other brain cell type) specific but is capable of increasing the rate of transcription from the brain or neuronal (or other brain cell type) specific promoter. For example, a strong viral enhancer, such as the human CMV major immediate early gene enhancer, could be paired with a brain or neuronal (or other brain cell type) specific promoter, or a strong brain or neuronal (or other brain cell type) specific enhancer, such as from the synapsin 1 gene, could be paired with a strong viral promoter, such as the SV40 early promoter. In other embodiments, both the enhancer and promoter each are brain or neuronal (or other brain cell type) specific. In some embodiments, different enhancer regions can be combined to form chimeric enhancer regions that are used in transcription control regions in AAV vectors of the disclosure.

Brain tissue or neuron (or other brain cell type) specific transcription control regions (whether an enhancer, a promoter, or both) for use in the AAV vectors of the disclosure can be derived from genes that are naturally expressed at high levels in brain or neurons (or other brain cell type), or even specific regions of brain, or subtypes of neurons. For example, and without limitation, the synapsin 1 gene (SYN1), the neuron specific enolase (NSA) gene, and the tubulin al gene each contain a neuron-specific promoter, the glial fibrillary acidic protein (GFAP) gene promoter is at least partly astrocyte-specific, the L7-6 gene promoter is at least cerebellar Purkinje cell-specific, the Ca2+/calmodulin-dependent protein kinase II (CaMKII) gene promoter is at least partly specific for forebrain excitatory neurons, the distalless homeobox (DLX) gene enhancer is at least partly specific for forebrain inhibitory neurons, the glutamic acid decarboxylase (GAD) 65 gene promoter is also at least partly specific for inhibitory neurons, the tyrosine hydroxylase gene promoter is at least partly specific for catecholaminergic neurons, and the transcription control regions (promoter and/or enhancer) of the following genes are at least partly specific for neurons: ADORA2A, ATP6V1C2, AVP, C8ORF46, CARTPT, CCKBR, CCL27, CD68, CLDN5, CRH, CX3CR1, DBH, DCX, DDC, DRD1, FEV, FEZF2, GABRA6, GAL, GCHFR, GFAP, GPR88, GPX3, GRP, HAP1, HBEGF, HCRT, HSPA12B, HTR1A, ICMT, LCT, MKI67, NR2E1, NTSR1, OLIG1, OXT, PCP2, PITX3, PKP2, POGZ, RAMP3, RGS16, RLBP1L2, S100B, SLC6A2, SLC6A3, SLC6A4, SLC6A5, SLC7A5, SLITRK6, TAC1, TAC3, TBR1, THY1, TNNT1, TRH, UGT8, VIM, and VIP.

In some embodiments, a transcription control region of AAV vectors of the disclosure to express PGRN protein, or variant thereof such as PGRNΔ3, in transduced neurons can comprise or consist of the promoter region of the human synapsin 1 gene (SYN1), which has been demonstrated to confer neuronal-cell specific transcriptional regulation, or subsequence thereof that retains such neuron specific transcriptional regulation. The promoter region of the human synapsin 1 gene is described in further detail in, e.g., Thiel, G, et al., Characterization of tissue-specific transcription by the human synapsin I gene promoter, PNAS 88:3431-3435 (1991), Schoch, S, et al., Neuron-specific Gene Expression of Synapsin I, JBC 271 (6): 3317-3323 (1996). In some embodiments, a transcription control region can comprise or consist of, or further comprise, a human synapsin 1 gene (SYN1) promoter that, in some embodiments, comprises, consists essentially or, or consists of, the nucleotide sequence of SEQ ID NO:6, or a neuron-specific transcription functional subsequence thereof.

A variety of brain or neuronal (or other brain cell type) specific transcription control regions for use in gene therapy vectors (e.g., AAV, adenoviral or lentiviral vectors) have been created by adapting enhancers and/or promoters from brain or neuronal (or other brain cell type) specific genes, for example by reducing their length, any of which can be used in the AAV vectors of the disclosure to express PGRN protein, or variant thereof such as PGRNΔ3, in transduced cells in the brain, such as neurons, or other brain cell type. Non-limiting examples of such brain or neuronal (or other brain cell type) specific transcription control regions, including transcriptional functional portions of the human synapsin 1 gene promoter (Kügler, S. et al., Mol. Cell Neurosci., 17:78-96, 2001; Kügler, S. et al., Gene Ther., 10:337-47, 2003; Hioki, H. et al., Gene Ther., 14:872-82, 2007; Portales-Casamar, E. et al., Proc. Natl. Acad. Sci. USA, 107:16589-94, 2010; Jackson, K. et al., Front. Mol. Neuro., 9:1-11, 2016; Massaro, G. et al., Hum. Mol. Genet., 29:1933-49, 2020; Finneran, D. et al., Front. Neurol., 12:1-12, 2021; Radhiyanti, P. et al., Neurosci. Lett., 756:1-6, 2021; U.S. Pat. No. 7,341,847).

### Transcription Termination Signal Sequences

In some embodiments, AAV vectors of the disclosure comprise a vector comprising a transcription terminator sequence positioned, from the perspective of a coding (plus) strand single stranded DNA vector, 3′ of the transgene. In some embodiments, another sequence, such as 3′ untranslated region (UTR) sequence, can be positioned between the transgene sequence and the transcription terminator sequence (Proudfoot, N., Genes Dev., 25:1770-82, 2011; Kuehner, J. et al., Nat. Rev. Mol. Cell Biol., 12:283-94, 2011; Porrua, O & Libri, D., Nat. Rev. Mol. Cell Biol., 16:190-202, 2015).

In some embodiments, particularly when the transgene contains protein encoding-sequence (as opposed to the sequence of an RNA with some function other than encoding protein), the transcription terminator sequence can be a polyadenylation signal sequence (abbreviated variously as “polyA,” “pA,” “poly (A)” or “p (A)”). In some embodiments, pÅ signal sequences can be derived from naturally occurring genes and used in vectors, whereas in other embodiments, pA signals can be modified, such as by shortening them compared to their natural counterparts or altering their sequence to make them more efficient at transcription termination. In other embodiments, pA signals can be hybrid sequences, combining pA sequences from different genes, or synthetic.

Non-limiting examples of pA signals that may be used in the vectors of the disclosure include the pA signal from the bovine growth hormone gene (bGH pA); human, mouse or rabbit beta-globin gene; SV40 late gene; sNRP1; spA; herpes simplex virus thymidine kinase gene (HSV TK); or adenovirus type 5 L3 polyadenylation site, with others being possible. In other embodiments, transcription terminators for use in vectors of the disclosure include those that terminate RNA transcripts without directing polyadenylation, such as the histone H4 gene mRNA 3′ end processing signal (Whitelaw, E, et al., Nucleic Acids Res, 14:7059-70 (1986)).

In some embodiments, AAV vectors of the disclosure comprise vectors comprising a transgene, transcription of which is terminated by inclusion of a poly (A) site derived from the bovine growth hormone gene (bGH) that, in some embodiments, comprises or consists of the nucleotide sequence of SEQ ID NO:9 or SEQ ID NO:10; of a poly (A) site from SV40 virus that, in some embodiments, comprises or consists of the nucleotide sequence of SEQ ID NO: 26 or SEQ ID NO:27; or of a poly (A) site from the rabbit beta-globin gene that, in some embodiments, comprises or consists of the nucleotide sequence of SEQ ID NO:28.

### Other Vector Genomic Elements

In addition to transcription control regions and transcription termination signal, other sequences, including cis-regulatory elements, can be included in genomes of AAV vectors of the disclosure to improve, control or modulate transgene expression and/or translation in transduced cells, or confer other functions to the vectors. Such elements include, without limitation, untranslated regions from the 5′ and/or 3′ ends of genes, non-coding exons, introns, splice donor and acceptor sites, lox sites, internal ribosome entry sites (IRES), sequence encoding 2A peptides, elements that stabilize RNA transcripts, binding sites for regulatory miRNAs, micro RNA (miRNA) sequences, elements that enhance nuclear export of mRNAs, including viral post-transcriptional regulatory elements, such as the woodchuck hepatitis virus post-transcriptional regulatory element (WPRE), as well as any other element demonstrated empirically to improve transgene expression, even if the mechanism may be uncertain. In other embodiments, vectors can include so-called stuffer or filler sequences, which are intended only to increase the overall length of the vector to a desired size, for example, to achieve a length close to but still under the packaging capacity of a particular capsid, and thereby reduce the likelihood of adventitiously packaging truncated vectors or non-vector DNA into capsids.

Introns can, in some embodiments, be included in vectors to increase transgene expression and/or transcript stability. In some embodiments, a protein encoding transgene is provided in which the sequence of exons and intron(s) is the same as in the naturally occurring gene. In a gene possessing multiple exons and introns, however, one or more of the introns can be removed so as to minimize the overall length to facilitate the inclusion of other elements while not exceeding the capsid packaging capacity. In other embodiments, however, an intron can be provided from an entirely different gene than the gene providing the coding sequence for the vector transgene. Whether the intron is from the same or a different gene as the transgene, the intron can be modified from its original sequence, for example by changing certain nucleotides, or removing internal sequences to reduce its overall length while maintaining the splice donor and acceptor sequence motifs required for efficient splicing to occur, or other intronic cis elements important for function (for example, enhancers that may reside in the original unmodified intron sequence). Introns can also be hybrid, where the splice donor portion of the intron from one gene is paired with the splice acceptor portion of the intron from a different gene, or synthetic, with sequence that does not correspond to any known gene's intron. Introns, in some embodiments, can be positioned within, and therefore interrupt, the coding sequence of a transgene (and be provided with the donor and acceptor sites necessary for efficient splicing to occur), whereas in other embodiments an intron is present, but does not interrupt the protein coding sequence, and is instead positioned either 5′ or 3′ of the coding sequence. Where an intron does not interrupt coding sequence, it may be provided with some exonic sequence carried over from its original genetic context, so long as the exonic sequence does not contain a cryptic translation start signal. In some embodiments, an intron can be positioned 3′ of a promoter (from the perspective of plus strand ssDNA vector) and 5′ of the coding sequence. In other embodiments, an intron can be positioned distally from coding sequence in a vector, either upstream or downstream.

Non-limiting examples of introns that may be used in the AAV vectors of the disclosure include the small intron from the minute virus of mice (MVM) (Haut, D. & Pintel, D., J. Virol., 72:1834-43, 1998; Haut, D. & Pintel, D., Virology, 258:84-94, 1999); internally deleted intron 1 from human clotting factor IX (FIXm1 and FIXm2) (Kurachi, S. et al., J. Biol. Chem., 270:5276-81, 1995); chimeric beta globin splice donor and immunoglobulin heavy chain splice acceptor intron (GenBank U47120.2 nucleotides 890-1022); intron 1 from the mouse alpha globin gene; and the SV40 small t antigen intron that can comprise or consist of base pairs 4644 to 4552 of GenBank record J02400.1, and that can be modified at positions 4582 (g to c), 4580 (g to c), 4578 (a to c), and 4561 (a to t) (Nathwani, A. et al., Blood, 107:2653-61, 2006).

In some embodiments, post-transcriptional regulatory elements (PRE) can be included in vectors to increase transgene expression. Examples of PRE include the woodchuck hepatitis virus post-transcriptional regulatory element (WPRE), the hepatitis B virus post-transcriptional regulatory element (HPRE), and modifications thereof (Donello, J. et al., J. Virol., 72:5085-92, 1998; Loeb, J. et al., Hum. Gene Ther., 10:2295-305, 1999; Zanta-Boussif, M. et al., Gene Ther., 16:605-19, 2009; Patricio, M. et al., Mol. Ther. Nucleic Acids, 6:198-208, 2017; US Pub. Pat. Appl. 2018-0353620 A1). In some embodiments, from the perspective of a coding (plus) strand single stranded DNA vector, at least one WPRE sequence can be positioned downstream of a transgene (thus, 3′ of the stop codon for a transgene encoding a polypeptide) and upstream of the poly(A) signal sequence. In some embodiments, a plurality of PRE can be included, such as 2, 3, or more PRE, of the same or different type, which can be arranged in tandem. In some embodiments, AAV vectors of the disclosure comprise vectors comprising a WPRE element that, in some embodiments, comprises or consists of the nucleotide sequence of SEQ ID NO:29.

### AAV Inverted Terminal Repeats (ITRs)

Positioned at the termini of the adeno-associated virus genome are unique nucleotide sequences called inverted terminal repeats (abbreviated, “ITR”) that function as origins of viral DNA replication and as priming sites to support the conversion, in infected cells, of the single-stranded (ssDNA) genome into a double-stranded form (dsDNA) competent to support transcription of the rep and cap protein-encoding genes, also contained in the virus genome. The ITRs also function in packaging of replicated ssDNA genomes into AAV capsids. AAV ITRs contain multi-palindromic sequences that can fold back on themselves via intra-strand complementary base pairing to form dsDNA T-shaped hairpin secondary structures.

As explained further below, vectors of AAV vectors of the disclosure can include one or more AAV ITRs, which function similarly as they do in unmodified virus. Unless otherwise, use of the term “inverted terminal repeat” or “ITR” herein includes intact full-length ITRs, as well as ITRs with modified sequences (such as truncations, internal deletions (such as of a trs or D sequence), additions, and substitutions of one or more nucleotides) that retain one or more of the functions attributable to ITRs (even if less efficiently compared to an intact ITR of the same type), including but not limited to rescue of vector from recombinant DNA (such as a plasmid), vector replication, and/or packaging of vector into assembled capsids.

As they exist in packaged viral and vectors, the ITR positioned at the 3′ end of the ssDNA genome will have a free 3′ hydroxyl group, whereas the ITR positioned at the opposite 5′ end of the ssDNA genome will have a free 5′ end. The 5′ ITR can also referred to as the “left” ITR, and the 3′ ITR can also referred to as the “right” ITR. In the context of a plasmid, however, such as might be used in vector production, the vector sequence will exist in double-stranded form, such that there will be two sets each of 5′ ITRs and 3′ ITRs. To avoid ambiguity therefore, it should be specified which strand an ITR sits on to distinguish among them. In the absence of such specification, reference to the ITRs of a vector in double-stranded form, such as in a plasmid, is with respect to the plus or sense strand, i.e., the DNA strand on which the sequence of the transgene is the same as the coding sequence for a polypeptide product of the transgene, or of a functional RNA, where the transgene is not protein encoding.

In wild-type AAV2, the ITRs are 145 bases long, of which the terminal 125 bases comprise the palindromic subsequences. When annealed, the AAV2 ITR contains two double-stranded palindromes, B-B′ and C-C′, forming the arms of the hairpin, which are joined to a larger double-stranded palindrome, A-A′, forming the hairpin's stem. The ITR further contains a D sequence toward the non-terminal end of the ITR, which does not have a complementary sequence within the ITR and therefore remains single-stranded, but does have a complementary sequence in the D region of the ITR at the opposite end of the genome (thus, D and D′). The A-A′ stem structure includes a Rep-binding element (RBE) containing tetranucleotide repeat motifs to which the large AAV Rep proteins bind for purposes of introducing a sequence-specific and strand-specific nick at the terminal resolution site (trs) in the ITR sequence (between nucleotides 124 and 125 in the AAV2 ITR, counting from the 3′ end), a step required for DNA replication of the viral genome to occur.

When they renature, ITRs can fold into two configurations, called flip and flop, in which the sequence between the A and A′ inverted repeats is present as the reverse complement with respect to the other configuration. With respect to the 5′ ITR (left ITR), the order of terminal palindromic sequences for the flip configuration is 5′-ABB CC′A′D-3′, and the order for the flop configuration is 5′-ACC′BB′A′D-3′. With respect to the 3′ ITR (right ITR), the order of terminal palindromic sequences for the flip configuration is 3′-A′B′BC′CAD′-5′, and the order for the flop configuration is 3′-A′C′CB′BAD′-5′. Consequently, the flip configuration has the B′B palindrome closest to the free 3′ end, whereas the flop configuration has the C′C palindrome closest to the free 3′ end (Lusby, E. et al., J. Virol., 34:402-9, 1980; Srivastava, A. et al., J. Virol., 45:555-64, 1983; Samulski, R. et al., Cell, 33:135-43, 1983).

It is hypothesized that ITR secondary structure supports viral DNA replication by a self-priming single-strand displacement elongation mechanism initiated by endogenous cellular DNA polymerase at the ITR with the free 3′ hydroxyl group. Strand elongation leads to the formation of a monomeric dsDNA genome replicative intermediate with one covalently closed end. The duplex ITR at the open end refolds (isomerizes) into a double hairpin structure, forming a new 3′ ITR that is elongated while the complementary strand is displaced. The large AAV Rep proteins bind to the ITR at the closed end (downstream) and nicks the DNA at its terminal resolution site, initiating a second DNA replication complex that copies the downstream ITR before the DNA replication complex that initiated at the open end reaches it. The original replication complex displaces the opposite strand (whose ITR was just newly synthesized) and completes replication to what had been the closed end of the genome, now open with duplex ITRs available to isomerize into a double hairpin. Thus, the monomeric dsDNA genome replicative intermediate is recreated to start the cycle of replication over again, while the displaced ssDNA genome (whose 3′ ITR had been newly created) can be packaged into a virus particle.

The ssDNA genomes that are replicated will include both positive (plus, or sense) and negative (minus, or anti-sense) strand polarities, and evidence suggests that they are individually packaged into capsids with equal efficiency. Consequently, preparations of AAV vector particles, like the viruses from which they are adapted, can in some embodiments contain sense or antisense ssDNA genomes in about equal proportion. ITRs can also be modified, however, by selective removal of the D sequence from one of the two ITRs used to generate AAV vectors, which restricts packaging to either the negative or the positive strand of the vector (Wang, X-S, et al., J Virol, 70 (3): 1668-77 (1996)). Thus, in some other embodiments, a preparation of AAV vectors can contain vector particles in which most or substantially all the vectors are either positive stranded or negative stranded.

After infection, AAV virions are transported to the nucleus, where the ssDNA genomes are released from the capsid. Before the viral rep and cap genes can be expressed, it is hypothesized that the ssDNA genome must first be converted to dsDNA through complementary strand synthesis by cellular DNA polymerase initiating strand elongation at the 3′ ITR, a process that is believed to be slow and inefficient. It is also hypothesized that a faster mechanism may exist to form intracellular dsDNA genomes, in which complementary positive and negative ssDNA genomes originating from different virions infecting the same cell encounter each other in the nucleus and hybridize via intermolecular base pairing. Such duplex genomes could then support transcription without first requiring elongation by cellular DNA polymerase.

In designing AAV vectors, the only AAV viral DNA sequences retained in the vector are the ITRs because of their critical roles in DNA replication and packaging during production, and conversion of ssDNA genomes to dsDNA after transduction. The sequences encoding Rep and Cap proteins, and viral helper functions, which are also needed to produce vectors, can be provided in trans in a variety of ways known in the art. When the vector sequence, such as might be included in a plasmid used for AAV vector production, includes two intact ITRs, and is of a length that does not exceed the AAV capsid packaging capacity of ~5 kb, ssDNA genomes can be packaged, as noted above, but the requirement for dsDNA conversion can result in lower than desired transduction efficiency due to the inefficiency of that step before gene expression can occur. A strategy to overcome the requirement for dsDNA conversion by endogenous cellular DNA polymerase, and potentially improve transduction efficiency and expression of heterologous sequence, such as a therapeutic transgene, relies on replicating and packaging “self-complementary” AAV vectors (scAAV) that, because they contain positive and negative strand sequences in the same DNA molecule, can quickly renature to form a dsDNA transcriptional template through intramolecular base pairing (i.e., intramolecular hybridization) after capsid uncoating in the target cell nucleus.

Production of self-complementary genomes can be facilitated in at least two ways, both of which rely on failure of Rep to nick the ITR at the terminal resolution site during replication of the DNA molecule that is to become the genome. In the natural replication cycle of AAV, dimeric dsDNA genome replicative intermediate molecules can occur when Rep fails to nick the terminal resolution site of the downstream ITR of the monomeric genome replicative intermediate possessing the double hairpins at the open end and the single downstream ITR at the closed end. When this occurs, replication by the initial DNA replication complex (starting at the 3′ ITR at the open end) continues through the downstream (closed end) ITR as well as the displaced strand to form a dimeric dsDNA genome. This molecule is similar to the monomeric dsDNA genome replicative intermediate described above, but contains two genomes. Because it has an open end with duplex ITRs, it can isomerize to start a cycle of DNA replication as with the monomeric form. Alternatively, the closed end hairpin can undergo terminal resolution forming duplex ITRs that can isomerize so that DNA synthesis initiates from the resolved end. In either case, replication of the dimeric template generates a new dimeric dsDNA genome replicative intermediate, as well as displacing a ssDNA dimeric inverted repeat genome containing a 5′ ITR, viral genome sequence of one polarity, a central ITR, viral genome sequence of opposite polarity, and a 3′ ITR.

Ordinarily, the ssDNA dimeric inverted repeat viral genome will not be packaged because it exceeds the normal capsid packaging capacity. However, by designing vectors of reduced length so that ssDNA dimeric inverted repeat genomes, when formed, would not exceed the AAV packaging capacity, it is possible to produce vector particles containing self-complementary genomes. In practice, vector preparations produced this way can contain a mixture of particles in which are packaged one scDNA genome, or one or two monomeric ssDNA genomes, the proportions of all of which can vary between preparations.

After packaging, it is likely that scDNA genomes reside within capsids in single-stranded form (similar to ssDNA non-self-complementary genomes), but then rapidly anneal after capsid uncoating to form a dsDNA molecule with a covalently closed ITR at one end and two open ITRs at the other end, resembling the structure of a conventional viral genome after dsDNA conversion through self-priming. Thus, while not wishing to be bound by theory, a principal difference between so-called ssDNA and scDNA genomes is not the topology while encapsidated, but rather the topology each type of genome likely acquires after capsid uncoating and genome release within transduced cells.

A further modification, however, allows greater control over the production of vector particles containing scDNA genomes. Specifically, by mutating or deleting the terminal resolution site from one ITR, such as in the plasmid containing the vector sequence used for vector production, it is possible to inhibit or eliminate single-strand nicking at that ITR during the vector replication cycle. As a consequence, the replication complex initiated at the unmutated ITR progresses through the mutated hairpin and back to the initiating end, resulting in a dimeric dsDNA genome replicative intermediate, as in the case where terminal resolution of a wild-type ITR does not occur by chance. This intermediate, however, would contain a closed wild-type ITR at one end, mutated duplex ITR in the middle of molecule, and duplex open ITRs at the opposite end that are capable of isomerization. This molecule can then undergo normal rounds of replication and strand-displacement from the wild-type ITRs at each end, to produce displaced daughter genome copies containing a 5′ wild-type ITR, vector sequence of one polarity, mutated ITR in the middle, vector sequence of the opposite polarity, and a wild-type ITR at the 3′ end. If the heterologous sequence contains a protein-encoding transgene, the scDNA genome would contain both the coding sequence and its complement in the same DNA molecule. Although such genomes are believed to be packaged in single-stranded form inside capsids, they are also believed to be able to rapidly self-anneal into double-stranded form inside the nuclei of transduced cells due to the presence of significant amounts of self-complementary sequence outside the ITRs, in which form they can support transcription of the heterologous sequence. Production of scAAV from constructs containing mutated ITRs can yield more than 90% scDNA genomes.

In AAV2 capsids, which have a packaging capacity of approximately 4.7 kb, then excluding the ITR sequences, ssDNA genomes can accommodate approximately 4.4 kb of heterologous sequence, whereas scDNA vectors can accommodate about 2.2 kb. According to a particular non-limiting embodiment, the genome construct size (such as might be contained within a plasmid for AAV vector production) for producing scAAV vectors is about ^{~}2,500 nucleotides long, comprising a ^{~}2,200 nucleotide long heterologous sequence plus two ITRs (one wild-type and one mutated). This would result produce an scAAV genome ^{~}4,700 nucleotides long, which is below the typical AAV capsid packaging capacity.

ITR terminal resolution sites can be disrupted in various ways to facilitate production of scAAV vectors. For example, an exogenous sequence can be inserted into the terminal resolution site (trs) sequence itself, or into an adjacent sequence of the ITR, such as between the Rep binding element and the trs. Alternatively, the trs sequence could be deleted partially or in its entirety. In other embodiments, the adjacent D region can be deleted partially or in its entirety. In yet other embodiments, nucleotides within the trs can be substituted with different nucleotides that reduce frequency of trs nicking by Rep. Other ways of rendering ITRs non-resolvable are within the ordinary skill in the art.

Further information about AAV viral and vector replication, as well as design of scAAV, can be found in McCarty, D M, et al., Gene Therapy, 10:2112-18 (2003); McCarty, D M, et al., Gene Therapy, 8:1248-54 (2001); McCarty, D M, Molecular Therapy, 16 (10): 1648-56 (2008); U.S. Pat. No. 7,790,154.

In some embodiments, AAV vectors of AAV vectors of the disclosure can contain one or more AAV ITRs originating from different AAV serotypes and variants, have different sequences and lengths, and be positioned at different locations within the genome. The sequence of AAV ITRs for use in the vectors of the disclosure can be wild-type or modified. In other embodiments, AAV ITR sequences can also be included as part of vector sequences in plasmids and other types of vectors, such as baculoviruses, used to introduce the vector sequence into host cells for purposes of vector production.

ITRs from AAV2 are frequently used in producing AAV vectors, but alternative embodiments can include using ITRs from any AAV serotype or variant including, for example, ITRs from AAV1, AAV3, AAV3B, AAV4, AAV5, AAV6, AAV7, AAV8, or AAV9, or any other AAV serotype or variant known or yet to be discovered, so long as such ITRs are functional for vector replication and packaging, and transgene expression. ITRs could also include modifications to the sequence of a wild-type ITR sequence or be fully synthetic. A modified ITR can be at least 90%, 91%, 92%, 93%, 94%, 95%, 96%, 97%, 98%, or 99% identical to a wild-type ITR sequence, such as that from AAV2. In some embodiments, an ITR is modified by adding, deleting and/or changing nucleotides to disrupt a terminal resolution site (trs), and/or a D sequence of an ITR, or to change some other subsequence with an ITR. A vector, in some embodiments, can comprise ITRs from different AAV serotypes or variants.

In some embodiments, the ITRs chosen for use in vector production will be from the same serotype or variant as the capsid. For example, AAV2 ITRs can be used in conjunction with AAV2 capsids. In other embodiments, however, vectors can be pseudotyped or hybrid, meaning that a vector with ITRs from one serotype or variant can be packaged into a capsid from a different serotype or variant. For example, a vector with ITRs from AAV2 could be packaged into a capsid from AAV8 (or any other serotype or variant capsid). This pseudotype is often abbreviated AAV2/8, where the number before the slash indicates the origin of the ITRs, and the number after the slash indicates the origin of the capsid.

According to some embodiments, AAV vectors of the disclosure can contain vectors with different numbers of ITRs. For example, intact AAV viral genomes usually possess two ITRs, one each positioned at the 5′ and 3′ termini respectively, and AAV vectors produced using intact ITRs and packaged with ssDNA genomes can also have two ITRs similarly positioned. As discussed, above, however vectors can be designed in such a way that their genomes include three ITRs, two at the ends of the genome as in virus or conventional vectors, but one additional at or near the middle (intact or mutated), as in self-complementary vectors. It has also been observed, however that with some vectors, particularly if the genome length exceeds the average packaging capacity of a capsid, 5′ ITRs may be truncated or missing entirely due, it is hypothesized, to premature or variable packaging termination. Although defective interfering particles can result, it is also hypothesized that such particles could nevertheless support transgene expression as a result of complementation of otherwise incomplete genomes (Kapranov, P. et al., Hum. Gene Ther., 23:46-55, 2012). Accordingly, in some embodiments AAV vector particles of the disclosure can contain genomes comprising a single functional ITR, such as at the 3′ end or the 5′, and where at the opposite end is a non-functional truncated ITR, or no ITR sequence. Thus, in some embodiments of AAV vectors of AAV vectors of the disclosure, an AAV ITR can be positioned at the 5′ end of the genome, or at the 3′ end of the genome, or at both the 5′ and 3′ ends of the genome, as well as in other positions.

Another source of heterogeneity that can occur in AAV vectors can arise from the differential presence of the flip and flop ITR configurations in any particular genome. Thus, for example, in some embodiments, any particular vector particle in a sample might contain a genome with an ITR at both ends of the genome in the flip configuration, or with an ITR at both ends of the genome in the flop configuration, or with a flip ITR at the 5′ end and a flop ITR at the 3′ end, or a flop ITR at the 5′ end and a flip ITR at the 3′. When combined with the observation that single-stranded DNA genomes can occur as positive or negative stranded, a sample of AAV vectors could comprise eight possible configurations, in similar or potentially different proportions. Unless otherwise specified, genomes of AAV vectors of the disclosure are not limited to any of these configurations; any or all could be packaged by such vectors.

In some embodiments, a AAV vector includes an intact full-length ITR at each of its 5′ and 3′ ends. Thus, for example, if both ITRs are from AAV2, such full-length ITRs would be 145 nucleotides long. In other embodiments, however, one or more of the ITRs may be truncated, missing one or more terminal nucleotides relative to the canonical full-length sequence for that type of ITR. Thus, for example, an ITR may lack at least or about 1, 2, 3, 4, 5, 6, 7, 8, 9, 10, 11, 12, 13, 14, 15, 16, 17, 18, 19, 20 nucleotides, or more, from its terminal end relative to the canonical full-length sequence for that type of ITR. Such truncated ITRs can occur in genomes packaged by AAV vectors of the disclosure, but also in vector sequences used in the production of AAV vectors, such as in a plasmid. For example, it has been observed that truncated ITRs can still function to produce AAV due to capacity for self-repair if sequences missing from one ITR are retained in the other (Wang, X-S. et al., J. Mol. Biol., 250:573-80, 1995; Samulski, R. et al., Cell, 33:135-43, 1983).

In some non-limiting and purely exemplary embodiments, the genomes of AAV vectors of the disclosure can contain one or more AAV ITRs comprising, consisting essentially of, or consisting of the nucleotide sequence of any one or more of SEQ ID NO:5, SEQ ID NO:12, SEQ ID NO:13, SEQ ID NO:20, or SEQ ID NO:21, other ITR sequences being possible, or the complement or reverse complement of any of the foregoing specifically recited sequences.

### Sequence Optimization

In some embodiments, one or more sequences within an AAV vector can be optimized to improve its functional characteristics relative to a starting reference sequence. For example, and without limitation, any protein coding sequence in a vector can be codon-optimized relative to the wild-type sequence, based on the degeneracy of the genetic code and codon usage biases known to exist between different species and between proteins expressed at high or low levels in the same species. Such codon biases can be identified using a codon adaptation index for a particular species, for example. Codon adaptation index (CAI) is explained in more detail in Sharp, P M and Li, W H, Nucleic Acids Res, 15:1281-95 (1987). In some embodiments, coding sequences are human codon-optimized, meaning the coding sequences are optimized based on human codon biases. Codon-optimization can be facilitated using various algorithms known in the art. As is known in the art, different CAI can be constructed based upon which highly expressed genes, such as human genes, are analyzed. An exemplary human CAI is reported in Haas, J, et al., Current Biology, 6 (3): 315-24 (1996). If desired, protein coding sequences can be codon-optimized for species other than human as well.

With the goal of increasing protein expression levels, different codon-optimization strategies have been proposed and implemented. For example, the most frequently used synonymous codon (i.e., one coding for the same amino acid) can be substituted at each position where it does not occur. Alternatively, codon usage can be adjusted over the entire coding sequence so that it is proportional to the natural codon bias distribution of the host organism. In some embodiments, codon replacement is limited to ones that occur relatively rarely in highly expressed proteins in a species, for example, with a frequency of 10% of less, as reflected in a CAI.

In some embodiments, a protein coding sequence to be expressed by a AAV vector of the disclosure can be codon-optimized by substituting at least one rare codon with a more common synonymous codon. In some embodiments, at least 5%, 10%, 20%, 30%, 40%, 50%, 60%, 70%, 80%, 90%, or 95%, and in some embodiments 100% of rare codons in the protein coding sequence are replaced with a more frequently used synonymous codon as reflected in a CAI, such as a human CAI. In some embodiments, a rare codon is one that occurs at a frequency of less than or equal to 10%, 9%, 8%, 7%, 6%, or 5%, as reflected in a CAI, such as a human CAI.

In some embodiments, a protein coding sequence to be expressed by an AAV vector of the disclosure can be codon-optimized by replacing one or more codons with a more frequently used synonymous codon as reflected in a CAI, such as a human CAI, so that the CAI value calculated for the overall coding sequence is increased relative to the starting non-codon optimized sequence, which in some embodiments is the wild-type coding sequence of a protein. Thus, in some embodiments, the CAI value of a starting reference sequence is calculated with reference to a particular CAI reference table and one or more codons are replaced with more frequent synonymous codons so that the overall CAI value of the now codon-optimized coding sequence is increased by at least or about 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.55, 0.60, or 0.70.

As is known in the art, the presence of hypomethylated CpG dinucleotides in nucleic acid can stimulate immune responses that eliminate transduced cells. Depleting CpG dinucleotides in vectors can therefore increase the likelihood that vector transduction will result in long-term gene expression. Wright, J F, Mol Ther, 28 (3): 701-3 (2020). In view of the potentially detrimental effects of CpG dinucleotides, in some embodiments, any sequence within the genome, including for example, enhancers, promoters, introns, open reading frames that encode protein or functional RNA, transcriptional terminators, 5′ and/or 3′ untranslated region (UTR) sequences, ITRs, or any other sequence can be modified to remove one or more CpG dinucleotides, as long as doing so does not unacceptably interfere with or disrupt some desirable function of the modified element. Because the function of certain elements within vectors, such as ITRs, promoters, and enhancers, can be highly dependent on the identity of particular nucleotides in certain positions, there may be more limited opportunities to significantly deplete such elements of CpG dinucleotides. Because AAV vectors of both polarities (e.g., sense and antisense, with respect to the coding sequence of a transgene within the genome) are packaged into capsids in about equal proportions, a strategy of CpG depletion can, in some embodiments, be directed to reducing or eliminating CpG motifs from the nucleotide sequence of vectors of both polarities, not just vectors that contain protein coding sequence in the sense orientation.

In some embodiments, at least 5%, 10%, 20%, 30%, 40%, 50%, 60%, 70%, 80%, 90%, 95%, 96%, 97%, 98%, or 99% of CpG dinucleotides in a coding sequence or the overall vector sequence (with respect to the sense and/or antisense strand) are deleted, or replaced, relative to a reference starting sequence. In other embodiments at least 1, 5, 10, 15, 20, 25, 30, 35, 40, 45, 50, 55, 60, 65, 70, 75, 80, 85, 90, 95, 100, or more CpG dinucleotides, or a range between any of the foregoing values, are deleted, or replaced, relative to a starting reference sequence. In other embodiments, between 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, or 95-100, CpG dinucleotides are deleted, or replaced, relative to a reference starting sequence.

In other embodiments, sequence optimization can increase or decrease the overall GC content relative to a starting reference sequence. Thus, in some embodiments, the overall percentage of G or C nucleotides in a transgene or the genome overall, can be increased, relative to a starting reference sequence, such as a wild-type protein encoding sequence, by at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, or 40 percent, or more. In other embodiments, the overall percentage of G or C nucleotides in a transgene or the genome overall, can be decreased, relative to a starting reference sequence, such as a wild-type protein encoding sequence, by at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, or 40 percent, or more.

As will be appreciated by those of ordinary skill, when optimizing coding sequence, the goal of substituting with more prevalent codons for any particular amino acid in a species (such as human) may be incompatible with other optimization strategies because introduction of more frequently used codons could introduce CpG motifs, or elimination of CpG might require use of rarely occurring codons, or codon optimization might increase or decrease GC content in undesired ways. In these instances, it may be necessary to design and test different optimized coding sequences (encoding the same polypeptide) to identify versions that strike an acceptable balance between the different optimization strategies to achieve improved protein expression.

Transgene and vector sequence can be optimized by changing features in addition to codon bias and CpG content. For example, any of the following features that sometimes occur in sequences and negatively impact transgene expression can be identified (either conceptually, such as by using algorithms, or empirically) and altered or eliminated so as to reduce their effect: cryptic splice sites; premature transcriptional termination signal sequences (e.g., polyA sequences); translational start sites (e.g., IRES) other than for the intended initiator methionine; sequence regions with high GC content; mRNA 5′ end sequences that can form hairpins; and AU rich elements (ARE) in mRNA 3′ untranslated regions that can be bound by destabilizing RNA binding proteins. Other sequence features that can appear in transgenes and vectors that, when altered, can enhance transgene expression will be familiar to those of ordinary skill in the art.

In other embodiments, transgene or vector sequence can be modified to enhance functionality. For example, the first intended start codon in a protein coding sequences may only weakly support translation initiation from that site, in which case, the surrounding sequence can be altered to match the so-called Kozak consensus sequence for translation initiation in eukaryotes. Kozak, M., Gene, 234 (2): 187-208 (1999).

In some embodiments, CpG depletion (partial or complete), or other types of sequence optimization of the transgene coding sequence, such as codon-optimization, can improve protein expression from the transgene compared to the same vector including a non-optimized reference starting sequence, such as a wild-type coding sequence from which the optimized sequence is derived. Thus, for example, the optimized coding sequence of a transgene may express at least 25%, 50%, 75%, 100%, 150%, 200%, 250%, 300%, 350%, 400%, 450%, 500%, or more efficiently compared to a non-optimized reference starting sequence, such as the wild-type version of the coding sequence.

### AAV Capsids

AAV vectors of the disclosure can utilize any AAV capsid, whether naturally occurring, modified, or engineered, including those presently known, or yet to be discovered or developed, which are suitable for transducing cells in a subject to express PGRN protein, or a variant thereof, from a vector transgene.

Choice of which capsid to use in designing and producing an AAV vector (and the corresponding cap gene sequence to be used in its production) can be guided by many considerations and factors. As noted above, by virtue of interacting specifically with certain cell surface receptors, different AAV capsids can have different cell or tissue tropisms, which can be an advantage when it is desired to preferentially transduce certain tissues versus others. For example, to express a transgene product preferentially in brain, such as in a neuron, one might design and produce a vector using a capsid with greater tropism for neurons compared to, for example, muscle or liver. Conversely, to express a transgene product in liver cells, one might design and produce a vector using a capsid with greater tropism for liver cells compared to neurons, muscle, or other tissues.

Other factors may be important as well. It has been reported, for example, that some humans have high neutralizing antibody titers to certain capsids as a result of exposure to naturally occurring AAVs, which can interfere with the ability of AAV vectors with the same or similar capsids to transduce target cells. Thus, in designing a vector for gene therapy, choice of capsid may in some cases be guided by the immunogenicity of the capsid, and/or the seroprevalence of the patients to be treated. Other considerations that may influence capsid choice include manufacturability and stability during storage, with other relevant guiding factors being known in the art.

AAV vectors of the disclosure can use capsids made from capsid proteins from naturally occurring AAVs, as well as modified or engineered capsid proteins. For example, naturally occurring capsid proteins can be modified by inserting or deleting amino acids or peptides, or by introducing amino acid substitutions, in the VP1, VP2, and/or VP3 protein sequence intended to improve capsid function in some way, such as tissue tropism, immunogenicity, stability, or manufacturability. Other examples include novel capsids with improved properties created by swapping amino acids or domains from one known capsid to another (e.g., mosaic or chimeric capsids), or using DNA shuffling and directed evolution methods to discover capsid protein sequences with desired properties.

In some embodiments, AAV vectors of the disclosure can comprise a capsid from known AAV serotypes and variants, as well as non-naturally occurring capsids, including, without limitation AAV1, AAV2, AAV3A, AAV3B, AAV4, AAV5, AAV6, AAV7, AAV8, AAV9, AAVrh10, AAVrh74, AAV-DJ, AAV-PHP.B, Anc80, AAV2.5, and AAV218, with many others being possible. In some embodiments, capsids of AAV vectors of the disclosure include a VP1, a VP2, and/or a VP3 AAV capsid protein that is a variant or derivative of a known VP1, VP2, or VP3 AAV capsid protein. In some embodiments, the amino acid sequence of such variant or derivative AAV capsid protein can be at least or about 70%, 75%, 80%, 85%, 86%, 87%, 88%, 89%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% identical to the amino acid sequence of any known AAV capsid VP1, VP2, or VP3 protein sequence, including, without limitation, the AAV capsid VP1, VP2, or VP3 proteins of AAV1, AAV2, AAV3A, AAV3B, AAV4, AAV5, AAV6, AAV7, AAV8, AAV9, AAVrh10, AAVrh74, AAV-DJ, AAV-PHP.B, Anc80, AAV2.5, and AAV218, or any other suitable AAV capsid, including, for example, the neurotropic capsids discussed below. In some other embodiments, the amino acid sequence of such variant or derivative AAV capsid protein differs (whether due to deletion, insertion, or substitution of amino acids) by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, or more amino acids from a known AAV capsid VP1, VP2, or VP3 protein amino acid sequence including, without limitation, the AAV capsid VP1, VP2, or VP3 proteins of AAV1, AAV2, AAV3A, AAV3B, AAV4, AAV5, AAV6, AAV7, AAV8, AAV9, AAVrh10, AAVrh74, AAV-DJ, AAV-PHP.B, Anc80, AAV2.5, and AAV218, or any other suitable AAV capsid, including, for example, the neurotropic capsids discussed below.

### Neurotropic AAV Capsids

In some embodiments, AAV vectors of the disclosure comprise a neurotropic capsid. A neurotropic capsid is an AAV capsid with tropism for neurons. In some embodiments, such neurons can be neurons in the brain, including neurons in certain regions of the brain, such as, without limitation, the cerebral cortex, including cerebral cortex of the frontal lobe, the temporal lobe, the parietal lobe, or the occipital lobe of the brain, or other regions, such as neurons in the basal ganglia, the thalamus, the hippocampus, the limbic system, the olfactory bulb, the retina, the midbrain, or the brain stem, other brain regions being possible. In other embodiments, such neurons can be neurons in the spinal cord, the peripheral nervous system, or the enteric nervous system. In other embodiments, AAV vectors of the disclosure comprise capsids with tropism for cells in the CNS or outside the CNS other than neurons, non-limiting examples of which include astrocytes, microglia, oligodendrocytes, or ependymal cells, other cell types being possible. In some non-limiting embodiments, AAV vectors of the disclosure comprise capsids that have tropism for neurons such as exist within the human brain. As will be appreciated by those of ordinary skill in the art, neurotropism does not necessarily mean that a capsid is capable of transducing only neurons to the exclusion of non-neuronal cells. Rather, a neurotropic capsid is one that exhibits some greater propensity to transduce neurons as compared to some other cell type, even if that propensity is not absolute, or even if the capsid in question exhibits greater propensity to transduce a non-neuronal cell type compared to neurons. In the latter case, one might refer to such capsid is neurotropic, even if not primarily so.

Examples of neurotropic capsids include, but are not limited to: AAV1; AAV2; AAV4; AAV5; AAV7; AAV8; AAV9; AAV-rh.10; AAVv66 (Hsu, H-L. et al., Nat. Commun., 11:3279, 2020); AAV-DJ (Grimm, D. et al., J. Virol., 82:5887-911, 2008); MNM008, MNM004, 9P31, 9P801, AAV-F, AAV-S, CAP-B10, CAP-B22, PHP.V1, AAV9-retro, T2 3Y+T+dH, AAV8 THR, AAV2.5, AAV-B1, AAV-AS (Bjorklund, T. & Davidsson, M., J. Parkinson's Dis., 11:S209-17, 2021); AAV2 HBKO (Sullivan, J. et al., Gene Ther., 25:205-19, 2018); AAV4.18 (Murlidharan, G. et al., J. Virol., 89:3976-87, 2015; Ojala, D. et al., Mol. Ther., 26:304-19, 2017); AAV2-retro (Tervo, D. et al., Neuron, 92:372-82, 2016); AAV-TT (Tordo, J. et al., Brain, 141:2014-31, 2018); AAV-PHP.B (Deverman, B. et al., Nat. Biotechnol., 34:204-9, 2016); AAV-PHP.S and AAV-PHP.eB (Chan, K. et al., Nat. Neurosci., 20:1172-9, 2017); AAV-CAP-B10 and AAV-CAP-B22 (Goertsen, D. et al., Nat. Neurosci., 25:106-15, 2022); AAV-SCH9 (Ojala, D. et al., Mol. Ther., 26:304-19, 2018); neurotropic capsids (US 2022-0042044 and WO 2021/230987); and many others, including an AAV capsid comprising a VP1 protein comprising or consisting of the amino acid sequence of SEQ ID NO:1, and/or a VP2 protein comprising or consisting of the amino acid sequence of SEQ ID NO:2, and/or a VP3 protein comprising or consisting of the amino acid sequence of SEQ ID NO:3, which may be referred to herein as an AAV-801 capsid.

### AAV Vectors for Expressing Progranulin Protein

According to certain embodiments, AAV vectors of the disclosure comprise an AAV vector comprising a transgene encoding a wild-type progranulin (PGRN) protein, such as human PGRN protein, or a naturally occurring or engineered variant thereof. In some embodiments, the transgene comprises coding sequence for a signal peptide sequence, as well as coding sequence for the mature form of the PGRN protein. In some embodiments, the signal peptide sequence is the same as that in the naturally occurring PGRN protein, but signal peptides from heterologous proteins can be used as well. In some embodiments, the mature human PGRN polypeptide comprises the amino acid sequence of SEQ ID NO:18, and the amino acid sequence of the human PGRN signal peptide comprises SEQ ID NO:17. In some embodiments, the amino acid sequence of wild-type human PGRN protein (including the native signal peptide) comprises the amino acid sequence of SEQ ID NO:16.

In some embodiments, AAV vectors of the disclosure comprise an AAV vector comprising a transgene encoding a variant of the human PGRN protein, such as a variant comprising the insertion, deletion, and/or substitution of at least one amino acid compared to amino acid sequence of wild-type human PGRN protein, e.g., such as that provided by SEQ ID NO: 16. In some embodiments, the transgene encodes a human variant PGRN protein from which at least one, such as 1, 2, 3, 4, 5, 6, 7, 8, 9, 10, 11, 12, 13, 14, 15, 16, 17, 18, 19, or 20, or more amino acids are deleted from the PGRN carboxy-terminus (C-terminus) compared to the amino acid sequence of wild-type human PGRN amino acid sequence, e.g., such as that provided by SEQ ID NO:16. In some embodiments, the transgene encodes a human PGRN variant protein from which the final 3 C-terminal amino acids (QLL) present in wild-type human PGRN protein are deleted (PGRNΔ3). In some embodiments, the amino acid sequence of PGRNΔ3 is identical to that of SEQ ID NO:14. In some embodiments, the C-terminal deletion variants of human PGRN, including the PGRNΔ3 variant, have reduced binding to sortilin (Zheng, Y. et al., PLOS One, 6:1-7, 2011).

In some embodiments, the nucleotide sequence of the transgene encoding human PGRN protein is the wild-type coding sequence, which is identical to the coding sequence as it exists in the exons of the naturally occurring gene encoding human PGRN protein (i.e., GRN) or in the cDNA sequence corresponding to the mRNA transcribed from the GRN gene and encoding human PGRN protein. In some embodiments, the wild-type coding sequence of wild-type human PGRN protein is provided by SEQ ID NO:15, and the wild-type coding sequence of human PGRNΔ3, but for deletion of the codons for the final 3 amino acids present at the C-terminus of wild-type human PGRN, is provided by SEQ ID NO:8. In other embodiments, however, the coding sequence can be optimized, such as by deletion of some or all CpG dinucleotides, while still encoding the same PGRN or PGRNΔ3 protein. In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, or more CpG dinucleotides (or any range encompassing any of the foregoing specifically enumerated values) are removed from SEQ ID NO:15 or SEQ ID NO:8 by replacing codons in CpG dinucleotides appear with synonymous codons lacking CpG dinucleotides. In some embodiments, the nucleotide sequence encoding PGRN or PGRNΔ3 protein is devoid of CpG dinucleotides. In some embodiments, a nucleotide sequence encoding PGRN or PGRNΔ3 protein comprises 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 CpG dinucleotides (or any range encompassing any of the foregoing specifically enumerated values) relative to SEQ ID NO:15 or SEQ ID NO:8, respectively. In some embodiments, the CpG dinucleotides are identified with respect to the order of nucleotides in the sense strand, whereas in other embodiments, the CpG dinucleotides are identified with respect to the order of nucleotides in the antisense strand.

In other embodiments, the nucleotide sequence of the transgene encoding wild-type human PGRN protein is the same as that provided in SEQ NO: 15 (without regard to the stop codon), whereas in other embodiments, the transgene is at least or about 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the nucleotide sequence of SEQ NO: 15, while encoding the identical amino acid sequence as that encoded by SEQ NO: 15 (i.e., SEQ ID NO:16). In other embodiments, the nucleotide sequence of the transgene encoding human PGRNΔ3 protein is the same as that provided in SEQ NO: 8 (without regard to the stop codon), whereas in other embodiments, the transgene is at least or about 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the nucleotide sequence of SEQ NO: 8, while encoding the identical amino acid sequence as that encoded by SEQ NO: 8 (i.e., SEQ ID NO:14).

In some embodiments, vectors of the AAV vectors of the disclosure further comprise at least one AAV inverted terminal repeat (ITR), positioned at the 5′ and/or the 3′ end of the genome. In some embodiments, vectors can further comprise at least a second AAV ITR positioned at the opposite end of the genome from the first AAV ITR. In some embodiments, vectors comprise an AAV ITR positioned at its 5′ terminus. In some embodiments, vectors comprise an AAV ITR positioned at its 3′ terminus. In some embodiments, vectors comprise a first AAV ITR positioned at its 5′ terminus and a second AAV ITR positioned at its 3′ terminus. In some embodiments, vectors comprise a first AAV ITR positioned at its 5′ terminus and a second AAV ITR positioned at its 3′ terminus and a third AAV ITR positioned between said first and second AAV ITRs.

AAV ITRs for use in the vectors of the disclosure can be of any type, such as an AAV2 ITR or a non-AAV2 ITR, and can be full-length or truncated, and can have the same sequence as any known AAV viral ITR as it exists in nature (wild-type sequence) or can be modified. Exemplary non-limiting types of modifications include reducing the number of CpG dinucleotides occurring in the ITR sequence, reducing or eliminating the ability of the ITR sequence to undergo terminal resolution by AAV Rep proteins, such as by mutating, deleting or otherwise inactivating the terminal resolution site (trs), as well as reducing or eliminating the ability of the ITR to support packaging into a capsid, such as by mutating, deleting or otherwise inactivating the D sequence of the ITR sequence. In some embodiments, the vector can further comprise at least a third AAV ITR, such as one positioned between the ends of the genome, such as near or at the center of the vector sequence. In some of these embodiments, the third ITR can be modified, such as by inactivating its terminal resolution site such that the vector, including the transgene, is self-complementary. In some embodiments, vectors comprise an AAV ITR comprising, consisting essentially of, or consisting of the nucleotide sequence of SEQ ID NO:5, SEQ ID NO:12, SEQ ID NO:13, SEQ ID NO:20, or SEQ ID NO:21, or the complement or reverse complement of each of such sequences.

In some embodiments, vectors of the AAV vectors of the disclosure further comprise a transcription control region operably linked with the transgene encoding the PGRN or PGRNΔ3 protein. In some embodiments, the transcription control region can be inducible, constitutively active, or cell-type or tissue-type specific, such as being active mostly or exclusively in neurons or brain (thus, neuron specific or brain tissue specific). In some embodiments, the transcription control region comprises or consists of a promoter and can further comprise at least one enhancer region or element. Any region or element in the transcription control region can be derived from a human gene, or a non-human gene, such as a rat, mouse, bovine, non-human primate, chicken, or viral gene, or other species or type of organism. Regions or elements of the transcription control region can be contiguous with each other or be separated by other functional sequences of the vector. Thus, for example, a promoter region could be proximal and 5′ (upstream) of the transgene, whereas an enhancer region or element could be anywhere else in the vector, such as distally upstream, or elsewhere, such as distally 3′ (downstream) of the transgene. Any region or element of a transcription control region can be cell type or tissue specific, such as brain tissue or neuron (or other brain cell type) specific. Thus, a promoter can be brain tissue or neuron (or other brain cell type) specific, an enhancer region or element can be brain tissue or neuron (or other brain cell type) specific, or both the promoter and enhancer(s), acting alone or in concert, can be brain tissue or neuron (or other brain cell type) specific.

In some embodiments a transcription control region for use in the vectors of the disclosure can contain a promoter sequence derived from the human synapsin 1 (SYN1) gene, where such promoter is the entire human SYN1 gene promoter, or a promoter functional subsequence of such human SYN1 gene's promoter. Thus, for example, the promoter can comprise, consist essentially of, or consist of the nucleotide sequence of SEQ ID NO:6, or a promoter functional subsequence, modification or variant thereof.

In some embodiments, vectors of the AAV vectors of the disclosure further comprise a 5′ untranslated region (UTR) from a gene positioned 3′ of the promoter and 5′ of the transgene encoding PGRN or PGRNΔ3 protein. In some embodiments, the 5′ UTR sequence is the entire 5′ UTR sequence from a gene, or a subsequence thereof. In some embodiments, the 5′ UTR sequence is from the human synapsin 1 gene (SYN1), whereas in other embodiments, the 5′ UTR sequence is from other human synapsin genes, or synapsin genes of other species, or from genes other than synapsin 1. In some embodiments, the 5′ UTR sequence comprises, consists essentially of, or consists of SEQ ID NO:7.

In some embodiments, vectors of the AAV vectors of the disclosure further comprise a transcription termination signal sequence, such as a polyadenylation (poly (A)) signal sequence, such as a poly (A) signal sequence derived from the bovine growth hormone (bGH) gene, or a transcription termination functional subsequence of such bGH gene's poly (A) signal sequence. Thus, for example, the transcription termination signal sequence can comprise, consist essentially of, or consist of the nucleotide sequence of SEQ ID NO:9 or SEQ ID NO: 10, or transcription termination functional subsequences, modifications or variants thereof.

In some embodiments, vectors of the AAV vectors of the disclosure further comprise additional functional sequences, such as an intron, a viral post-transcriptional regulatory element (PRE) sequence, such as a woodchuck hepatitis virus post-transcriptional regulatory element (WPRE), or a Hepatitis B virus posttranscriptional regulatory element (HPRE), any of which can be positioned 3′ of the transgene and 5′ of the transcription termination signal sequence, or elsewhere in the genome. In some embodiments, the PRE can be a WPRE comprising, consisting essentially of, or consisting of the nucleotide sequence of SEQ ID NO:29, or a functional subsequence, modification or variant thereof. Other sequences that may find utility in vectors of the disclosure include, without limitation, a binding site for a microRNA (miRNA), which can be positioned 3′ of the transgene and 5′ of the transcription termination signal sequence, or elsewhere in the genome, and stuffer or filler nucleotide sequences that, while not necessarily intended to directly affect expression of the transgene (although such property could be present) are included so that the overall length of the vector is of a particular size, for example sufficiently long so as to approximate the packaging capacity of a particular AAV capsid, so as to reduce the amount of contaminating non-full-length vector genomic DNA that is packaged in capsids.

In some embodiments, vectors of the AAV vectors of the disclosure further comprise a stuffer or filler sequence positioned 3′ of the poly(A) sisignal sequence and 5′ of an ITR. In some embodiments, the stuffer or filler sequence is derived from an intron of a gene, such as the gene encoding the TATA box binding protein (TBP) of human or another species. In some embodiments, the stuffer or filler sequence is a TBP gene intron, or modification or variant thereof, such as that provided by the nucleotide sequence of SEQ ID NO:11.

In some embodiments, vectors of the AAV vectors of the disclosure comprise a first AAV ITR, a transcription control region, a 5′ UTR sequence, a transgene encoding human PGRN or PGRNΔ3 protein in operable linkage with the transcription control region, a transcription termination signal sequence, a filler sequence, and a second AAV ITR. In related embodiments, these elements can be arranged sequentially in 5′ to 3′ order as would occur in a single-stranded vector in the sense orientation, or in the sense strand of a double-stranded DNA molecule comprising the vector sequence, such as in a plasmid used for producing vectors in host cells. Conversely, these elements can be arranged sequentially in 3′ to 5′ order in a single-stranded vector in the antisense orientation, or in the antisense strand of a double-stranded DNA molecule comprising the vector sequence. Thus, for example, and not by way of limitation, if a vector in the sense orientation comprises a promoter, a transgene, and a poly (A) signal sequence in 5′ to 3′ order, the complementary antisense vector sequence would comprise those same elements in the opposite order starting from its 5′ end, with the understanding that the nucleotide sequence of the antisense stranded genome read in the 5′ to 3′ direction would be the reverse complement of that of the sense stranded genome. In the case of a self-complementary vector (scAAV), the arrangement of elements would occur in both 5′ to 3′ order over about half of its sequence, and then in 3′ to 5′ order in the complementary half.

In some embodiments, vectors of the AAV vectors of the disclosure comprise in 5′ to 3′ order a first AAV ITR from AAV2 at the 5′ terminus of the genome, a transcription control region, a 5′ UTR sequence, a transgene encoding PGRN or PGRNΔ3 protein in operable linkage with the transcription control region, a transcription termination signal sequence, a filler sequence, and a second AAV ITR from AAV2 at the 3′ terminus of the genome. In some embodiments, the transcription control region comprises a promoter, which can be neuron specific, such as a promoter derived from the human synapsin 1 gene that, in some embodiments, can comprise, consist essentially of, or consist of the nucleotide sequence of SEQ ID NO:6. In some embodiments, the 5′ UTR is also derived from the human synapsin 1 gene that, in some embodiments, can comprise, consist essentially of, or consist of the nucleotide sequence of SEQ ID NO:7. In some embodiments, the transcription termination signal sequence is derived from the bovine growth hormone (bGH) gene that, in some embodiments, can comprise, consist essentially of, or consist of the nucleotide sequence of SEQ ID NO:9 or SEQ ID NO:10. In some embodiments, the filler sequence is derived from an intron from a TBP gene that, in some embodiments, can comprise, consist essentially of, or consist of the nucleotide sequence of SEQ ID NO:11.

In any of the foregoing embodiments, the nucleotide sequence of the transgene encoding human PGRN protein can comprise, consist essentially of, or consist of the nucleotide sequence of SEQ ID NO:15, or a nucleotide sequence at least or about 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the nucleotide sequence of SEQ ID NO:15 and encoding the identical amino acid sequence as SEQ ID NO:16. Alternatively, in any of the foregoing embodiments, the nucleotide sequence of the transgene encoding human PGRNΔ3 protein can comprise, consist essentially of, or consist of the nucleotide sequence of SEQ ID NO:8, or a nucleotide sequence at least or about 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the nucleotide sequence of SEQ ID NO:8 and encoding the identical amino acid sequence as SEQ ID NO: 14.

In any of the foregoing embodiments, either or both of the first and second AAV2 ITRs can be full-length or truncated and can be in the flip or flop configuration. In any of the foregoing embodiments, either or both of the first and second AAV2 ITRs can comprise, consist essentially of, or consist of the nucleotide sequence of SEQ ID NO:5, SEQ ID NO:12, SEQ ID NO:13, SEQ ID NO:20, or SEQ ID NO:21, or the complement or reverse complement of each of such sequences.

In some embodiments, vectors of the AAV vectors of the disclosure further comprise a third AAV ITR positioned between the first and second AAV ITRs, such as in the middle of the vector (even if not exactly in the middle) that, in some embodiments, can have a mutated or altered terminal resolution site that does not undergo terminal resolution. In some of these embodiments, the vector can be self-complementary, and can have ranging from about 3000 to 5000, or 4000 to 5000 nucleotides when packaged in a capsid, or length ranging from about 1500 to 2500, or 2000 to 2500 nucleotides when its sequence is contained in a plasmid suitable for use in producing scAAV vectors in host cells.

In any of the foregoing embodiments, the vector can comprise, consist essentially of, or consist of the nucleotide sequence of SEQ ID NO: 19, or the reverse complement thereof. In any of the foregoing embodiments, the vector can be single-stranded, meaning that it is not self-complementary (outside of the ITRs), and can have length ranging from about 3500 to 5000 nucleotides, about 3500 to 4700 nucleotides, about 3800 to 4500 nucleotides, about 3800 to 4300 nucleotides, about 3800 to 4100 nucleotides, about 3800 to 4000 nucleotides, about 3900 to 4000 nucleotides, about 3950 nucleotides, or about 3942 nucleotides. In any of the foregoing embodiments, the vector can be in the sense orientation, or in the antisense orientation.

In any of the foregoing embodiments, the vector can be encapsidated by an AAV capsid, such as a neurotropic AAV capsid, non-limiting examples of which include the capsids AAV1, AAV2, AAV4, AAV5, AAV7, AAV8, AAV9, AAV-rh.10, AAVv66, AAV-DJ, MNM008, MNM004, 9P31, 9P801, AAV-F, AAV-S, CAP-B10, CAP-B22, PHP.V1, AAV9-retro, T2 3Y+T+dH, AAV8 THR, AAV2.5, AAV-B1, AAV-AS, AAV2 HBKO, AAV4.18, AAV2-retro, AAV-TT, AAV-PHP.B, AAV-PHP.S, AAV-PHP.eB, AAV-CAP-B10, AAV-CAP-B22, or AAV-SCH9, or an AAV-801 capsid comprising a VP1 protein comprising or consisting of the amino acid sequence of SEQ ID NO:1, a VP2 protein comprising or consisting of the amino acid sequence of SEQ ID NO:2, and a VP3 protein comprising or consisting of the amino acid sequence of SEQ ID NO:3.

In some embodiments, AAV vectors of the disclosure comprise an AAV-801 capsid encapsidating (packaging) an AAV vector in the sense or antisense orientation, wherein a genome in the sense orientation comprises, in 5′ to 3′ order, a first AAV ITR positioned at the 5′ terminus of the genome, a neuron specific transcription control region, a 5′ UTR sequence, a transgene encoding human PGRNΔ3 protein in operable linkage with the transcription control region, a transcription termination signal sequence, a filler sequence, and a second AAV ITR positioned at the 3′ terminus of the genome, wherein either or both of the AAV ITRs are AAV2 ITRs, wherein the promoter is derived from the human synapsin 1 gene, wherein the 5′ UTR is also derived from the human synapsin 1 gene, wherein the transcription termination signal sequence is a poly (A) signal sequence derived from the bovine growth hormone (bGH) gene, wherein the filler sequence is a modified intron from the human TBP gene, wherein the nucleotide sequence of the promoter comprises, consists essentially of, or consists of the nucleotide sequence of SEQ ID NO:6, wherein the nucleotide sequence of the 5′ UTR comprises, consists essentially of, or consists of the nucleotide sequence of SEQ ID NO: 7, wherein the nucleotide sequence of the transgene encoding human PGRNΔ3 protein comprises, consists essentially of, or consists of the nucleotide sequence of SEQ ID NO:8, or a nucleotide sequence at least 70% identical to the nucleotide sequence of SEQ ID NO:8 and encoding the identical polypeptide as that encoded by SEQ ID NO:8, wherein the nucleotide sequence of the poly(A) sisignal sequence comprises, consists essentially of, or consists of the nucleotide sequence of SEQ ID NO:9 or SEQ ID NO:10, wherein the nucleotide sequence of the filler sequence comprises, consists essentially of, or consists of the nucleotide sequence of SEQ ID NO:11, and wherein a genome in the antisense orientation comprises a nucleotide sequence that is the reverse complement of that of the vector in the sense orientation. In certain related embodiments, the nucleotide sequence of the vector in the sense orientation comprises, consists essentially of, or consists of the nucleotide sequence of SEQ ID NO:19.

### AAV Vector Production

As known in the art, AAV vectors can be produced, including at large scale, in a variety of ways. AAV vectors, for example, can be made in mammalian or insect cells and then purified. The traditional approach, which does not rely on coinfection with a helper virus, involves use of three plasmids as discussed above. One plasmid contains genes for helper virus factors, a second contains the AAV genome sequence in double stranded form, and the third contains AAV rep and cap genes. The rep/cap plasmid often contains a rep gene from AAV2, although this is not a requirement, and the cop gene sequence is chosen based on which AAV capsid protein is desired to constitute the capsid. In practice, the three plasmids are often separately replicated in bacteria, purified, mixed in solution together in predetermined proportions, and then mixed with a transfection agent. The transfection mixture is then used to transfect suitable mammalian host cells (in adherent or suspension cell culture) that are incubated for sufficient time (e.g., 48 to 72 hours, etc.) and under conditions sufficient for the host cells to express the helper factors and the rep and cap genes, and for AAV vector to be replicated from its plasmid template and packaged into capsids. In some embodiments, the host cells are HEK293 cells, which constitutively express AdV helper factors E1A and E1B, such that the helper plasmid only need contain the AdV E2A, E4ORF6, and VA RNA genes. Use of other mammalian host cells that do not produce AdV or other viral helper factor on their own would necessitate use of a helper plasmid containing whichever helper factors are missing or are otherwise required. Although the so-called triple transfection method described above is commonly employed, there is no requirement that the genes for the helper factors, and rep and cap genes, be provided on separate plasmids. In principle all these genes could be housed in one plasmid, for example, in which case two plasmids can be used in the transfection.

Seeking more efficient methods of producing AAV vector at large scale, stable cell lines have been created that contain some but not all the components that would otherwise need to be introduced into cells by transient transfection. Packaging cell lines contain stably integrated AAV rep and cap genes. Production of AAV in packaging cells requires them to be transiently transfected with a plasmid containing an AAV vector and infected with a helper virus. It is also possible to produce AAV vectors in packaging cells without transfection by first infecting them with an AdV (either wild-type or in which the E2b gene is deleted) that supplies AdV E1 gene products, which induce rep and cop expression in the cells, as well as helper factors required for AAV replication, followed by infection with a replication deficient hybrid AdV in which an AAV vector replaces the E1 gene in the genome of the hybrid virus.

In another option, producer cell lines contain stably integrated AAV rep and cap genes, and also an AAV vector. Production of AAV in producer cells requires them to be infected with a helper virus. Packaging and producer cells have been described (Martin, J. et al., Hum. Gene Methods, 24:253-69, 2013; Gao, G. et al., Hum. Gene Ther., 9:2353-62, 1998; Clément, N. & Grieger, J., Mol. Ther. Methods Clin. Dev., 3:16002, 2016). Other cellular systems for producing AAV vectors in mammalian cells, including at commercial scale, are possible.

The baculovirus system has also been employed to produce AAV vectors, in which Sf9 insect cells are infected with recombinant baculovirus vectors that variously contain the AAV rep and cop genes and the AAV genome. The exogenous genes are expressed, followed by genome packaging into vector particles within the cells. In early versions of the system, each component, rep, cap, and genome, were carried by three separate baculoviruses. Later, modifications were made, such as combining rep and cap into a single baculovirus, so that only two types of baculovirus were required, as well as producing Sf9 cell lines containing stably integrated AAV rep and cap genes, which only require infection with a single type of recombinant baculovirus containing an AAV vector (Urabe, M. et al., Hum. Gene Ther., 13:1935-43, 2002; Virag, T. et al., Hum. Gene Ther., 20:807-17, 2009; Smith, R. et al., Mol. Ther., 17:1888-96, 2009; Mietzsch, M. et al., Hum. Gene. Ther., 25:212-22, 2014). Other cellular systems for producing AAV vectors in insect cells, including at commercial scale, are possible.

### Host Cells

As used herein, “host cells” means cells suitable for or adapted to in vitro production of AAV vectors. Host cells are often clonal cell lines capable of dividing for multiple generations before senescence stops growth or may even be immortal. To produce vectors, host cells can be modified, transiently or non-transiently, through the introduction of exogenous genetic information designed to direct biosynthesis in host cells of the various components required for AAV vector assembly, notably the AAV capsid proteins, Rep proteins, helper virus factors, and vectors. For example, host cells can be transfected with exogenously supplied nucleic acid, such as in the form of one or more DNA plasmids, containing nucleotide sequences coding for the required vector components.

Various ways are known in the art for transfecting host cells with nucleic acid. These include, without limitation, mixing nucleic acid with certain compounds that can complex with nucleic acids and then be taken up into the cells, including calcium phosphate or cationic organic compounds, such as DEAE-dextran, polyethylenimine (PEI), polylysine, polyornithine, polybrene, cyclodextrin, cationic lipids, and others known in the art. Transfection can also be performed non-chemically via electroporation and more exotic technologies, such as biolistic particle delivery. As known in the art, transfection can be transient or stable. With transient transfection, the transfected nucleic acid exists in the cell for a limited period of time and, in the case of DNA, does not integrate into the genome. With stable transfection, DNA introduced into the cell can persist for long periods either as an episomal plasmid or integrated into a chromosome. Usually, to produce stably transfected cells, a plasmid containing a selection marker gene, as well as nucleotide sequence coding for one or more of the required vector components, is transfected into the cells that are then grown and maintained under selective pressure, i.e., conditions that kill non-transfected cells or transfected cells from which the exogenous DNA, including its selection marker, are lost for some reason. For example, plasmids can contain an antibiotic resistance gene and transfected cells can be selected for by adding the antibiotic to the media in which the cells are grown. In some embodiments, the nucleotide sequence coding for one or more of the required vector components introduced into stably transfected host cells is under the control of an inducible promoter and is not expressed, or only at a low level, unless an environmental factor, such as a drug, metal ion, or temperature increase, which induces the promoter, is introduced as the cells are grown.

In other embodiments, host cell genomes can be modified in a non-transient and targeted fashion using genetic engineering methods, such as knock-in, or gene editing methods, to direct host cells to produce one or more of the required vector components. In other embodiments, nucleotide sequence coding for one or more of the required vector components can be introduced into host cells for purposes of directing production of AAV vectors via transduction, in which host cells are infected with modified viruses containing such nucleotide sequences. Examples of viral vectors useful for such purposes include adenovirus, retroviruses (including lentiviruses), baculoviruses, vaccinia virus, and herpes simplex virus, with others being possible.

Host cells can be any type of cell known in the art to be useful for the purpose of producing AAV vectors. Host cells are often animal cells, with different types or species being possible, such as insect cells or mammalian cells, including rat, mouse, or human cells, with others being possible. In some embodiments, host cells useful for producing AAV vectors of the disclosure are mammalian host cells, examples of which include Hela cells, Cos cells, HEK293 cells (and variants of HEK293 cells, such as HEK293E, HEK293F, HEK293H, HEK293T or HEK293FT cells), A549 cells, BHK cells, Vero cells, NIH 3T3 cells, HT-1080 cells, Sp2/0 cells, NSO cells, C127 cells, AGE1.HN cells, CAP cells, HKB-11 cells, WI-38 cells, MRC-5 cells, or PER.C6 cells, with many others being possible. In some embodiments, host cells useful for producing AAV vectors of the disclosure are insect host cells, examples of which include Sf9 cells, ExpiSf9, Sf21 cells, S2 cells, D.Mel2 cells, Tn-368 cells, or BTI-Tn-5B1-4 cells, with many others being possible. In some embodiments, host cells, including without limitation HEK293 cells, and its variants, can be adapted to growth in suspension culture.

For purposes of producing AAV vectors, host cells are often grown or maintained in culture under controlled conditions conducive to their growth and vector biosynthesis. For example, host cells can be grown in liquid media of defined chemical composition that provides all the nutrients necessary for cell growth and biosynthesis. Exemplary media includes DMEM, DMEM/F12, MEM, RPMI 1640, for mammalian host cells, and Express Five SFM, Sf-900 II SFM, Sf-900 III, or ExpiSf CD, for certain insect cells. Such media may be supplemented with antibiotics, growth factors or cytokines (produced recombinantly or present in animal serum, such as FBS) known to stimulate growth of the particular type of cells in use, as well as other ingredients that may be required for optimal biosynthesis of AAV vectors, but that would otherwise be in limiting supply. Exemplary supplements include essential amino acids, glutamine, vitamin K, insulin, BSA, or transferrin. In addition to the growth media, other culture conditions may be controlled to optimize growth and/or productivity of the cells, such as pH, temperature and CO2 and oxygen concentration.

Host cells in culture can be grown or maintained in many containers known in the art, such as stirred tank bioreactors, wave bags, spinner flasks, hollow fiber bioreactors, or roller bottle, some of which can be designed and configured for single use or multiple use. Depending on the characteristics of the host cells in question, host cells can be grown in adherent cell culture, where the cells attach to and grow while in contact with a physical substrate, or in suspension cell culture, either where single cells float free in the media that sustains them, or while attached to bead microcarriers, which are suspended in the media. As known in the art, various technologies have been developed and can be used to grow host cells to high cell density, such as perfusion culture, which can increase the overall amount of AAV vector generated per production run.

As known in the art, samples of host cells are often maintained in frozen cell banks, such as master cell banks and working cell banks, which facilitate production of biological products in many batches over time, while ensuring consistent performance by the host cells. Before a campaign to produce an AAV vector, a frozen sample of host cells from a cell bank would typically be thawed, seeded into a small culture volume, and grown to ever higher densities or numbers in cultures of increasing volume. When host cells have reached a desired cell density and/or volume in culture, exogenous genetic material can be introduced, such as by transfection with plasmid DNA or infection or transduction with viral vectors, to cause them to begin producing the AAV vector. Alternatively, if using non-transiently modified host cells in which the nucleotide sequence coding for one or more of the required vector components is under inducible control, the environmental factor necessary to induce expression can be introduced. Host cells can then be grown or maintained in culture for time and under conditions sufficient for them to produce the AAV vectors.

### AAV Vector Purification

After biosynthesis in host cells, AAV vectors can be purified in a variety of ways known in the art. For example, in some embodiments, host cells can be lysed mechanically or chemically, such with detergent, after which host cell DNA and other components are removed, followed by steps such as density gradient centrifugation, or use of one or more chromatographic separation methods, to achieve a highly purified preparation of AAV vectors for use in research or methods of treatment.

Chromatography methods useful in the purification of AAV vectors include, without limitation, size exclusion chromatography (SEC); affinity chromatography, using any affinity ligand attached to the chromatography resin or matrix capable of specific binding to a capsid, such as an antibody, lectin, or glycan; immobilized metal chelate chromatography (IMAC); thiophilic adsorption chromatography; hydrophobic interaction chromatography (HIC); multimodal chromatography (MMC); pseudo-affinity chromatography; and ion exchange chromatography (IEX or IEC), such as anion exchange chromatography (AEX) or cation exchange chromatography (CEX).

In some embodiments, AAV vectors can be purified using antibody-based affinity chromatography in which an antibody, or antibody fragment thereof, is attached to a stationary phase (matrix or resin) loaded into a chromatography column through which a host cell lysate is pumped, followed by washing and eluting of vector that had bound to the antibodies. The antibody bound to the solid phase can be an IgG, or fragment thereof, or a single-chain camelid antibody (such as a heavy chain variable region camelid antibody), other types of antibodies being possible. Non-limiting examples of ligand affinity resins include Sepharose AVB, POROS CaptureSelect AAVX, POROS CaptureSelect AAV8, and POROS CaptureSelect AAV9 (Terova, O. et al., BioPharm Intl. eBook pp. 27-35, 2017; Mietzsch, M. et al., Mol. Ther. Methods Clin. Dev., 19:362-73, 2020; Rieser, R. et al., Pharmaceutics, 13:748, 2021).

In other embodiments, AAV vectors can be purified using ligand chromatography in which the stationary phase has attached to it the same type of ligand that certain AAVs are known to use when binding to cells, such as a glycan, sialic acid (e.g., an O-linked or N-linked sialic acid), galactose, heparin, heparan sulfate, or a proteoglycan, such as a heparan or heparin sulfate proteoglycan (HSPG). For example, an affinity matrix containing sialic acid residues can be used to purify AAV vectors with capsids that specifically bind to sialic acid (e.g., AAV1, AAV4, AAV5, or AAV6); an affinity matrix containing galactose can be used to purify AAV vectors with capsids that specifically bind to galactose (e.g., AAV9); and an affinity matrix containing heparin, heparan, or HSPG can be used to purify AAV vectors with capsids that specifically bind to HSPG (e.g., AAV2, AAV3A, AAV3B, AAV6, or AAV13).

Depending on the physicochemical characteristics of the vector, such as the charge on the capsid, AAV vectors can be further purified by performing anion exchange, cation exchange, or hydrophobic interaction chromatography. Other downstream process steps useful for purifying AAV vectors may be used as well, such as, without limitation, desalting and buffer exchange, ultrafiltration, nanofiltration, diafiltration, and tangential flow filtration (TFF). Use of more than one downstream processing step is possible, and a plurality of downsteam processing steps can be performed in any order according to the knowledge of those ordinarily skilled in the art.

### Methods of Treatment

In addition to AAV vectors for expressing PGRN protein, or variants thereof, such as PGRNΔ3, and compositions comprising such AAV vectors, the disclosure provides methods of treating a subject, such as a human subject, in need of treatment for frontotemporal dementia (FTD), frontotemporal lobar degeneration (FTLD), or PGRN deficiency, by administering to the subject a therapeutically effective amount of an AAV vector of the disclosure, or composition comprising such AAV vectors. Also provided is use of an AAV vector for expressing PGRN protein, or variants thereof, such as PGRNΔ3, in the manufacture of a medicament for use in the methods of treatment disclosed herein. There is additionally provided an AAV vector for expressing PGRN protein, or variants thereof, such as PGRNΔ3, or a pharmaceutical composition containing such AAV vectors, for use in the methods of treatment disclosed herein. In some embodiments, the AAV vector employed in the methods of treatment, or comprised by the medicament or pharmaceutical composition is the vector described herein as AAV801-PGRNΔ3, which contains the vector described herein as clone 249.

In some embodiments, the subject has been diagnosed by the time of treatment with FTD or FTLD (or suspected FTD or FTLD) based on standard diagnostic criteria, such as, without limitation, assessment of neurological or psychiatric symptoms or signs, and/or structural or functional brain imaging using MRI, CT, PET, or other brain imaging methods to identify patterns of brain atrophy, in each case that are characteristic of FTD or FTLD. In some embodiments, the subject has been diagnosed by the time of treatment with behavioral-variant frontotemporal dementia (BV-FTD), or non-fluent variant primary progressive aphasia (NFV-PPA). In some embodiments, the subject has been diagnosed by the time of treatment with atrophy in one or more a brain regions, such as, and without limitation, in the frontal lobe (e.g., orbitofrontal cortex, or anterior cingulate gyrus), the temporal lobe (e.g., anterior temporal lobe, medial temporal lobe, or posterior temporal lobe), or other brain regions, such as the inferior parietal lobe, striatum, or thalamus, or other brain regions.

In some embodiments, diagnosis is confirmed using biochemical tests, for example, by detecting lower than normal levels of progranulin protein (PGRN) in samples of serum or cerebrospinal fluid (CSF) obtained from the subject, and/or, in some other embodiments, identifying heterozygous or homozygous deleterious (complete or partial loss of function) mutation or mutations in the GRN gene encoding PGRN in the subject. In some embodiments, the subject experiences haploinsufficiency with respect to the GRN gene and the amount of PGRN produced from either or both GRN alleles. In some embodiments, the subject is diagnosed with FTLD-TDP type A, FTLD-TDP type B, or FTLD-TDP type C, for example, based on neuropathological analysis of brain tissue.

Treatment of subjects with FTD, FTLD, or PGRN deficiency need not result in a cure to be considered effective, where “cure” is defined as either halting disease progression, or partially or completely restoring the subject's health as it was before the onset or worsening of symptoms, or relative to healthy humans without FTD, FTLD, or PGRN deficiency. Rather, a therapeutically effective amount of an AAV vector of the disclosure (including, without limitation, the vector described herein as AAV801-PGRNΔ3), or a pharmaceutical composition containing such AAV vectors, can be one that serves to at least partially reverse, reduce or ameliorate the extent or severity in a subject of at least one symptom or sign associated with FTD, FTLD or PGRN deficiency; or at least partially reverse, reduce or ameliorate the extent or severity in a subject of at least one disorder or dysfunction of the body, organ, tissue, or cell, caused by FTD, FTLD, or PGRN deficiency; or slow the progression of FTD, FTLD, or other deleterious effects of PGRN deficiency in a subject; or improve the quality of life of subjects with FTD, FTLD, or experiencing a deleterious effect of PGRN deficiency. Examples of symptoms, signs, disorders or dysfunctions associated with FTD, FTLD, or PGRN deficiency include, without limitation, cortical or lobar brain atrophy (for example, affecting the orbitofrontal cortex, medial prefrontal cortex, anterior cingulate gyrus, anterior insular cortex, cingulate cortex, insular cortex, inferior parietal lobe, or the anterior, medial, and posterior regions of the temporal lobe), subcortical brain atrophy (for example, affecting the striatum, thalamus, amygdala, or hippocampus), and behavioral consequences of brain atrophy, including for example, Parkinsonism, corticobasal syndrome (CBS), impaired word finding, apraxia of speech, agrammatism, impaired confrontation naming, impaired single-word comprehension, phonological errors, word repetition errors, sentence repetition errors, sentence comprehension errors, surface dyslexia, delusions, hallucinations, and reduced quality of life (QoL).

In other embodiments, a therapeutically effective amount of an AAV vector of the disclosure (including, without limitation, the vector described herein as AAV801-PGRNΔ3), or a pharmaceutical composition containing such AAV vectors, is one that at least partially corrects or changes the value of a biomarker associated with FTD, FTLD or PGRN deficiency to a value that is more reflective of normal function. Examples of biomarkers associated with FTD, FTLD, or PGRN deficiency include, without limitation, lower than normal amounts of PGRN protein in CSF, serum or plasma, or brain tissue, lower than normal amounts of bis(monoacylglycero)phosphate (BMP) in brain tissue, higher than normal amounts of β-hexosaminidase (HexA) and β-galactosidase (β-Gal) enzyme activity in brain tissue, higher than normal amounts of TDP43 fragmentation in brain tissue, and higher than normal amounts of lipofucin in brain tissue.

In some purely exemplary and non-limiting embodiments, the average or median amount of PGRN protein in plasma of humans with deleterious GRN mutations is about 28% of that in healthy humans, and the average or median amount of PGRN protein in CSF of humans with deleterious GRN mutations is about 39% of that in healthy humans (Meeter, L. et al., Dement. Geriatr. Cogn. Dis. Extra, 6:330-40, 2016). Those of ordinary skill the art will appreciate that the average or median value of PGRN protein concentration in biofluids and tissue samples from humans with GRN mutations or healthy humans can vary depending on the assay and sample population, as well as other variables.

In some embodiments, the methods for treating FTD, FTLD, or PGRN deficiency disclosed herein can be used to treat FTD, FTLD, or PGRN deficiency in a human subject with any type of homozygous or heterozygous deleterious mutation in or affecting the GRN gene. Non-limiting examples of deleterious mutations include deletions, insertions, recombinations, splice site variants, missense, or nonsense mutations in either or both alleles of the GRN gene, mutations affecting transcriptional control regions (e.g., enhancers or promoters) of either or both alleles of the GRN gene, and/or mutations that reduce stability of mRNA expressed from either or both alleles of the GRN gene, or the amount of protein translated from such mRNA transcripts, so long as the mutation(s) results in a reduction in the amount of, or loss of, PGRN protein that is produced. Methods for genotyping a subject as having a deleterious mutation in either or both alleles of the GRN gene, such as by RFLP analysis or gene or genomic sequencing, are familiar to those of ordinary skill in the art, as are methods for detecting and quantifying the amount of PGRN protein in a biofluid or tissue sample from a subject.

Therapeutic efficacy of the methods of treatment disclosed herein can be assessed in individual subjects with FTD, FTLD, or PGRN deficiency by observing or measuring and comparing the severity or magnitude of any symptom, sign, disorder, dysfunction, or biomarker value characteristic of FTD, FTLD, or PGRN deficiency before (baseline) and after treatment. Such comparison can be performed at one or more times after treatment, such as at 0, 1, 2, 3, 6, 9, 12, 15, 18, 21, 24, 27, 30, 33, 36, 39, 42, 45, 48, 60, 72, or 84 months, or some other time after treatment. The data used for comparison from individual subjects can be single data points, or the mean of a plurality of data points if available. In some other embodiments, therapeutic efficacy can be assessed in a population (i.e., two or more) of subjects with FTD, FTLD, or PGRN deficiency serving as their own controls by observing or measuring the severity or magnitude of any symptom, sign, disorder, dysfunction, or biomarker value characteristic of FTD, FTLD, or PGRN deficiency among the individuals within the population before (baseline) and after treatment, and comparing the averaged pre-treatment data with the averaged post-treatment data. Such comparison can be performed at one or more times after treatment, such as at 0, 1, 2, 3, 6, 9, 12, 15, 18, 21, 24, 27, 30, 33, 36, 39, 42, 45, 48, 60, 72, or 84 months, or some other time after treatment. In some other embodiments, studies intended to establish and quantify therapeutic efficacy can be designed to compare treatment effects in a population of subjects treated with AAV vectors of the disclosure (treatment arm) to treatment effects in a population of subjects receiving placebo (control arm). Typically, although not necessarily, subjects within treatment and control arms in the study are matched as to relevant subject characteristics, such as age, sex, and disease severity at time of intervention. In other embodiments, the control population is instead defined or determined statistically from a natural history study in which the disease progression of patients with FTD, FTLD, or PGRN deficiency in the absence of any intervention (except perhaps standard of care) is followed.

In some embodiments, the methods for treating FTD, FTLD, or PGRN deficiency, described herein are effective for treating subjects with FTD, FTLD, or PGRN deficiency of any age including, without limitation, subjects who are at least or about 30, 35, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 years of age or older, or an age range encompassing any of the foregoing specifically enumerated ages.

In some embodiments, at the time of treatment with an AAV vector of the disclosure (including, without limitation, the vector described herein as AAV801-PGRNΔ3), or a pharmaceutical composition containing such AAV vectors, a subject has not exhibited any overt signs or symptoms of FTD, FTLD, or PGRN deficiency but has been diagnosed as otherwise likely to develop such signs or symptoms in the absence of treatment based on genetic testing demonstrating the existence of at least one deleterious mutation in either or both alleles of the subject's GRN gene.

In some embodiments, the methods for treating FTD, FTLD, or PGRN deficiency described herein are effective for treating a subject with FTD, FTLD, or PGRN deficiency for a period after administration of an AAV vector of the disclosure (including, without limitation, the vector described herein as AAV801-PGRNΔ3), or a pharmaceutical composition containing such AAV vectors, during which time such subject does not experience any symptoms or signs of FTD, FTLD, or PGRN deficiency, or does not experience any worsening of symptoms or signs of FTD, FTLD, or PGRN deficiency that may have been present at the time of treatment, or at most experiences minimal worsening of symptoms or signs of FTD, FTLD, or PGRN deficiency that may have been present at the time of treatment such that the subject's overall health, function, quality of life, and/or longevity is not substantially or materially impacted. In some embodiments, this period, referred to herein as the period of therapeutic durability, can be any suitable or desired period of time, including for example and without limitation, at least or about 3, 6, 9, 12, 15, 18, 21, 24, or more months, or at least or about 1, 2, 3, 4, 5, 6, 7, 8, 9, 10, 11, 12, 13, 14, 15, or more years, or at least or about 1, 2, 3, 4, 5, 6, 7, or more decades, or any integer value between, or span of time encompassing any of the foregoing specifically enumerated times or even, in some embodiments, the remainder of the subject's life time after receiving gene therapy as described herein.

In some embodiments, the methods for treating FTD, FTLD, or PGRN deficiency described herein are effective to increase the concentration of PGRN protein, or variant thereof, including PGRNΔ3, in cerebrospinal fluid (CSF) of a human subject in need of treatment for FTD, FTLD, or PGRN deficiency to at least or about 5%, 10%, 15%, 20%, 25%, 30%, 35%, 40%, 45%, 50%, 60%, 70%, 80%, 90%, 100%, or 110% of the average concentration of endogenous PGRN protein in the CSF of healthy humans, for example, about 2.9, 3.0, 3.1, 3.2, 3.3, 3.4, 3.5, 3.6, 3.7, 3.8, 3.9, 4.0, 4.1, 4.2, 4.4, 4.5, 4.6, 4.7, 4.8, 4.9, 5.0, 5.1, 5.2, 5.3, 5.4, 5.5, 5.6, 5.7, 5.8, 5.9, 6.0 ng/ml, or higher, or range encompassing any of the foregoing specifically enumerated values, other values being possible depending on the type of assay used to detect and quantify PGRN protein levels.

In some embodiments, the methods for treating FTD, FTLD, or PGRN deficiency described herein are effective to increase the concentration of PGRN protein, or variant thereof, including PGRNΔ3, in cerebrospinal fluid (CSF) of a human subject in need of treatment for FTD, FTLD, or PGRN deficiency by at least or about 5%, 10%, 15%, 20%, 25%, 30%, 35%, 40%, 45%, 50%, 60%, 70%, 80%, 90%, 100%, 125%, 150%, 175%, 200%, 225%, 250%, 275%, or 300%, or more above the average concentration of endogenous PGRN protein in the CSF of such subject prior to treatment, such as in the 1, 2, 3, 4, 5, or 6 months prior to treatment.

In some embodiments, the methods for treating FTD, FTLD, or PGRN deficiency described herein are effective to increase the concentration of PGRN protein, or variant thereof, including PGRNΔ3, in the serum or plasma of a human subject in need of treatment for FTD, FTLD, or PGRN deficiency to at least or about 5%, 10%, 15%, 20%, 25%, 30%, 35%, 40%, 45%, 50%, 60%, 70%, 80%, 90%, 100%, or 110% of the average concentration of endogenous PGRN protein in the serum or plasma of healthy humans, for example, about 100, 105, 110, 115, 120, 125, 130, 135, 140, 145, 150, 155, 160, 165, 170, 175 ng/ml, or higher, or range encompassing any of the foregoing specifically enumerated values, other values being possible depending on the type of assay used to detect and quantify PGRN protein levels.

In some embodiments, the methods for treating FTD, FTLD, or PGRN deficiency described herein are effective to increase the concentration of PGRN protein, or variant thereof, including PGRNΔ3, in the serum or plasma of a human subject in need of treatment for FTD, FTLD, or PGRN deficiency by at least or about 5%, 10%, 15%, 20%, 25%, 30%, 35%, 40%, 45%, 50%, 60%, 70%, 80%, 90%, 100%, 125%, 150%, 175%, 200%, 225%, 250%, 275%, or 300%, or more above the average concentration of endogenous PGRN protein in the serum or plasma of such subject prior to treatment, such as in the 1, 2, 3, 4, 5, or 6 months prior to treatment.

In some embodiments, the methods for treating FTD, FTLD, or PGRN deficiency described herein are effective to increase the concentration of PGRN protein, or variant thereof, including PGRNΔ3, in the brain or spinal cord of a human subject in need of treatment for FTD, FTLD, or PGRN deficiency to at least or about 5%, 10%, 15%, 20%, 25%, 30%, 35%, 40%, 45%, 50%, 60%, 70%, 80%, 90%, 100%, or 110% of the average concentration of endogenous PGRN protein in the brain or spinal cord of healthy humans.

In some embodiments, the methods for treating FTD, FTLD, or PGRN deficiency described herein are effective to increase the concentration of bis(monoacylglycero)phosphate (BMP) in the brain of a human subject in need of treatment for FTD, FTLD, or PGRN deficiency to at least or about 5%, 10%, 15%, 20%, 25%, 30%, 35%, 40%, 45%, 50%, 60%, 70%, 80%, 90%, 100%, or 110% of the average concentration of endogenous BMP in the brain of healthy humans. In some embodiments, the species of BMP that is elevated is BMP 18:1/18:1, BMP 22:6/22:6, or some other species of BMP.

In some embodiments, the methods for treating FTD, FTLD, or PGRN deficiency described herein are effective to decrease the level of β-hexosaminidase (HexA) enzymatic activity in the brain of a human subject in need of treatment for FTD, FTLD, or PGRN deficiency to at most or less than about 95%, 90%, 85%, 80%, 75%, 70%, 65%, 60%, 55%, 50%, 45%, 40%, 35%, 30%, 25%, 20%, 15%, 10%, or 5%, of the HexA enzymatic activity in brain prior to treatment.

In some embodiments, the methods for treating FTD, FTLD, or PGRN deficiency described herein are effective to decrease the level of β-galactosidase (β-Gal) enzymatic activity in the brain of a human subject in need of treatment for FTD, FTLD, or PGRN deficiency to at most or less than about 95%, 90%, 85%, 80%, 75%, 70%, 65%, 60%, 55%, 50%, 45%, 40%, 35%, 30%, 25%, 20%, 15%, 10%, or 5%, of the β-Gal enzymatic activity in brain prior to treatment.

In some embodiments, the methods for treating FTD, FTLD, or PGRN deficiency described herein are effective to decrease the extent or degree of TDP43 fragmentation in the brain of a human subject in need of treatment for FTD, FTLD, or PGRN deficiency to at most or less than about 95%, 90%, 85%, 80%, 75%, 70%, 65%, 60%, 55%, 50%, 45%, 40%, 35%, 30%, 25%, 20%, 15%, 10%, or 5%, of the extent or degree of TDP43 fragmentation in brain prior to treatment.

In some embodiments, the methods for treating FTD, FTLD, or PGRN deficiency described herein are effective to decrease the amount lipofucin in the brain of a human subject in need of treatment for FTD, FTLD, or PGRN deficiency to at most or less than about 95%, 90%, 85%, 80%, 75%, 70%, 65%, 60%, 55%, 50%, 45%, 40%, 35%, 30%, 25%, 20%, 15%, 10%, or 5%, of the amount of lipofucin in brain prior to treatment.

Concentration of PGRN protein, or variant thereof, including PGRNΔ3, as well as that of BMP or lipofucin in biofluids or tissue samples from subjects undergoing treatment with the AAV vectors of the disclosure, or from healthy or other controls, can be detected and quantified using any method known in the art, such as, and without limitation, ELISA, RIA, or LCMS-MS, or any other method known in the art. Enzymatic activity of HexA or β-Gal in biofluids or tissue samples from subjects undergoing treatment with the AAV vectors of the disclosure, or healthy or other controls, can be detected and quantified using any method known in the art, such as, and without limitation, fluorogenic substrate enzymatic assays, or other methods known in the art. TDP43 fragmentation in tissue samples from subjects undergoing treatment with the AAV vectors of the disclosure, or from healthy or other controls, can be detected and quantified using any method known in the art, such as, and without limitation, semi-quantitative Western blot analysis, or any other method known in the art.

### Methods of Prevention

Among other embodiments, the disclosure provides methods for preventing FTD, FTLD, or PGRN deficiency by administering to a subject, such as a human subject, in need of prevention for FTD, FTLD, or PGRN deficiency a prophylactically effective amount of an AAV vector of the disclosure (including, without limitation, the vector described herein as AAV801-PGRNΔ3), or a pharmaceutical composition containing such AAV vectors. Also provided is use of an AAV vector of the disclosure in the manufacture of a medicament for use in the methods of prophylaxis disclosed herein. In addition, there is provided an AAV vector of the disclosure, or a pharmaceutical composition containing such AAV vectors, for use in the methods of prophylaxis disclosed herein.

In some embodiments, administering a prophylactically effective amount an AAV vector of the disclosure (including, without limitation, the vector described herein as AAV801-PGRNΔ3), or a pharmaceutical composition containing such AAV vectors, to a subject in need of prevention for FTD, FTLD, or PGRN deficiency is effective to prevent initiation or onset in the subject of FTD or FTLD; is effective to prevent initiation or onset in the subject of any deleterious effect of PGRN deficiency; is effective to prevent initiation or onset in the subject of a reduction in the amount of PGRN; is effective to prevent initiation or onset in the subject of at least one symptom or sign associated with FTD, FTLD, or PGRN deficiency; is effective to prevent initiation or onset in the subject of at least one disorder or dysfunction of the body, organ, tissue, or cell, caused by FTD, FTLD, or PGRN deficiency; is effective to prevent initiation or onset in the subject of any reduction in the quality of life caused by FTD, FTLD, or PGRN deficiency that, in each case, would otherwise have occurred in the absence of prophylaxis.

According to some embodiments of the methods of prophylaxis described herein, the subject is a human subject with a homozygous or heterozygous deleterious mutation in the GRN gene, the existence of which is determined by genotyping before the onset of any detectable symptom or sign of FTD or FTLD, or other symptom or sign associated with PGRN deficiency. Methods for genotyping, such as by RFLP analysis or gene or genomic sequencing, are familiar to those of ordinary skill in the art.

### AAV Vector Compositions, Formulations, Methods of Administration, Dosages

In addition to AAV vectors, the present disclosure provides compositions comprising such vectors and as at least one pharmaceutically acceptable excipient, diluent, or carrier. Such vectors may be used, among other things, in the methods of prevention and treatment of FTD, FTLD, or PGRN deficiency also described herein.

Compositions comprising AAV vectors of the disclosure can be provided as aqueous solutions or suspensions, emulsions, and in other forms, such as lyophilized cakes. Vector compositions can be formulated using any suitable diluent and excipients that may be necessary to achieve desired properties, such as pH, ionic strength, tonicity, stability, shelf-life, resistance to freeze-thaw cycles, and ability to be freeze dried, as well as considering the mode of administration. Exemplary diluents and carriers include, without limitation, sterile water for injection, ethanol, and glycerol. Exemplary excipients include, without limitation, salts, buffers, acids, bases, surfactants, saccharides, sugar alcohols, and many others known in the art. Compositions comprising AAV vectors of the disclosure for use in preventing or treating a disease or disorder in a subject, such as FTD, FTLD, or PGRN deficiency, can be packaged in any suitable form, such as vials or pre-filled syringes. In some embodiments, kits are provided with a plurality of vials containing sufficient total amount of vector to achieve a desired total dose to be delivered to a particular subject based on relevant variables, such as such subject's disease severity, body mass, sex, or others.

Compositions comprising AAV vectors of the disclosure can be administered by any suitable route of administration, non-limiting examples of which include systemic administration, administration directly into a tissue or organ, intravenous administration, intraarterial administration, intralymphatic administration, intraperitoneal administration, intramuscular administration, intraparenchymal administration, intrathecal administration, intracerebroventricular administration, or intracisternal magna administration, with others being possible.

Compositions comprising AAV vectors of the disclosure can be administered alone, without any other kinds of therapy, or can be administered simultaneously, contemporaneously, or at any suitable dosing interval with a standard of care treatment, or some other agent, compound, drug, treatment or therapeutic regimen. In some embodiments, compositions comprising AAV vectors of the disclosure can be administered after prophylaxis with immunosuppressive agent, such as a steroid or tacrolimus, or other immunosuppressant drug, or immunosuppressant drugs can be administered afterward to control any humoral and/or cellular immune reaction to the gene therapy.

Vector compositions can contain any suitable amount of an AAV vector calculated to deliver a prophylactically or therapeutically effective amount of such vector to a subject in a volume that is easily handled or administered to the subject, and/or would not be expected to cause any discomfort or undesirable side effects to the subject.

In connection with the methods of prevention and treatment provided by the disclosure, AAV vectors and compositions comprising such vectors can be administered in any suitable dose predicted or determined to be effective to achieve the desired degree of prevention or treatment. In some embodiments, doses of an AAV vector of the disclosure for preventing or treating FTD, FTLD, or PGRN deficiency can be quantified and expressed as vectors (vg) per kilogram of subject body weight, abbreviated “vg/kg.” In some embodiments, exemplary efficacious doses of an AAV vector of the disclosure, including, for example, an AAV vector comprising an AAV801 capsid and a genome comprising the nucleotide sequence of SEQ ID NO:19, or reverse complement thereof, include, without limitation, at least or about 1×10⁹ vg/kg, 1×10¹⁰ vg/kg, 1×10¹¹ vg/kg, 1×10¹² vg/kg, 1×10¹³ vg/kg, 1×10¹⁴ vg/kg, or 1×10¹⁵ vg/kg, or a range of doses between and including any of the foregoing specifically enumerated doses, other doses being possible.

Other objects, features and advantages of the present disclosure will be apparent from the foregoing detailed description. It should be understood, however, that the detailed description and the specific examples that follow, while indicating specific embodiments of the disclosure, are given by way of illustration only, since various changes, modifications and equivalents within the spirit and scope of the disclosure will be apparent from the detailed description and examples to those of ordinary skill in the art and fall within the scope of the appended claims.

Unless otherwise indicated, use of the term “or” in reference to one or more members of a set of embodiments is equivalent in meaning to “and/or,” and does not require that they be mutually exclusive of each other. Unless otherwise indicated, a plurality of expressly recited numeric ranges also describes a range the lower bound of which is derived from the lower or upper bound of any one of the expressly recited ranges, and the upper bound of which is derived from the lower or upper bound of any other of the expressly recited ranges. Thus, for example, the series of expressly recited ranges “10-20, 20-30, 30-40, 40-50, 100-150, 200-250, 275-300,” also describes the ranges 10-50, 50-100, 100-200, and 150-250, among many others. Unless otherwise indicated, use of the term “about” before a series of numerical values or ranges is intended to modify not only the value or range appearing immediately after it but also each and every value or range appearing thereafter in the same series. Thus, for example, the phrase “about 1, 2, or 3,” is equivalent to “about 1, about 2, or about 3.”

All publications and references, including but not limited to articles, abstracts, patents, patent applications (whether published or unpublished), and biological sequences (including, but not limited to those identified by specific database reference numbers) cited herein are hereby incorporated herein by reference in their entirety for all purposes to the same extent as if each individual publication or reference were specifically and individually indicated to be so incorporated by reference. Any patent application to which this application claims priority directly or indirectly is also incorporated herein by reference in its entirety.

Unless otherwise indicated, the examples below describe experiments that were or are performed using standard techniques well known and routine to those of ordinary skill in the art. The examples are illustrative.

### EXAMPLES

The following examples as well as the figures are included to demonstrate preferred embodiments. Those of skill in the art will appreciate that many changes can be made in the specific embodiments that are disclosed and still obtain a like or similar result without departing from the spirit and scope described herein.

### Example 1

### Design and Production of AAV Vectors to Express Progranulin (PGRN)

AAV vectors comprising a transgene for expressing human progranulin lacking the last three carboxy-terminal amino acids (PGRNDel3 or PGRNΔ3) were designed, constructed and produced for testing in vitro and in vivo. In optimization experiments, expression of various transgenes encoding truncated human PGRN protein (otherwise wild-type amino acid sequence) in transfected Neuro-2A cells were compared. Codon-optimized and CpG-depleted transgenes produced less progranulin protein compared to wild-type coding sequence (data not shown) and so the latter was employed in the transgene.

The AAV genome, termed Syn-PGRNΔ3 clone 249, comprises in 5′ to 3′ order, a 5′ ITR from AAV2, a promoter derived from the human synapsin gene, 5′ untranslated region (UTR) from a primate synapsin I gene transcript (NCBI Reference Sequence XM_034950363.1), coding sequence for human progranulin polypeptide lacking the 3 carboxy-terminal amino acids (hGRNA3), a stop codon, a bovine growth hormone (bGH) gene polyadenylation (polyA) signal sequence (i.e., transcription terminator), an intron derived from the human TATA-box binding protein (TBP) gene (NCBI Reference Sequence NG_008165.1), and a 3′ ITR from AAV2. With respect to the vector of clone 249, the complete sequence of which is reported as SEQ. ID NO: 17 (inclusive of both ITRs), the nominal starting and ending nucleotide numbers for each genome component is set forth in the following Table 1. Additional polypeptide and nucleotide sequences relating to the vectors described herein are set forth in the Table of Sequences.

TABLE 1

| | Starting | Ending |
|---|---|---|
| | Nucleotide in | Nucleotide in |
| Genome Component | SEQ ID NO: 17 | SEQ ID NO: 17 |
| 5′ ITR | 1 | 145 |
| hSyn Promoter | 164 | 618 |
| 5′ UTR | 619 | 747 |
| hPGRNΔ3 Coding Sequence | 792 | 2564 |
| (including stop codon) | | |
| bGH polyA | 2579 | 2780 |
| TBP Intron | 2787 | 3792 |
| 3′ ITR | 3798 | 3942 |

To produce vector particles, HEK 293 cells in suspension culture were transfected using the classical triple transfection method. HEK 293 cells were expanded from a working cell bank aliquot through multiple passages, starting from shake flask, through wave bag, to single use bioreactor (SUB) at 250 L scale. Cells were transfected by addition of transfection cocktail containing PEI and three different plasmids: pHelper, to express AdV helper factors; pRepCap, to express the AAV capsid proteins (variously AAV6, AAV9, AAVDJ, AAVPHP.B, or AAV801, depending on the experiment) and AAV Rep proteins; and the transgene plasmid. After transfection for 3.5 hours, transfection was quenched by adding CDM4 media, followed by a 72 hour incubation period to allow AAV vector production by the cells. Vector was harvested by lysing the cells with Triton X-100, adding domiphen bromide to flocculate host cell DNA, filtering the supernatant, and then purifying vector in three stages, including affinity chromatography, anion exchange chromatography, and tangential flow filtration. After harvest and purification, vectors were titered by quantitative PCR, and then tested in vitro and in vivo for potency and toxicity. As described below, vectors were then tested in vitro and in vivo to determine if expression of modified progranulin protein (PGRNΔ3) could improve biomarkers associated with GRN haploinsufficiency, leading to FTD in humans.

### Example 2

### In Vitro Testing of AAV GRN Vectors

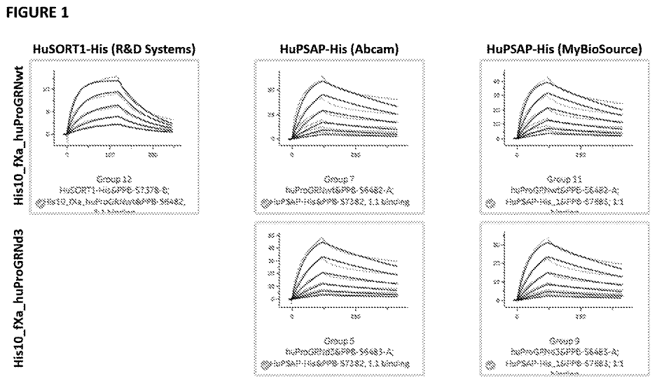

Reports indicate that carboxy-terminal truncations of the human PGRN protein are unable to bind sortilin while retaining the capacity to bind other receptors, such as prosaposin (Zheng, Y. et al., PLOS ONE, 6: e21023, 2011). To confirm this observation, recombinant WT progranulin (PGRNwt) and progranulinA3 (PGRNΔ3) were expressed with a HIS-tag fused to the N-terminal part of the protein, and binding to sortilin and prosaposin assessed in vitro (FIG. 1). Progranulin binding to sortilin requires the terminal three amino acids of WT PGRN, but the binding to prosaposin does not. WT PGRN binds to both human sortilin and prosaposin recombinant proteins. PGRNΔ3 does not bind to sortilin but maintains binding to prosaposin protein as seen with two different sources of prosaposin (Abcam and Mybiosource).

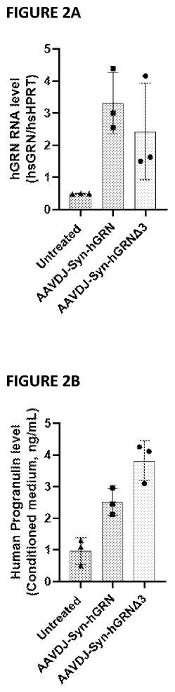

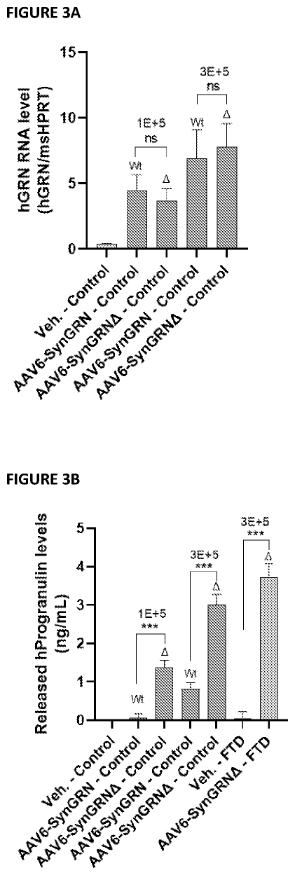

Experiments were designed to determine if human PGRNΔ3 could be produced by different types of neurons in vitro after transduction with AAV vectors for expressing a GRN transgene under the control of a neuronally restricted promoter compared to full-length human PGRN expressed from otherwise identical vectors. Using standard techniques, spinal motor neurons and cortical glutamatergic neurons were differentiated from human induced pluripotent stem cells (IPSCs) and then transduced with AAV vectors for expressing truncated and full-length human PGRN controlled by the SYN promoter. Motor neurons were transduced with vectors using the AAVDJ capsid at a multiplicity of infection (MOI) of 1E5 two weeks after differentiation. Because AAVDJ transduced glutaminergic neurons poorly, AAV vectors using the AAV6 capsid were used in those experiments instead in which two doses of MOI 1E5 and 3E5 were tested. As shown in FIG. 2A, transduction of motor neurons by AAVDJ vectors resulted in comparable levels of mRNA encoding truncated and full-length PGRN. On a protein level, however, motor neurons transduced with AAVDJ vector for expressing PGRNΔ3 resulted in higher levels of PGRN protein in the conditioned media in which the cells were grown compared to the neurons transduced with the vector for expressing the full-length protein, as shown in FIG. 2B. A similar pattern was observed when glutaminergic neurons were transduced with the two types of AAV6 vectors. As shown in FIG. 3A, those neurons produced comparable levels of mRNA encoding PGRNΔ3 and full-length PGRN, which was dose responsive, increasing as the MOI was tripled. Also, similar what was observed with motor neurons, significantly higher levels of progranulin were measured in conditioned media from the cells transduced by AAV6 vectors for expressing PGRNΔ3 than similar vectors for expressing the full-length protein, which was also dose responsive (FIG. 3B). The results from these experiments, showing similar amounts of mRNA but higher amounts of truncated progranulin in conditioned media from cultured neuronal cells compared to full-length was attributed to the inability of truncated protein to bind sortilin, which prevented its being taken back up into the cells after secretion. In another in vitro experiment, it was shown that transduction of cortical glutaminergic neurons differentiated from iPSCs obtained from a human FTD patient were also transduced by AAV6 vectors for expressing PGRNΔ3 and produced detectable levels of progranulin in conditioned media that exceeded the levels produced by similar neurons from a healthy human (FIG. 3B).

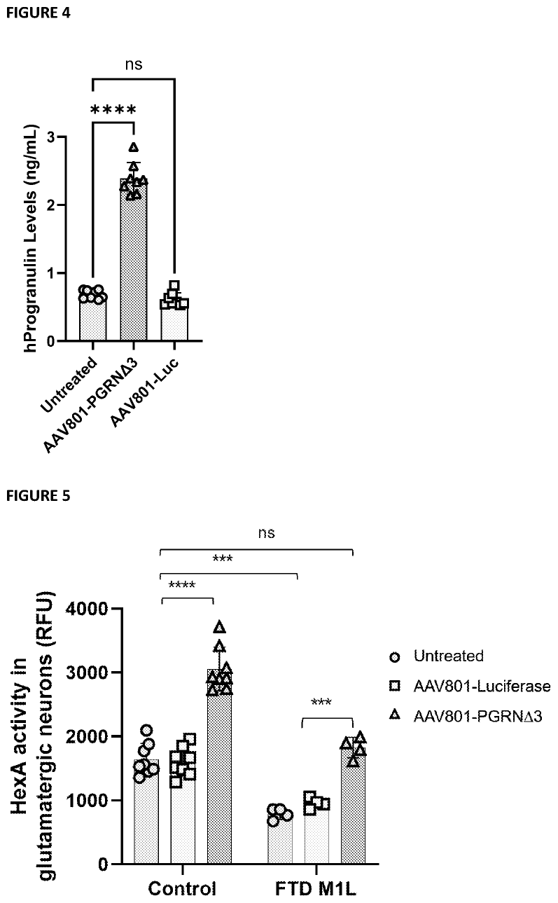

Collectively, the results from the in vitro experiments testing the AAVDJ and AAV6 vectors suggested that transducing neurons in vivo by administering AAV vectors for expressing PGRNΔ3 to the brain could be more effective than previous attempts that relied on vectors for expressing full-length progranulin. These studies were extended by testing whether transducing neurons in vitro with vectors using a capsid capable of crossing the blood brain barrier in NHPs could be similarly effective as the vectors that used AAVDJ and AAV6. Late-stage neuronal progenitor cells from control (WC-30) and FTD (ND50017) iPSC were differentiated into glutamatergic neurons for 14 days in culture prior to addition of AAV801 vectors for expressing human PGRNΔ3 from the clone 249 vector, or control AAV801 vectors designed to produce luciferase (AAV801-Luc). As shown in FIG. 4, mean endogenous human progranulin levels in media from untreated neurons and neurons treated with the control vector AAV801-Luc (MOI 1E6 cells) were 0.69 (SD 0.06) and 0.61 (SD 0.09) ng/ml, respectively, whereas the amount of total detectable hPGRN increased substantially to 2.39 (SD 0.23) ng/ml from the neurons transduced with AAV801-PGRNΔ3 vector (MOI 1E6 cells) (reflecting endogenous full-length PGRN and variant PGRN produced by the transduced cells).

Levels of the lysosomal enzyme β-Hexosaminidase (HexA) are reportedly lower in neurons from FTD patients with GRN haploinsufficiency, and experiments were designed to determine if expressing PGRN in such neurons via vector transduction could increase the levels of this enzyme. The basal activity of β-hexosaminidase in neurons from control iPSC averaged 1641.8 (SD 249.7) relative fluorescent units (RFU), and as expected was lower in differentiated neurons from FTD iPSC, averaging 790.4 (SD 88.8) RFU (FIG. 5). When control and FTD neurons were transduced with AAV801-Luc (MOI 1E6 cells), HexA activity was comparable to untreated control cells of the same type, averaging 1606.5 (SD 225.4) RFU and 956.5 (SD 79.2) in control and FTD neurons, respectively. Transduction of control and FTD neurons with the AAV801-PGRNΔ3 (MOI 1E6 cells), however, increased HexA activity in both about 1.9-fold over the control AAV801-Luc vector, to 3055.1 (SD 344.8) RFU in control neurons and 1825 (SD 160.05) in FTD neurons. HexA activity in FTD neurons after transduction with AAV801-PGRNΔ3 was comparable to that of untreated control neurons (1825 versus 1642 RFU, respectively), suggesting that the AAV801-PGRNΔ3 vector could increase progranulin levels in FTD neurons sufficiently to restore HexA activity to levels comparable to those prevailing in normal neurons.

### Example 3

### In Vivo Testing of AAV Vectors Expressing Full-Length and Truncated PGRN

Having demonstrated that progranulin is present at higher levels in conditioned media of neurons transduced in vitro by AAV vectors for expressing PGRNΔ3 compared to the full-length protein, and that the PGRNΔ3 is bioactive, experiments were designed to test if similar results could be demonstrated in vivo. AAV vectors to express truncated and full-length human PGRN were produced and administered to mice by different routes of administration, after which human progranulin levels expressed from transduced cells were measured.

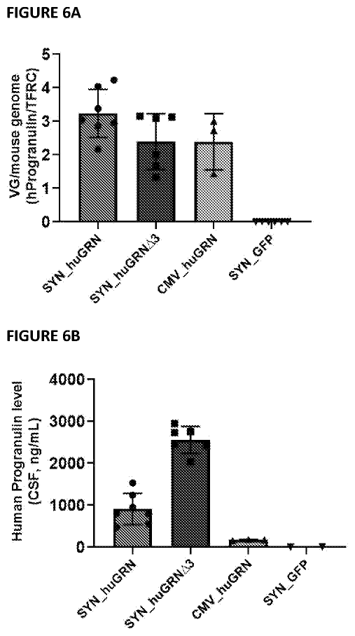

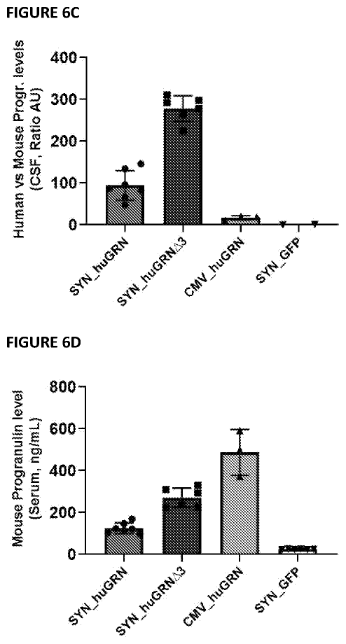

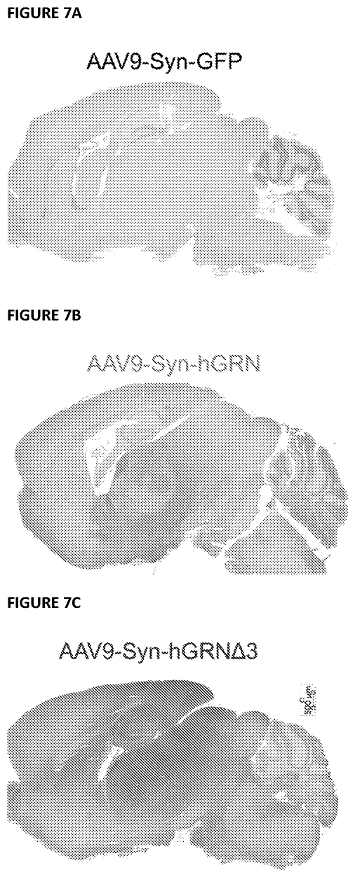

In a first set of experiments, AAV9 vectors to express PGRNΔ3 and full-length PGRN under the control of the neuron-specific synapsin (SYN) promoter were administered bilaterally by the intracerebroventricular route (ICV) into brains (1.5e10 vg/ventricle) of neonatal mice on day P0. Test animals were sacrificed 4 weeks later, and brain tissue, cerebrospinal fluid (CSF), and serum samples taken and analyzed to measure vector transduction and levels and patterns of PGRN expression. As shown in FIG. 6A, both vectors transduced mouse brain tissue at comparable levels, as did a third AAV9 vector designed to express human full-length PGRN under the control of a constitutive CMV promoter. Despite similar levels of transduction, and consistent with the in vitro experiments described above, substantially more human PGRN protein was detected in the CSF of mice transduced with the vector expressing the truncated PGRNΔ3 as compared to full-length (FIG. 6B). A similar result was obtained when expressed protein levels were normalized relative to the amount of endogenous mouse PGRN present in CSF samples from the test animals (FIG. 6C). In contrast to the test animals administered vector containing a transgene encoding full-length human PGRN controlled by a synapsin promoter, much reduced levels of expressed protein were detected in CSF from test animals administered an otherwise similar vector in which the transgene was controlled by a CMV promoter (FIG. 6D). In serum from test animals, however, that pattern reversed, with the vector containing the CMV promoter producing greater amounts of full-length human PGRN protein. Only background levels of PGRN signal were detected in samples from test animals transduced by a negative control vector for expressing a GFP reporter transgene. Brain sections from test animals were also analyzed by immunohistochemistry (IHC) using an antibody specific for human progranulin. No human PGRN signal was detected by IHC in brain sections from animals treated with AAV9 vector expressing GFP (FIG. 7A). By contrast, PGRN staining was detected in brain sections from animals administered AAV9 vectors for expressing full-length and truncated human PGRN (FIG. 7B and FIG. 7C, respectively), but the expression of the truncated PGRNΔ3 protein appeared to be both more intense and diffusely distributed throughout the brain compared to full-length PGRN.

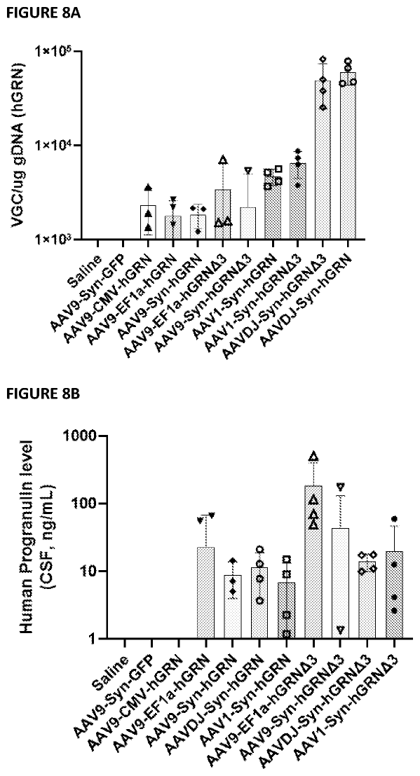

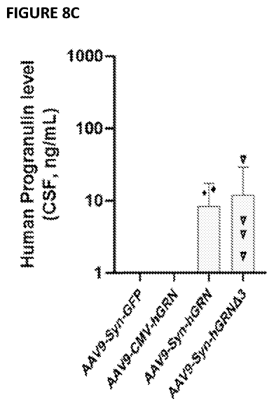

In a second set of experiments, AAV1, AAVDJ and AAV9 vectors to express PGRNΔ3 and full-length PGRN controlled by different promoters were administered unilaterally by the ICV route into brains (5e10 vg/animal) of 6 month old mice. Test animals were sacrificed 3 or 6 months later and levels of progranulin protein secreted into the CSF analyzed. As shown in FIG. 8A, all the vectors transduced brain tissue of the hemisphere contralateral to the injection site, but those employing the AAVDJ capsid were about 10-fold more efficient in doing so compared to AAV1 and AAV9. All vectors for expressing human progranulin, whether truncated or full-length, produced detectable levels of the proteins in CSF of test animals 3 months after treatment. Although the data were variable, there was a trend toward higher expression of PGRNΔ3 compared to full-length PGRN, a pattern that is consistent among the different capsids used to package the expression cassette, and whether the transgene was driven by the neuron-specific synapsin or non-specific EF1a promoter (FIG. 8B). By contrast, another non-specific promoter, CMV, appeared to be inactive in brain even though the AAV9 vector containing it successfully transduced cells. Longer duration of expression was confirmed in a subset of test animals administered AAV9 vectors in which both truncated and full-length progranulin was detected in CSF 6 months after treatment at similar levels measured 3 months earlier (FIG. 8C).

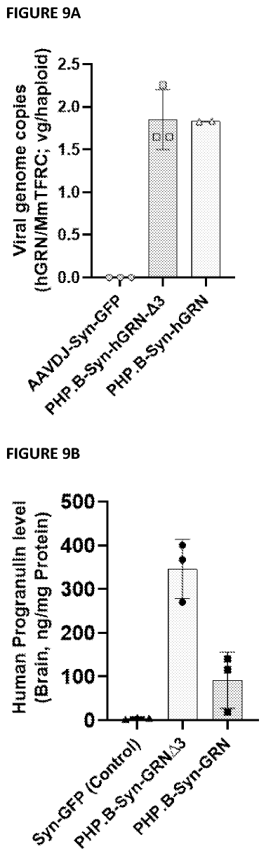

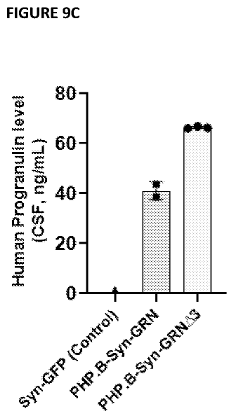

The experiments described above demonstrated that ICV administration of different AAV vectors resulted in expression of truncated and full-length human progranulin detectable in the CSF of treated animals. In a third series of experiments, mice at 6 weeks of age were administered AAV PHP.B vectors (2e13 vg/kg) intravenously (retro-orbitally) to express PGRNΔ3 and full-length PGRN under control of the synapsin promoter. Four weeks after treatment, test animals were sacrificed, and tissues collected for analysis to test if systemically administered vector could result in protein expression in the nervous system. As shown in FIG. 9A, both vectors transduced brain tissue equally and, consistent with the results in which vectors had been administered by the ICV route, truncated progranulin was present at higher levels in both brain tissue (FIG. 9B) and CSF (FIG. 9C) compared to full-length PGRN.

The results from the in vivo experiments described above demonstrated that PGRNΔ3 was present at higher levels than full-length PGRN in the brains of mice of different ages administered AAV vectors using different capsids and by two different routes. Because vectors expressing the two different versions of PGRN protein transduced brain tissue equally efficiently, the most likely explanation for the differences is attributable to the fact that, unlike full-length progranulin, the truncated protein does not bind to sortilin and so remains in the extracellular space after secretion rather than being taken up into cells.

### Example 4

### Testing of AAV Vector-Expressed PGRN Neurotoxicity in Wild-Type Mice

As reported by others, expression of full-length human PGRN protein from AAV9 vectors administered ICV into brains of Grn null mice resulted in selective and dramatic hippocampal toxicity and degeneration affecting neurons and glia, reportedly mediated by a T cell response (Amado D. et al., Mol. Ther., 27:465-78, 2019). Although such unwanted side effects might be controllable in humans, such as administering immunosuppressive agents, experiments were designed to test whether expression of truncated PGRN might reduce or eliminate the neurotoxicity seen with the full-length protein.

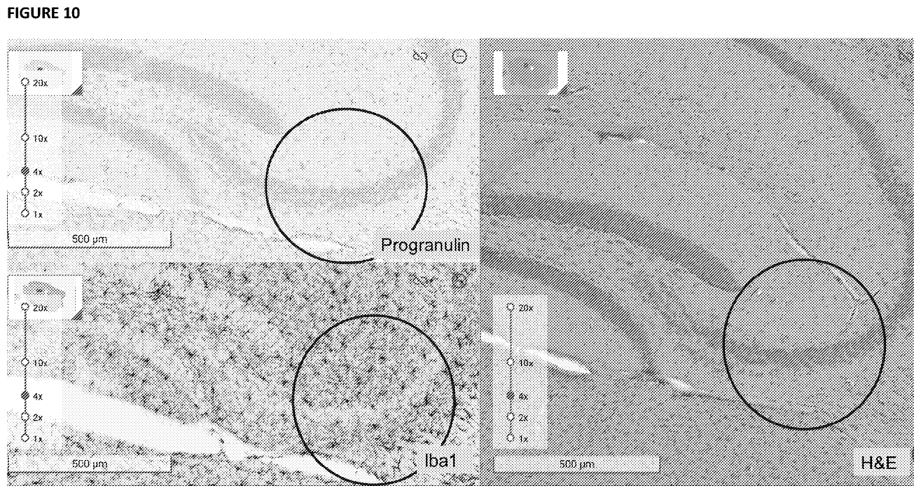

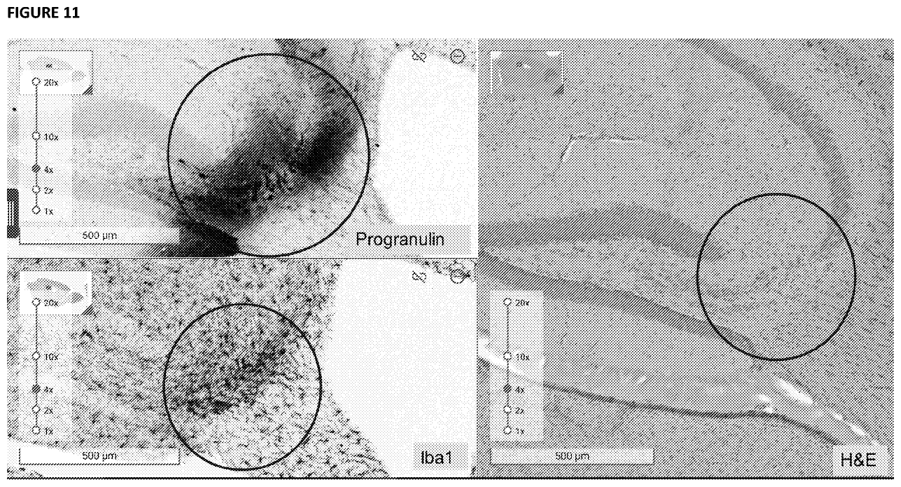

AAV9 vectors for expressing human full-length PGRN and PGRNΔ3 under control of the SYN gene promoter (5e10 vg total each) were administered ICV into brains of 6 month old mice. Three months after treatment, test animals were sacrificed, and brain tissue taken and analyzed. Tissue structure and cellularity of the hippocampal region in coronal brain slices were assessed by H&E staining, and levels of human progranulin protein and the mouse microglial marker Iba-1 were detected by immunohistochemistry (IHC). Prior to sacrifice, no significant differences in body weight or survival were observed between animals treated with PGRN-expressing vectors and negative control animals administered PBS or an AAV9 vector for expressing green fluorescent protein (GFP) (data not shown). In the hippocampal region of test animals administered AAV9 vector expressing GFP, normal levels of mouse Iba-1 but no human PGRN protein were detected by ICH (FIG. 10, left bottom and top micrographs, respectively). Similarly, the hippocampus exhibited normal neuronal cellularity (FIG. 10, right micrograph). In test animals that received AAV9 vectors for expressing human full-length PGRN, progranulin protein was readily detectable in the CA3 region of the hippocampus (FIG. 11, left top micrograph). Unlike the negative control animals, however, the same region exhibited a decrease in cellularity (FIG. 11, right micrograph), as well as increased signal intensity of the microglial marker Iba-1 (FIG. 11, left bottom micrograph), which colocalized with PGRN expression. Together, these results suggested that expression of full-length human PGRN protein was toxic to neurons in the hippocampus and stimulated a robust microglial response, similar to previous reports. Similar results were observed when full-length human PGRN protein was expressed from a vector using the AAVDJ capsid (results now shown), confirming that the toxicity was most likely attributable to the expressed protein and not the vector capsid.

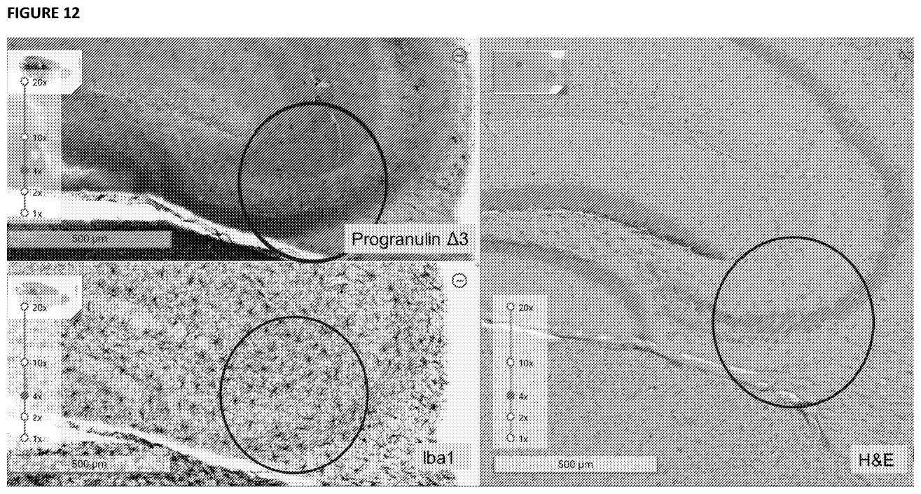

Test animals that received AAV9 vectors for expressing human PGRNΔ3 also had detectable levels of protein in the hippocampus, but its expression pattern was more diffuse compared to the full-length version of the same protein (FIG. 12, left top micrograph). Surprisingly, and by stark contrast, the hippocampal CA3 region from these same test animals had normal appearing neuronal cellularity (FIG. 12, right micrograph), and normal to slightly increased levels of Iba-1 protein (FIG. 12, left bottom micrograph), suggesting no significant microglial activation. Importantly, these results demonstrated that AAV vector mediated expression of the truncated PGRNΔ3 protein in wildtype mouse brain did not cause hippocampal neuronal toxicity, as had been seen with the full-length PGRN, suggesting that expression of the truncated version of the protein via gene therapy may be safer.

### Example 5

### Testing of AAV GRN Vectors in a Mouse GRN Knock-Out Model

A potential advantage of the AAV801 capsid over others is that it has been demonstrated to cross the blood brain barrier (BBB) of non-human primates. AAV801 does not do so in mice, however, which limits the ability to test vectors in that capsid in mouse models of human disease. To avoid this restriction, experiments were designed in which the 249 clone vector was packaged in surrogate AAV9 (AAV9-PGRNΔ3) and AAVPHP.B (AAVPHP.B-PGRNΔ3) capsids for testing in a mouse model for FTD in which the endogenous murine Grn was knocked out (KO). As described further below, Grn null mice demonstrated reduction in two different BMP species (18:1/18:1, 22:6/22:6), increased HexA, increased β-gal activity, and increased TDP43 fragmentation, as well as accumulation of lipofuscin in multiple brain regions, all of which were reversed after treatment with vectors expressing PGRNΔ3.

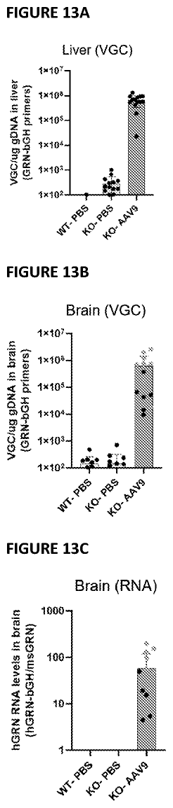

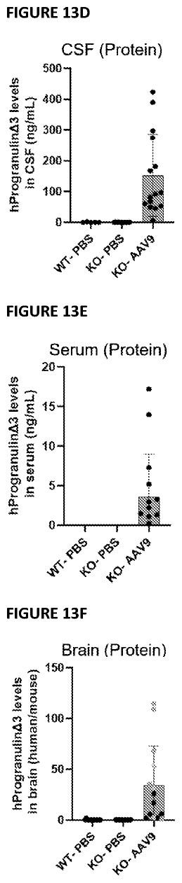

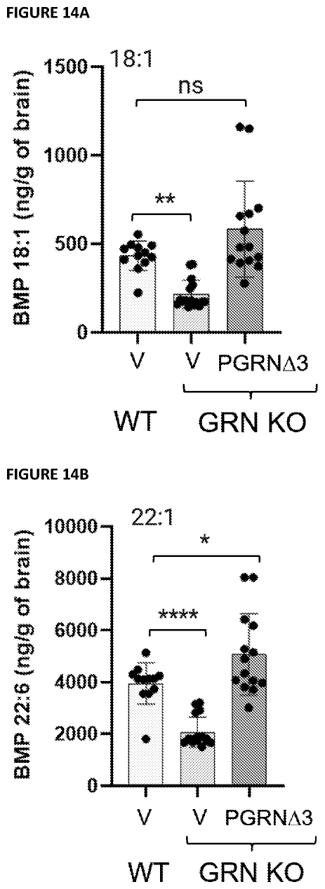

Grn^{-/-} KO mice were injected unilaterally by the intracerebroventricular route (ICV) with AAV9-PGRNΔ3 at 1e11 vg, and aged matched Grn^{-/-} KO and WT mice were injected with PBS as controls. All test animals survived until necropsy and no significant differences on the bodyweight were noted after treatment. Tissues (brain and liver) and biofluids (CSF and serum) were harvested at approximately 2 months post-injection for analysis. One hemisphere of the brain was taken for biochemistry and the other half for immunostaining. Eight right and six left hemispheres were taken from mice injected ICV in the right lateral ventricle to compare distribution from injected and non-injected site of the brain (right hemispheres are depicted in pink dots in FIGS. 13B, 13C, 13E and 13G). Vector genome copies (VGC) in the liver and brain (left or right hemisphere) of AAV9-PGRNΔ3 injected mice averaged 6.64e5 and 6.23e5 vg/μg gDNA, respectively (FIGS. 13A and 13B). The average VGC in the right hemisphere was 1.02e6 vg/μg gDNA and 9.38e4 vg/μg gDNA in the left hemisphere, indicating higher distribution in the injected site versus non-injected site (FIG. 13B). VGC in the liver were also high, likely due to leakage into the blood while test animals were undergoing the ICV injection.

PGRNΔ3 transgene mRNA was also detectable in the brain, averaging 57-fold higher than endogenous murine progranulin mRNA transcripts overall, but was relatively higher in the right hemisphere (i.e., the side of injection), at 77-fold, and relatively lower in left hemisphere, at 31-fold (FIG. 13C). Human progranulin (PGRNΔ3) protein levels averaged 156 ng/ml in CSF, whereas the concentration of endogenous murine progranulin was approximately 2 ng/ml (FIG. 13D). PGRNΔ3 protein levels were low (3.8 ng/ml) in serum, likely due to the use of the synapsin promoter, which was expected to restrict transgene expression to neurons (FIG. 13E). PGRNΔ3 was not detected in the liver (data not shown). In the brain, PGRNΔ3 protein levels averaged 34-fold higher than endogenous murine progranulin, and like mRNA was higher in the right hemisphere, at 51-fold, compared to the left hemisphere, at 10-fold (FIG. 13F).

Grn^{-/-} KO mouse brains exhibit marked deficiency in 18:1/18:1 and 22:6/22:6 bis(monoacylglycero)phosphate (BMP; also known as lysobisphosphatidic acid) compared to age matched WT mice (FIGS. 14A and 14B). BMP is an endolysosomal phospholipid identified as interacting with progranulin in a pH-dependent manner, as well as being a redox-sensitive enhancer of lysosomal proteolysis and lipolysis (Logan, T., et al., Cell, 184:4651-68, 2021). BMP deficiency is reflective of lysosomal defects in the progranulin deficient condition. Tissues from test animals were tested to determine if vector treatment affected BMP levels. In brain tissue from control WT mice, BMP 18:1/18:1 levels averaged 433 ng/g and as expected, was lower in brain tissue from Grn^{-/-} KO mice treated with vehicle, averaging 215 ng/g (FIG. 14A). When Grn^{-/-} KO mice were treated with the AAV9-PGRNΔ3 vector, however, BMP levels in brain increased to an average 583 ng/g (FIG. 11A). Similarly, in WT mice, the concentration in brain of BMP 22:6/22:6 averaged 3947 ng/g and 2074 ng/g in brain of Grn^{-/-} KO mice treated with vehicle, which increased to 5074 ng/g in brain tissue from KO mice treated with AAV9-PGRNΔ3 (FIG. 14B).

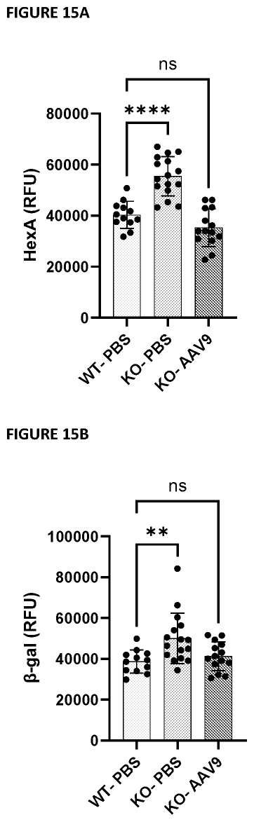

Grn^{-/-} KO mouse brains exhibit increased levels of two lysosomal enzymes, β-hexosaminidase (HexA) and β-galactosidase (β-Gal), compared to age matched WT control mice (FIGS. 15A and 158). Tissues from test animals were tested to determine if vector treatment affected BMP levels. In brain tissue from control WT mice, HexA activity averaged 40309 RFU (SD 5296) and β-Gal activity averaged 38700 RFU (SD 5624), whereas in brains of Gr^{-/-} KO mice treated with vehicle, HexA activity averaged 55430 RFU (7681 SD) and β-Gal activity averaged 50078 RFU (12403 SD). When Grn^{-/-} KO mice were treated with the AAV9-PGRNΔ3 vector, however, both HexA and β-Gal enzyme activity fell to control levels. In vector treated test animals, HexA activity fell to an average of 35300 RFU (SD 7371), similar to WT control level of 40309 RFU (SD 5296), and β-Gal activity fell to an average of 41299 RFU (SD 7095), similar to WT control level of 38700 RFU (SD 5624) (FIGS. 15A and 15B).

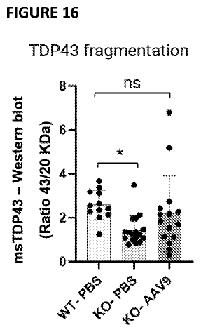

A marker in post-mortem brain of FTD caused by GRN haploinsufficiency is aggregation of TDP43 fragments, and experiments were designed to test for the presence of such fragments in Grn^{-/-} KO mice and whether vector treatment could affect TDP43 fragmentation. Brain lysates from control WT mice, control Grn^{-/-} KO mice treated with vehicle, and Grn^{-/-} KO mice treated with AAV9-PGRNΔ3 vector were prepared and analyzed by Western blot analysis using antibody binding both full-length TDP43 and a TDP43 fragment, migrating at 43 kDa and 20 kDa, respectively. The staining intensity of both bands was quantified, and the ratio of staining intensity of the 43 kDa band to the 20 kDa band calculated, lower ratios indicating relatively more fragmentation. As shown in FIG. 16, Western blot analysis demonstrated that Grn^{-/-} KO mice treated with PBS had a lower 43 kDa band to the 20 kDa band staining ratio compared to age matched WT controls, indicating that the Grn^{-/-} KO mice experienced a greater degree of TDP43 fragmentation in the absence of progranulin expression. When Grn^{-/-} KO mice were treated with AAV9-PGRNΔ3 vector, however, the staining ratio increased and was comparable to that observed in WT controls, suggesting that vector treatment and restoration of PGRN expression reduced TDP43 fragmentation.

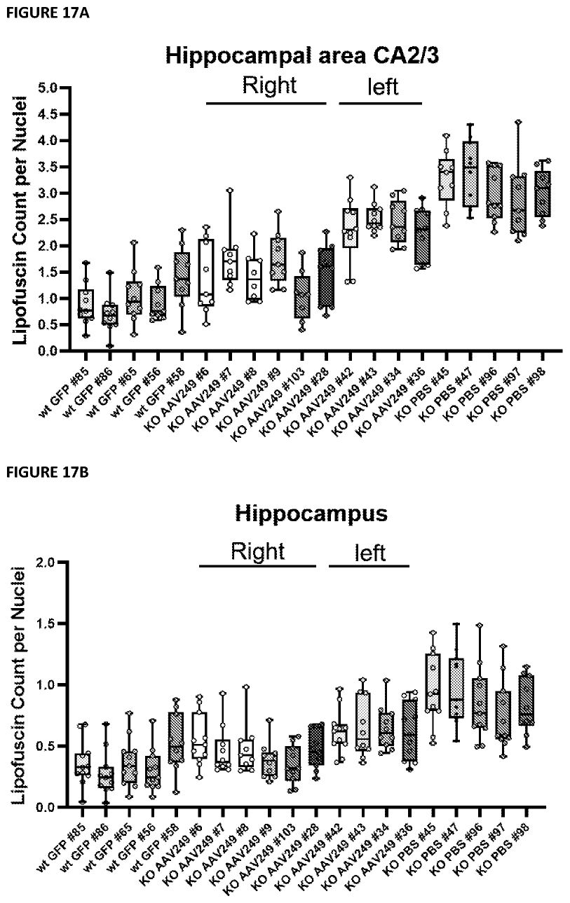

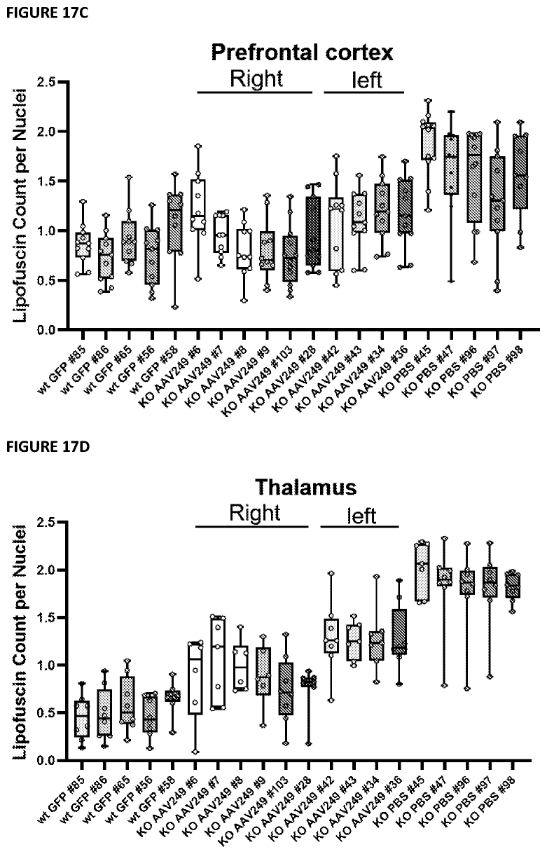

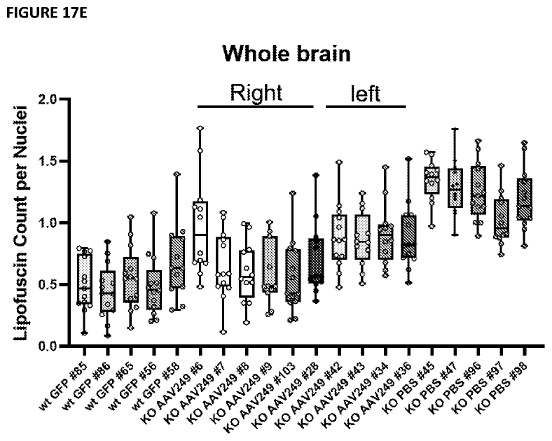

Another biomarker of FTD in the Grn^{-/-} KO mice is accelerated lipofuscinosis characterized by excessive accumulation of lipofuscin in all brain areas as visualized with an auto-fluorescent stain in comparison to WT mice. Lipofuscin staining intensity was quantified in WT mice and treated and control Grn^{-/-}KO mice to ascertain whether this biomarker would also respond to treatment with AAV9-PGRNΔ3 vector. Representative images of lipofuscin staining (shown as red) in hippocampus and thalamus from Grn^{-/-} KO mice treated with vector and vehicle are provided in FIG. 17A. Results from this analysis are provided in FIGS. 17C to 17F, which report lipofuscin levels in different brain areas across one hemisphere moving laterally to distally. Lipofuscin levels were low in WT mice and higher in Grn^{-/-} KO mice administered PBS vehicle or the AAV9-Luc negative control vector. In contrast to control Grn^{-/-} KO mice, when the mice were treated with AAV9-PGRNΔ3 by administering vector into the right ventricle by ICV, reduced lipofuscin accumulation was observed in all treated test animals with a trend toward levels in WT mice, as measured in brain as a whole (FIG. 17B), hippocampal area CA2/3 (FIG. 17C), the entire hippocampus (FIG. 17D), the prefrontal cortex (FIG. 17E) and the thalamus (FIG. 17F). The data also reflect a greater effect of vector treatment reducing lipofuscin levels in the right hemisphere, the side of the brain into which vector was administered, compared to the contralateral left hemisphere.

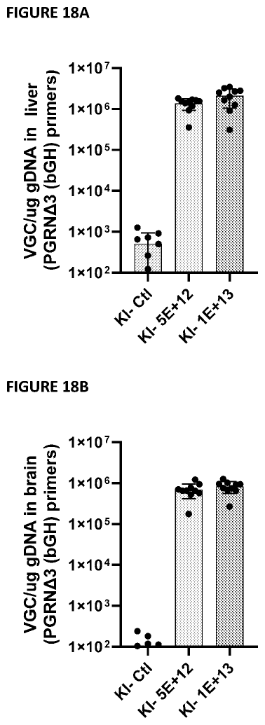

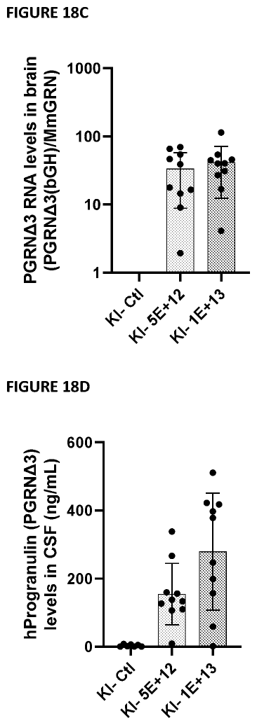

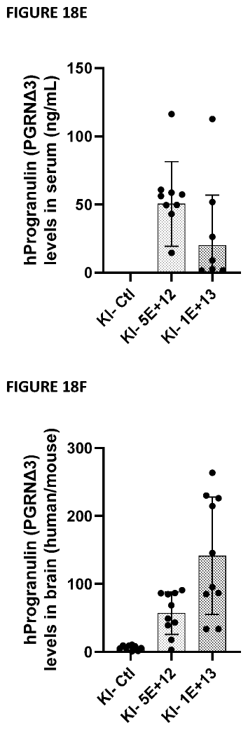

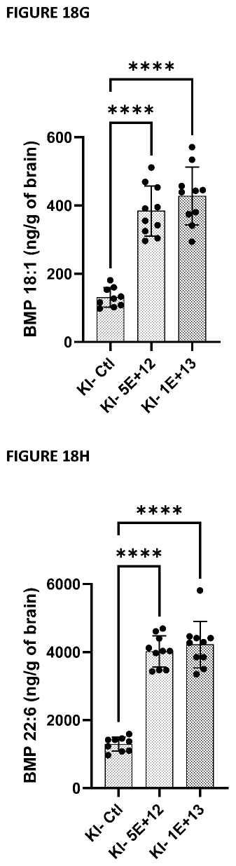

The results described above suggest that administering a vector designed to express PGRNΔ3 via an ICV route is effective to correct biomarkers associated with FTD in humans. Experiments were designed to test whether intravenous administration of vectors capable of crossing the BBB in mice could achieve similar results. The same vector packaged into AAV801 capsids and tested as described above, was packaged into AAVPHP.B capsids that, unlike AAV801, are capable of crossing the BBB in mice (although not in NHPs). AAVPHP.B-PGRNΔ3 vectors were administered intravenously (IV) to Grn null knock-in (KI) mice at doses of 5e12 and 1e13 vg/kg. Both liver and brain exhibited high levels of transduction as detected by quantifying the number of vectors per microgram of genomic DNA from those tissues (FIG. 18A and FIG. 18B, respectively) (in brain, 6.9e5 vg/μg and 8.2e5 vg/μg gDNA at the low and high dose, respectively). In brain, transduction led to high levels of transgene mRNA and PGRNΔ3 protein expression. At the lower vector dose, mRNA expressed from the GRN transgene was about 32-fold greater than the mRNA produced by the endogenous murine Grn gene (FIG. 18C), and PGRNΔ3 protein levels were about 57-fold higher than endogenous murine PGRN protein levels (FIG. 18F). PGRNΔ3 protein levels were also elevated in cerebrospinal fluid (CSF) (FIG. 18D). RNA and protein levels were approximately 2-fold higher in the mice receiving the higher vector dose, indicating dose responsiveness in treatment effect. PGRNΔ3 protein levels were lower in serum FIG. 18E), likely due to use of the synapsin promoter, providing restricted expression of the transgene in neurons.

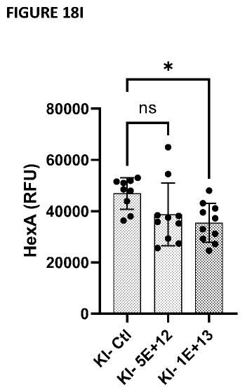

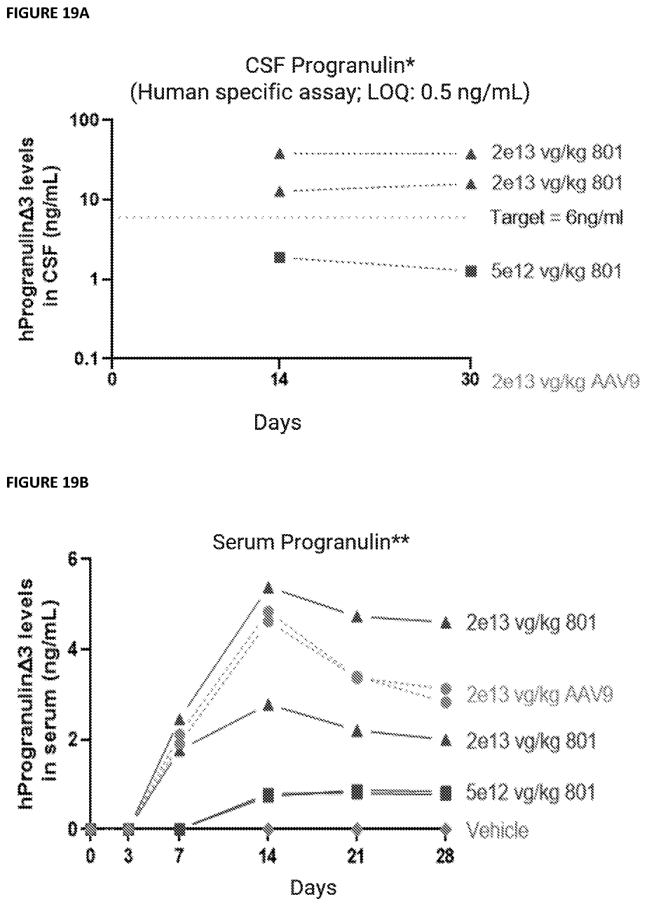

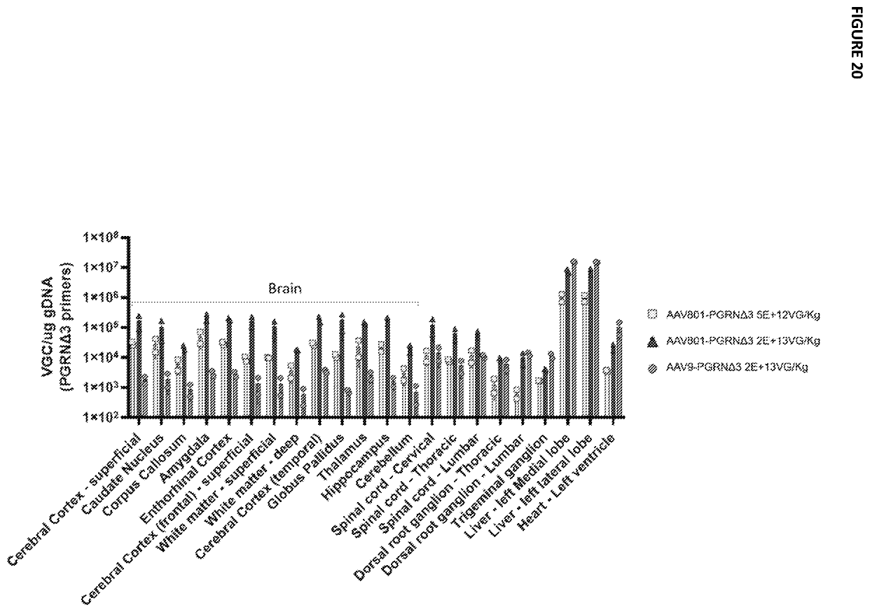

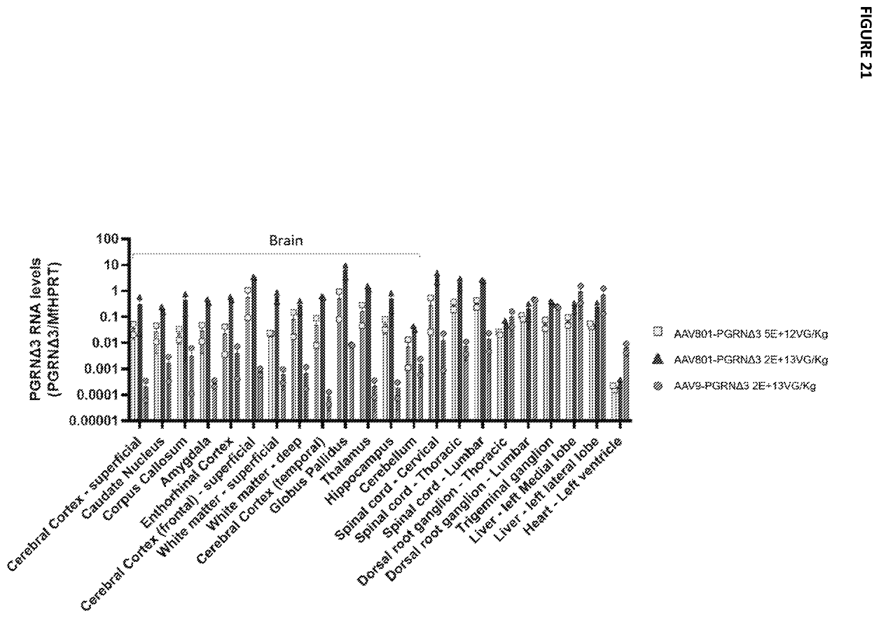

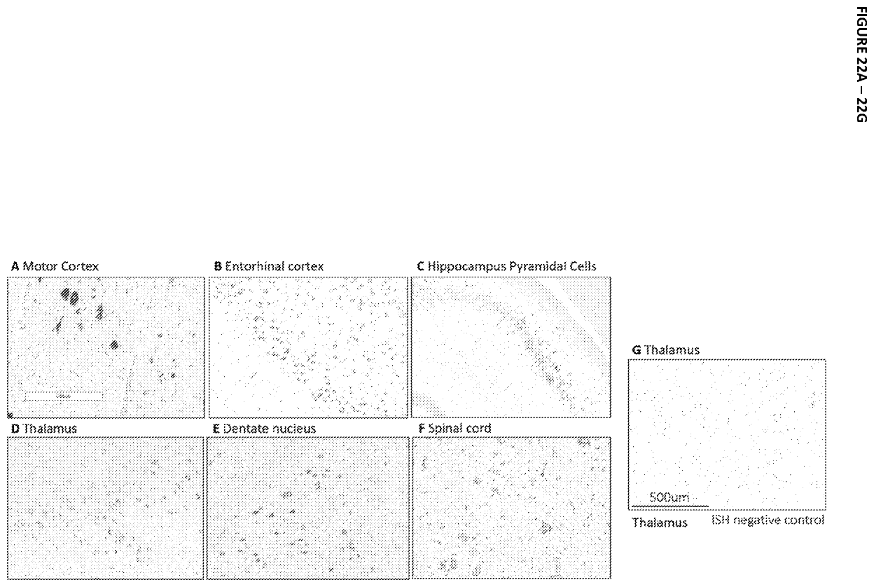

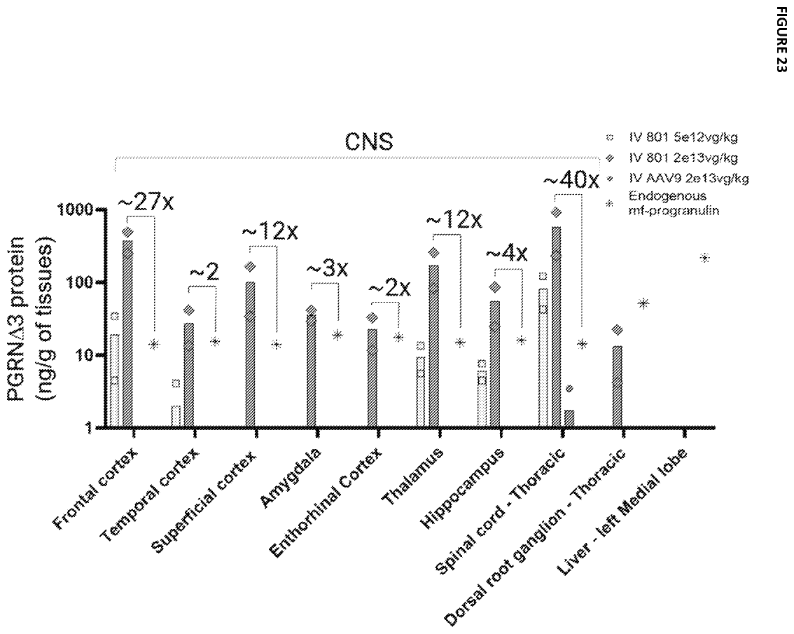

At both doses, brain tissue from all Grn null KI mice treated with the AAVPHP.B-PGRNΔ3 vector exhibited an increase in levels of BMP 18:1/18:1 (mean of 384.1 ng/g at the 5e12 vg/kg dose) and BMP 22:6/22:6 (mean of 427.8 ng/g at the 5e12 vg/kg dose) compared to WT controls (FIGS. 18G and 18H, respectively), whereas HexA enzyme activity was reduced (FIG. 181). Collectively, these results suggest that IV administration of a transgene expressing PGRNΔ3 protein by an AAV capsid capable of crossing the blood brain barrier can transduce brain cells, and express PGRNΔ3 protein at sufficient levels to correct biomarkers associated with FTD in humans.

### Example 6

### Testing of AAV GRN Vectors in Non-Human Primates

A study comparing the biodistribution in cynomolgus monkeys of AAV vectors containing the 249 clone vector packaged in AAV801 and AAV9 capsids was undertaken.

Cynomolgus monkeys were intravenously administered AAV801-PGRNΔ3 and AAV9-PGRNΔ3 vectors, each containing the 249 clone vector to express hPGRNΔ3, at two doses, 5e12 vg/kg and 2e13 vg/kg. Progranulin protein concentration was measured in serum and CSF samples taken just before and up to 28 days after dosing, as were samples of brain and other tissues that were tested to quantify vector copy (VGC) number, and transgene RNA and protein expression. PGRNΔ3 levels in CSF and serum were detected using a human ligand binding assay (LBA) that does not detect the endogenous macaque progranulin.

PGRNΔ3 protein was detected in CSF of test animals treated with AAV801-PGRNΔ3 on day 14 and day 28 (FIG. 19A). The high dose (2e13 vg/kg) produced PGRNΔ3 protein levels of about 50 ng/ml and 20 ng/ml in two test animals, both higher than the average 6 ng/ml progranulin naturally occurring in humans. Lower but still detectable levels of PGRNΔ3 protein was detected in one of two test animals receiving the lower 5e12 vg/kg dose. By contrast, no detectable PGRNΔ3 protein was expressed in the two test animals that received AAV9-PGRNΔ3 at a dose of 2e13 vg/kg. These results suggest that the AAV801 capsid is much more efficient at reaching the brain through the BBB to transduce neurons there compared AAV9. Serum concentration of PGRNΔ3 protein in test animals treated with both AAV801-PGRNΔ3 and AAV9-PGRNΔ3 vectors was comparable (FIG. 19B), and substantially lower than the approximately 200 ng/ml naturally occurring in human serum. Protein was first detected on day 7 and rose to a peak of about 5 ng/ml on day 14 in test animals receiving the high vector dose (2e13 vg/kg) delivered by either the AAV801 or AAV9 capsid. PGRNΔ3 protein levels were below 2 ng/ml in the test animals administered the low dose (5e12 vg/kg) of the AAV801-PGRNΔ3 vector. The low levels of PGRNΔ3 protein in serum may be attributable to use of a neuronally restricted promoter to drive expression of the transgene in transduced cells.

Brain and other tissue samples were harvested to study transduction efficiency. Transduction by the AAV801-PGRNΔ3 vector was confirmed in all brain regions tested by quantifying the presence of vectors and was much higher by a factor of at least 100 compared to transduction by AAV9-PGRNΔ3 vector at the same dose (2e13 vg/kg) (FIG. 20), again demonstrating the superiority of AAV801 for crossing the BBB to transduce brain, including deep brain structures, compared to AAV9. Spinal cord was also more highly transduced by AAV801-PGRNΔ3 than AAV9-PGRNΔ3 by a factor of about 10. Transduction by the AAV801-PGRNΔ3 vector was dose responsive, but both doses resulted in widespread distribution of vector throughout the brain. In other, especially non-neural tissues, the VGC for AAV801-PGRNΔ3 was lower or equal to that for AAV9-PGRNΔ3. For example, in liver, which tends to be highly transduced by many capsids administered intravenously, VGC for AAV801-PGRNΔ3 about half the VGC for AAV9-PGRNΔ3. Transduction of dorsal root ganglion (DRG) and trigeminal ganglion were relatively low, even at the highest dose tested (2e13 vg/kg).

Consistent with the VGC data, PGRNΔ3 RNA levels produced in test animals treated with AAV801-PGRNΔ3 were 100 to 1000-fold higher compared RNA levels in brain tissue samples taken from test animals that received AAV9-PGRNΔ3 vectors (FIG. 21). PGRNΔ3 RNA levels were particularly high in frontal and temporal cortex (at least 5-fold higher than that from the MfHPRT house keeping gene) and thalamus in comparison to other brain areas such as amygdala or hippocampus and were also high in spinal cord. By contrast, in liver, PGRNΔ3 RNA levels were low, despite relatively high transduction determined by quantifying vectors (about 0.1 copy vector per copy of cellular MfHPRT gene), again suggesting the effect of restricting transgene expression to neuronal cells by using the synapsin promoter. In DRG and trigeminal ganglion, PGRNΔ3 RNA levels resulting from AAV801-PGRNΔ3 transduction were equivalent to that from AAV9-PGRNΔ3 and lower than most brain areas. PGRNΔ3 RNA expression was dose responsive, but both doses resulted in similarly widespread transgene expression throughout the brain.

RNA expression from the PGRNΔ3 transgene was also analyzed by in situ hybridization (ISH). As shown in FIGS. 22A-22G, broad and robust RNA expression was detected in multiple brain regions affected by FTD from test animals administered AAV801-PGRNΔ3 at the 2e13 vg/kg dose, including motor cortex, entorhinal cortex, hippocampal pyramidal cells, thalamus, and dentate nucleus. A similar pattern was observed in test animals receiving the lower 5e12 vg/kg dose, although with diminished frequency and intensity of neuronal staining. Substantial RNA expression from the AAV801-PGRNΔ3 vector was also observed in spinal cord. In test animals that received AAV9-PGRNΔ3 vector at a dose of 2e13 vg/kg, RNA expression was observed by ISH in DRG, spinal cord, and trigeminal ganglia, but no positively staining neurons were detected in brain. Additionally, no RNA expression was detected by ISH in peripheral sympathetic ganglia, e.g., paravertebral ganglia or GI tract neurons.

PGRNΔ3 protein was highly expressed in multiple brain regions from test animals that received 2e13 vg/kg of the AAV801-PGRNΔ3 vector, exceeding the levels of progranulin naturally occurring the brains of the test animals (FIG. 23). For example, in frontal cortex the PGRNΔ3 protein level was about 500 ng per gram of brain tissue, which was about 27-fold higher than the concentration of endogenous macaque progranulin. In temporal cortex, PGRNΔ3 protein level was about 30 ng/g brain tissue, or about 2-fold higher than endogenous progranulin, and was about 40-fold higher than the level of macaque progranulin in spinal cord. Peripherally, PGRNΔ3 levels were about 10 ng/g of DRG tissue, which is lower than endogenous macaque progranulin (about 50 ng/g tissue). PGRNΔ3 protein was also detectable from the AAV801-PGRNΔ3 vector administered at the lower dose (5e12 vg/kg) in some brain regions, including frontal cortex, thalamus, and spinal cord, whereas no protein was detected from the AAV9-PGRNΔ3 vector even at the higher dose (2e13 vg/kg). Finally, no PGRNΔ3 protein from AAV801-PGRNΔ3 was detected in liver, despite the presence of 100 ng/g tissue of endogenous progranulin, again confirming restricted tissue expression by the synapsin promoter. PGRNΔ3 protein expression was also analyzed by immunohistochemistry (ICH) in a number of brain sections. The staining pattern matched that for transgene mRNA determined using ISH, suggesting lack of significant uptake of PGRNΔ3 protein into cells that did not express the vector mRNA.

### Example 7

### Quantitation of Vector-Derived PGRN in NHP Samples

An immunoaffinity liquid chromatography mass spectrometry (LCMS) assay was developed to quantitate the amount of human PGRN protein expressed in tissue or biofluid samples from cynomolgus monkeys treated with AAV vectors for expressing PGRNΔ3. In the method, tissue or biofluid samples from NHP test animals treated with PGRNΔ3 vector are mixed with lysis buffer to release proteins from cells. Polyclonal antibody specific for PGRN is then used to immunoprecipitate endogenous full-length monkey PGRN and truncated human PGRN including, for example, 43 and post-translationally modified 44) produced as a result of vector treatment. After removing non-immunoprecipitated proteins, the immunoprecipitated PGRN proteins are digested with S. aureus V8 GluC protease, which cleaves proteins to the C-terminus of aspartic or glutamic acid residues. In both full-length human and cynomolgus PGRN, GluC cleaves between amino acid residue numbers 576 (E) and 577 (A) to release a 17 amino acid long C-terminal peptide, whereas GluC cleavage of A3 truncated PGRN releases a 14 amino acid long. Mass spectrometry is then be used to distinguish the masses of the longer endogenous full-length monkey PGRN peptide from the shorter human PGRNΔ3 peptide.

The immunoaffinity LCMS assay described above was used to quantify the amount of truncated PGRN protein in pooled CSF samples from NHP test animals treated with AAV801-PGRNΔ3 vector as described in Example 6. Unexpectedly, the predominant truncated C-terminal peptide in the samples corresponded not to the 14 amino acid long peptide (577-590) that would be produced by digesting human PGRNΔ3 with GluC, but instead to the 13 amino acid long peptide (577-589) that would be released by digesting human PGRN with a 4 amino acid C-terminal truncation, i.e., PGRNΔ4. This observation suggests that after administering AAV801-PGRNΔ3 vector to the NHP test animals, the transduced brain cells expressed human PGRNΔ3 protein, which was then modified, possibly intracellularly before secretion, or extracellularly after secretion, or both, to remove the C-terminal arginine (corresponding to position 590 in full-length human PGRN), to produce PGRNΔ4 protein as the predominant human truncated PGRN species in the samples.

TABLE OF SEQUENCES

| SEQ | | |
|---|---|---|
| ID | | |
| NO. | Description | Sequence |
| 1 | AAV801 VP1 | MAADGYLPDWLEDNLSEGIREWWALKPGAPQPKANQQHQDNARGLVLPGYKYLGPGNGLD |
| | capsid protein | KGEPVNAADAAALEHDKAYDQQLKAGDNPYLKYNHADAEFQERLKEDTSFGGNLGRAVFQ |
| | amino acid | AKKRLLEPLGLVEEAAKTAPGKKRPVEQSPQEPQSSAGIGKSGAQPAKKRLNFGQTGDTE |
| | sequence | SVPDPQPIGEPPAAPSGVGSLTMASGGGAPVADNNEGADGVGSSSGNWHCDSQWLGDRVI |
| | | TTSTRTWALPTYNNHLYKQISNSTSGGSSNDNAYFGYSTPWGYFDFNRFHCHESPRDWQR |
| | | LINQNWGFRPKRLNFKLFNIQVKEVIDNNGVKTIANNLTSTVQVFTQADYQLPYVLGSAH |
| | | EGCLPPFPADVFMIPQYGYLTLNDGSQAVGRSSFYCLEYFPSQMLRTGNNFQFSYEFENV |
| | | PFHSSYAHSQSLDRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASEKEGEDRFFPLSGS |
| | | LIFGKQGTGRDNVDADKVMITNEEEIKTTNPVATESYGQVATNHQSPINGAVHLYAQAQT |
| | | GWVQNQGILPGMVWQDRDVYLQGPIWAKIPHTDQNFHPSPLMGGFGMKHPPPQILIKNTP |
| | | VPADPPTAFNKDKLNSFITQYSTGQVSVEIEWELQKEQSKRWNPEIQYTSNYYKSANVEF |
| | | AVNTEGVYSEPRPIGTRYLTRNL |
| 2 | AAV801 VP2 | TAPGKKRPVEQSPQEPQSSAGIGKSGAQPAKKRLNFGQTGDTESVPDPQPIGEPPAAPSG |
| | capsid protein | VGSLTMASGGGAPVADNNEGADGVGSSSGNWHCDSQWLGDRVITTSTRTWALPTYNNHLY |
| | amino acid | KQISNSTSGGSSNDNAYEGYSTPWGYFDENREHCHFSPRDWQRLINNNWGFRPKRLNFKL |
| | sequence | FNIQVKEVTDNNGVKTIANNLTSTVQVFTGSDYQLPYVLGSAHEGCLPPFPADVFMIPQY |
| | | GYLTLNGGSQAVGRSSFYCLEYFPSQMLRTGNNFQFSYEFENVPFHSSYAHSQSLDRLMN |
| | | PLIDQYLYYLSKTINGSGQNQQTLKFSVAGPSNMAVQGRNYIPGPSYRQQRVSTTVTQNN |
| | | NSEFAWPGASSWALNGRNSLMNPGPAMASEKEGEDRFFPLSGSLIFGKQGTGRDNVDADK |
| | | VMICNEEEIKTTNPVATESYGQVATNHQSPLNGAVHLYAQAQTGWVQNQGILPGMVWQDR |
| | | DVYLQGPIWAKIPHTGGNFHPSPLMGGFGMKHPPPQILIKNTPVPADDPTAFNKDKLNSF |
| | | ITQYSTGQVSVEIEWELQKEWSKRWNPEIQYTSNYYKSNNVEFAVNTEGVYSEPRPIGTR |
| | | YLTRNL |
| 3 | AAV801 VP3 | MASGGGAPVADNNEGADGVGSSSGNWHCDSQWLGDRVITTSTRTWALPTYNNHLYKQISN |
| | capsid protein | STSGGSSNDNAYFGYSTPWGYFDFNRFHCEFSPRDWQRLINNNWGFRPKRLNFKLFNIQV |
| | amino acid | KEVTDNNGVKTIANNLTSTVQVFTDGDYQLPYVLGSAHEGCLPPFPADVFMIPQYGYLTL |
| | sequence | NDGSQAVGRSSFYCLEYFPSQMLRTGNNFQFSYEFENVPFHSSYAHSQSLDRLMNPLIDQ |
| | | YLYYLSKTINGSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDNVDADKVMITN |
| | | EEEIKTTNPVATESYGQVATNHQSPLNGAVHLYAQAQTGWVQNQGILPGMVWQDRDVYLQ |
| | | GPIWAKIPHIDGNFHPSPLMGGFGMKHPPPQILIKNTPVPADDPTAFNKDKLNSFITQYS |
| | | TGQVSVEIEWELQKENSKRWNPEIQYTSNYYKSNNVEFAVNTEGVYSEPRPIGTRYLTRN |
| | | L |
| | | Key for SEQ ID NOs: 1-3 |
| | | Bold Italic Underline: post translationally hydrolyzed N terminal |
| | | residue |
| | | Bold Italic: acetylated alanine |
| | | Light blue: potential cleavage site |
| | | Red: potential deamidation site |
| | | Light green: potential isomerization site |
| | | Dark blue: potential methionine oxidation site |
| 4 | AAV-PHP.B VP1 | MAADGYLPDWLEDNLSEGIREWWALKPGAPQPKANQQHQDNARGLVLPGYKYLGPGNGLD |
| | capsid protein | KGEPVNAADAAALEHDKAYDQQLKAGDNPYLKYNHADAEFQERLKEDTSFGGNLGRAVFQ |
| | amino acid | AKKRLLEPLGLVEEAAKTAPGKKRPVEQSPQEPDSSAGIGKSGAQPAKKRLNFGQTGDTE |
| | sequence | SVPDPQPIGEPPAAPSGVGSLTMASGGGAPVADNNEGADGVGSSSGNWHCDSQWLGDRVI |
| | | TTSTRTWALPTYNNHLYKQISNSTSGGSSNDNAYFGYSTPWGYFDENRFHCHFSPRDWQR |
| | | LINNNWGFRPKRLNFKLFNIQVKEVTDNNGVKTIANNLTSTVQVFTDSDYQLPYVLGSAH |
| | | EGCLPPFPADVFMIPQYGYLTLNDGSQAVGRSSFYCLEYFPSQMLRTGNNFQFSYEFENV |
| | | PFHSSYAHSQSLDRLMNPLIDQYLYYLSRTINGSGQNQQTLKFSVAGPSNMAVQGRNYIP |
| | | GPSYRQQRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQTLAVPFKAQAQT |
| | | GWVQNQGILPGMVWQDRDVYLQGPIWAKIPHTDGNFHPSPLMGGFGMKHPPPQILIKNTP |
| | | VPADPPTAFNKDKINSFITQYSTGQVSVEIEWELQKENSKRWNPEIQYTSNYYKSNNVEF |
| | | AVNTEGVYSEPRPIGTRYLTRNL |
| 5 | 5' Inverted | TTGGCCACTCCCTCTCTGCGCGCTCGCTCGCTCACTGAGGCCGCCCGGGCAAAGCCCGGGCGTCGGG |
| | Terminal Repeat | CGACCTTTGGTCGCCCGGCCTCAGTGAGCGAGCGAGCGCGCAGAGAGGGAGTGGCCAACTCCATCAC |
| | (5 '-ITR) | TAGGGGTTCCT |
| | nucleotide | |
| | sequence (flop | |
| | configuration) | |
| 6 | Human synapsin | ctgagttgaatatcaacactacaaaccgagtatctgcagagggccctgcgtatgagtgcaagtgggt |
| | gene promoter | tttaggaccaggatgaggcggggtgggggtgcctacctgacgaccgaccccgacccactggacaagc |
| | (hSyn pro) | acccaacccccattccccaaattgcgcatcccctatcagagagggggaggggaaacaggatgcggcg |
| | | aggcgcgtgcgcactgccagcttcagcaccgcggacagtgccttcgcccccgcctggcggcgcgcgc |
| | | caccgccgcctcagcactgaaggcgcgctgacgtcactcgccggtcccccgcaaactccccttcccg |
| | | gccaccttggtcgcgtccgcgccgccgccggcccagccggaccgcaccacgcgaggcgcgagatagg |
| | | ggggcacgggcgcgaccatctgcgctgcggcgccggcgactcagcgctgcctc |
| 7 | 5' untranslated | agtctgcggtgggcagcggaggagtcgtgtcgtgcctgagagcgcagctgtgctcctgggcaccgcg |
| | region from | cagtccgcccccgcggctcctggccagaccacccctaggaccccctgccccaagtcgcagcc |
| | human synapsin | |
| | 1 gene (5' UTR | |
| | SYN1) | |
| 8 | Human | atgtggaccctgctgagctgggtggccttaacagcagggctggtggctggaacgcgctgcccagatg |
| | progranulin | gtcagttctgccctgtggcctgctgcctggaccccggaggagccagctacagctgctgccgtcccct |
| | De13 | tctggacaaatggcccacaacactgagcacgcatctgggtggcccctgccaggttgatgcccactgc |
| | polypeptide | tctgccggccactcctgcatctttaccgtctcagggacttccagttgctgccccttcccagaggccg |
| | (PGRNA3) | tggcatgcggggatggccatcactgctgcccacggggcttccactgcagtgcagaccggcgatcctg |
| | encoding | cttccaaagatcaggtaacaactccgtgggtgccatccagtgccctgatagtcagttcgaatgcccg |
| | nucleotide | gacttctccacgtgctgtgttatggtcgatggctcctgggggtgctgccccatgccccaggcttcct |
| | sequence | gctgtgaagacagggtgcactgctgtccgcacggtgccttctgcgacctggttcacacccgctgcat |
| | (hGRNA3) | cacacccacgggcacccaccccctggcaaagaagctccctgcccagaggactaacagggcagtggcc |
| | | ttgtccagctcggtcatgtgtccggacgcacggtcccggtgccctgatggttctacctgctgtgagc |
| | | tgcccagtgggaagtatggctgctgcccaatgcccaacgccacctgctgctccgatcacctgcactg |
| | | ctgcccccaagacactgtgtgtgacctgatccagagtaactgcctctccaaggagaacgctaccacg |
| | | gacctcctcactaagctgcctgcgcacacagtgggggatgtgaaatgtgacatggacgtgagctgcc |
| | | cagatggctatacctgctgccgtctacagtcgggggcctggggctgctgcccttttacccaggctgt |
| | | gtgctgtgaggaccacatacactgctgtcccgcggggtttacgtgtgacacgcagaagggtacctgt |
| | | gaacaggggccccaccaggtgccctggatcgagaaggccccagctcacctcagcctgccagacccac |
| | | aagccttgaagagagatgtcccctgtgataatgtcagcagctgtccctcctccgatacctgctgcca |
| | | actcacgtctggggagtggggctgctgtccaatcccagaggctgtctgctgctcggaccaccagcac |
| | | tgctgcccccagggctacacgtgtgtagctgaggggcagtgtcagcgaggaagcgagatcgtggctg |
| | | gactggagaagatgcctgcccgccgggcttccttatcccaccccagagacatcggctgtgaccagca |
| | | caccagctgccccggtggggcagacctgctgcccgagcctggggggagctgggcctgctgccagttg |
| | | ccccatgctgtgtgctgcgaggatcgccaccactgctgcccggctggctacacctgcaacgtgaagg |
| | | ctcgatcctgcgagaaggaagtcgtctctgcccagcctgccaccttcctggcccgtagccctcacgt |
| | | gggtgtgaaggacgtggagtgtggggaaggacacttctgccatgataaccagacctgctgccgagac |
| | | aaccgacagggctgggcctgctgtccctaccgccagggcgtctgttgtgctgatcggcgccactgct |
| | | gtcctgctggcttccgctgcgcagccaggggtaccaagtgtttgcgcagggaggccccgcgctggga |
| | | cgcccctttgagggacccagccttgagatga |
| 9 | bGH polyA | ctgtgccttctagttgccagccatctgttgtttgcccctcccccgtgccttccttgaccctggaagg |
| | nucleotide | tgccactcccactgtcctttcctaataaaatgaggaaattgcatcgcattgtctgagtaggtgtcat |
| | sequence | tctattctggggggtggggtggggcaggacagcaagggggaggattcggaagacaatagcaggcatg |
| | | c |
| 10 | bGH polyA | CGACTGTGCCTTCTAGTTGCCAGCCATCTGTTGTTTGCCCCTCCCCCGTGCCTTCCTTGACCCTGGA |
| | nucleotide | AGGTGCCACTCCCACTGTCCETTCCTAATAAAATGAGGAAATTGCATCGCATTGECTGAGTAGGTGT |
| | sequence | CATTCTATTCTGGGGGGTGGGGTGGGGCAGGACAGCAAGGGGGAGGATTGGGAAGACAATAGCAGGC |
| | | ATGCTGGGGATGCGGTGGGCTCTATGG |
| 11 | TBP intron | gtaatctcagtaccttgggaggccaaggtcgaaggatcactcaaagctaggagctcaagaccaacct |
| | stuffer | gggtaacaaaacaagtccctgttgctacaattagctggtcaaggtggcaagtgcctgtagtcccagc |
| | | tacttgggaggctgaggcgggagggtcacttgagtccagaagttaaaggctacattcgagaccctgt |
| | | ctcaaagaaaggaagcagggagggagaattgctgtagctgccaaagatagtgattcctctgttgaat |
| | | ctgggcaaagtgaattgaaaactagacttcagtgctggaagttactgcaggtgtagtggaaatagca |
| | | agtgaactagaattagaagtgaacctgaagttgtgactgaattgctgcaattttttgataaaacctg |
| | | aacagttgaggagttgcttcttgtgagtaagcaaagaaagtggtttcttgagatagaattgactcct |
| | | gctgaagaactgttgactttagaatattacaaaaacttagttgataaagcagcagcagggtttgaga |
| | | ggattgactccaattttgaaataagttctagtgtgggtaaattgctgtcaaatagtattaattgcta |
| | | cagagacaacttcagtgaaaggaagagtcagtcagcgcagcgcttgtggcaaacttcgtcagtctta |
| | | ttttaagaaattgccacagctaccaccctgatcagtcagcagccaacaacaacgagccaagatcctc |
| | | tgtcagcaaaaagattttgatttgctgcaggctcacaagattgttagcaattttagcaataaagcaa |
| | | ttttaaattaagttatatacaaattattagacaaattgctattgcacacttaatagactttagtgct |
| | | aacaaaacttttgtaggcactgggaaaccaaaaaattgttgccgcttgctttattgagttggtctgg |
| | | aacctaacctgtagtatctccgaggtttgcctgtatctttaattgtaatttgtccttcacagctagc |
| | | a |
| 12 | 3' Inverted | aggaacccctagtgatggagttggccactccctctctgcgcgctcgctcgctcactgaggccgggcg |
| | Terminal Repeat | accaaaggtcgcccgacgcccgggctttgcccgggcggcctcagtgagcgagcgagcgcgcagagag |
| | (3'-ITR) | ggagtggccaa |
| | nucleotide | |
| | sequence (flip | |
| | configuration) | |
| 13 | 3' Inverted | AGGAACCCCTAGTGATGGAGTTGGCCACTCCCTCTCTGCGCGCTCGCTCGCTCACTGAGGCCGGGCG |
| | Terminal Repeat | ACCAAAGGTCGCCCGACGCCCGGGCTTTGCCCGGGGGGCCTCAGTGAGCGAGCGAGCGCGCAGAGAG |
| | (3' -ITR) | GGAGTGGCCAA |
| | nucleotide | |
| | sequence (flop | |
| | configuration) | |
| 14 | Human | MWTLVSWVALTAGLVAGTRCPDGQFCPVACCLDPGGASYSCCRPLLDKWPTTLSRHLGGP |
| | progranu_in | CQVDAHCSAGHSCIFTVSGTSSCCPFPEAVACGDGHHCCPRGFHCSADGRSCFQRSGNNS |
| | De13 | VGAIQCPCSQFECPDFSTCCVMVDGSWGCCPMPQASCCEDRVHCCPEGAFCDLVHIRCIT |
| | polypeptide | PTGTHPLAKKLPAQRTNRAVALSSSVMCPDARSRCPDGSTCCELPSGKYGCCPMPNATCC |
| | (PGRNA3) | SDHLHCCPQDTVCDLIQSKCLSKENATTDLLTKLPAHTVGDVKCDMEVSCPDGYTCCRLQ |
| | | SGAWGCCPFTQAVCCEDHIHCCPAGFTCDTQKGTCEQGPEQVPWMEKAPAHLSLPDRQAL |
| | | KRDVPCDNVSSCPSSDTCCQLTSGEWGCCPIPEAVCCSDEQHCCPQGYTCVAEGQCQRGS |
| | | EIVAGLEKMPARRASLSHPRDIGCDQHTSCPVGQTCCPSLGGSWACCQLPHAVCCEDRQH |
| | | CCPAGYTCNVKARSCEKEVVSAQPATFLARSPHVGVKDVECGEGHFCHDNQTCCRDKRQG |
| | | WACCPYRQGVCCADRRHCCPAGERCAARGTKCLRREAPRWDAPLRDPALR |
| | | Key |
| | | Purple: potential N-Linked glycosylation consensus sequence site |
| | | Light blue: potential cleavage site |
| | | Red: potential deamidation site |
| | | Light green: potential isomerization site |
| | | Dark blue: potential methionine oxidation site |
| | | Bold: expected N-terminal pyroglutamic acid |
| 15 | Human | ATGTGGACCCTGGTGAGCTGGGTGGCCTTAACAGCAGGGCTGGTGGCTGGAACGCGGTGC |
| | progranulin | CCAGATGGTCAGTTCTGCCCTGTGGCCTGCTGCCTGGACCCCGGAGGAGCCAGCTACAGC |
| | precursor | TGCTGCCGTCCCCTTCTGGACAAATGGCCCACAACACTGAGCAGGCATCTGGGTGGCCCC |
| | polypeptide | TGCCAGGTTGATGCCCACTGCTCTGCCGGCCACTCCTGCATCTTTACCGTCTCAGGGACT |
| | (full-length) | TCCAGTTGCTGCCCCTTCCCAGAGGCCGIGGCATGGGGGGATGGCCATCACTGCTGCCCA |
| | mRNA sequence; | CGGGGCTTCCACTGCAGTGCAGACGGGCGATCCTGCTTCCAAAGATCAGGTAACAACTCC |
| | NCBI Reference | GTGGGTGCCATCCAGTGCCCTGATAGTCAGTTCGAATGCCCGGACTTCTCCACGTGCTGT |
| | Sequence: | GTTATGGTCGATGGCTCCTGGGGGTGCTGCCCCATGCCCCAGGCTTCCTGCTGTGAAGAC |
| | NM_002087.4 | AGGGTGCACTGCTGTCCGCACGGTGCCTTCTGCGACCTGGTTCACACCCGCTGCATCACA |
| | | CCCACGGGCACCCACCCCCTGGCAAAGAAGCTCCCTGCCCAGAGGACTAACAGGGCAGTG |
| | | GCCTTGTCCAGCTCGGTCATGTGTCCGGACGCACGGTCCCGGTGCCCTGATGGTTCTACC |
| | | TGCTGTGAGCTGCCCAGTGGGAAGTATGGCTGCTGCCCAATGCCCAACGCCACCTGCTGC |
| | | TCCGATCACCTGCACTGCTGCCCCCAAGACACTGTGTGTGACCTGATCCAGAGTAAGTGC |
| | | CTCTCCAAGGAGAACGCTACCACGGACCTCCTCACTAAGCTGCCTGCGCACACAGTGGGG |
| | | GATGTGAAATGTGACATGGAGGTGAGCTGCCCAGATGGCTATACCTGCTGCCGTCTACAG |
| | | TCGGGGGCCTGGGGCTGCTGCCCTTTTACCCAGGCTGTGTGCTGTGAGGACCACATACAC |
| | | TGCTGTCCCGCGGGGTTTACGTGTGACACGCAGAAGGGTACCTGTGAACAGGGGCCCCAC |
| | | CAGGTGCCCTGGATGGAGAAGGCCCCAGCTCACCTCAGCCTGCCAGACCCACAAGCCTTG |
| | | AAGAGAGATGTCCCCTGTGATAATGTCAGCAGCTGTCCCTCCTCCGATACCTGCTGCCAA |
| | | CTCACGTCTGGGGAGTGGGGCTGCTGTCCAATCCCAGAGGCTGTCTGCTGCTCGGACCAC |
| | | CAGCACTGCTGCCCCCAGGGCTACACGTGTGTAGCTGAGGGGCAGTGTCAGCGAGGAAGC |
| | | GAGATCGTGGCTGGACTGGAGAAGATGCCTGCCCGCCGGGCTICCTTATCCCACCCCAGA |
| | | GACATCGGCTGTGACCAGCACACCAGCTGCCCGGTGGGGCAGACCTGCTGCCCGAGCCTG |
| | | GGTGGGAGCTGGGCCTGCTGCCAGTTGCCCCATGCTGTGTGCTGCGAGGATCGCCAGCAC |
| | | TGCTGCCCGGCTGGCTACACCTGCAACGTGAAGGCTCGATCCTGCGAGAAGGAAGTGGTC |
| | | TCTGCCCAGCCTGCCACCTTCCTGGCCCGTAGCCCTCACGTGGGTGTGAAGGACGTGGAG |
| | | TGTGGGGAAGGACACTTCTGCCATGATAACCAGACCTGCTGCCGAGACAACCGACAGGGC |
| | | TGGGCCTGCTGTCCCTACCGCCAGGGCGTCTGTTGTGCTGATCGGCGCCACTGCTGTCCT |
| | | GCTGGCTTCCGCTGCGCAGCCAGGGGTACCAAGTGTTTGCGCAGGGAGGCCCCGCGCTGG |
| | | GACGCCCCTTTGAGGGACCCAGCCTTGAGACAGCTGCTGTGA |
| 16 | Human | MWTLVSWVALTAGLVAGTRCPDGQFCPVACCLDPGGASYSCCRPLLDKWPTTLSRHEGGP |
| | progranu_in | CQVDAHCSAGHSCIFTVSGTSSCCPFPEAVACGDGHHCCPRGFHCSADGRSCFQRSGNNS |
| | precursor | VGAIQCPDSQFECPDFSTCCVMVDGSWGCCPMPQASCCEDRVHCCPHGAFCDLVHTRCIT |
| | polypeptide | PTGTHPLAKKLPAQRTNRAVALSSSVMCPDARSRCPDGSTCCELPSGKYGCCPMPNATCC |
| | (full-length); | SDHLHCCPQDTVCDLIQSKCLSKENATTDLLTKLPAHTVGDVKCDMEVSCPDGYTCCRLQ |
| | NCBI Reference | SGAWGCCPFTQAVCCEDHIHCCPAGFTCDTQKGTCEQGPHQVPWMEKAPAHLSLPDPQAL |
| | Sequence: | KRDVPCDNVSSCPSSDTCCQLTSGEWGCCPIPEAVCCSDHQHCCPQGYTCVAEGQCQRGS |
| | NP_002078.1 | EIVAGLEKMPARRASLSHPRDIGCDQHTSCPVGQTCCPSLGGSWACCQLPHAVCCEDRQH |
| | | CCPAGYTCNVKARSCEKEVVSAQPATFLARSPHVGVKDVECGEGHFCHDNQTCCRDNRQG |
| | | WACCPYRQGVCCADRRHCCPAGFRCAARGTKCLRREAPRWDAPLRDPALRQLL |
| 17 | Human | MWTLVSWVALTAGLVAG |
| | progranulin | |
| | precursor | |
| | polypeptide | |
| | (signal | |
| | peptide); NCBI | |
| | Reference | |
| | Sequence: | |
| | NP_002078.1 | |
| 18 | Human | TRCPDGQFCPVACCLDPGGASYSCCRPLLDKWPTTLSRHLGGPCQVDAHCSAGHSCIFTV |
| | progranulin | SGTSSCCPFPEAVACGDGHHCCPRGFHCSADGRSCFQRSGNNSVGAIQCPDSQFECPDFS |
| | precursor | TCCVMVDGSWGCCPMPQASCCEDRVHCCPHGAFCDLVHTRCITPTGTHPLAKKLPAQRTN |
| | polypeptide | RAVALSSSVMCPDARSRCPDGSTCCELPSGKYGCCPMPNATCCSDHLHCCPQDTVCDLIQ |
| | (mature | SKCLSKENATTDLLTKLPARTVGDVKCDMEVSCPDGYTCCRLQSGAWGCCPFTQAVCCED |
| | polypeptide); | HIHCCPAGFTCDTQKGTCEQGPHQVPWMEKAPAHLSLPDPQALKRDVPCDNVSSCPSSDT |
| | NCBI Reference | CCQLTSGEWGCCPIPEAVCCSDHQHCCPQGYTCVAEGQCQRGSEIVAGLEKMPARRASLS |
| | Sequence: | HPRDIGCDQHTSCPVGQTCCPSLGGSWACCQLPHAVCCEDRQHCCPAGYTCNVKARSCEK |
| | NP_002078.1 | EVVSAQPATFLARSPHVGVKDVECGEGHFCHDNQTCCRDNRQGWACCPYRQGVCCADRRH |
| | | CCPAGFRCAARGTKCLRREAPRWDAPLRDPALRQLL |
| 19 | #249-Syn-PGRNA3 | ttggccactccctctctgcgcgctcgctccctcactgaggccgcccgggcaaagcccgggcgtcggg |
| | vector | cgacctttggtcgccccggcctcagtgagcgagcgagcgccagagagggagtggccaactccatcac |
| | nucleotide | taggggttccttaccgcgtcaattcgcgcctgagttgaatatcaacactacaaaccgagtatctgca |
| | sequence (5'- | gagggccctgcgtatgagtgcaagtgggttttaggaccaggatgaggcggggtgggggtgcctacct |
| | ITR to 3'-ITR) | gacgaccgaccccgacccactggacaagcacccaacccccattccccaaattgcgcatcccctatca |
| | | gagagggggaggggaaacaggatgcggcgaggcgcgtgcgcactgccagcttcagcaccgcggacag |
| | | tgccttcgcccccgcctggcggcgcgcgccaccgccgcctcagcactgaaggcqcgctgacgtcact |
| | | cgccggtcccccgcaaactccccttcccggccaccttggtcgcgtccgcgccgccgccggcccagcc |
| | | ggaccgcaccacccgaggcgcgagataggggggcacgggcgcgaccatctgcgctgcggcgccggcg |
| | | actcagcgctgcctcagtctgccgtgggcagcggaggagtcgtgtcgtgcctgagagcgcagctgtg |
| | | ctcctgggcaccgcgcagtccgcccccgcggctcctggccagaccacccctaggaccccctgcccca |
| | | agtcgcagccttcgaattctgcagtcgacctttaaacgctagcaagcttccaccatctggaccctgg |
| | | tgagctgggtggccttaacagcagggctggtggctggaacgcggtgcccagatggtcagttctgccc |
| | | tgtggcctgctgcctggaccccggaggagccagctacagctgctgccgtccccttctggacaaatgg |
| | | cccacaacactgagcaggcatctgggtggcccctgccaggttgatgcccactgctctgccggccact |
| | | cctgcatctttaccgtctcagggacttccagttgctgccccttcccagaggccgtggcatgcgggga |
| | | tggccatcactgctgcccacggggcttccactgcagtgcagacgggcgatcctgcttccaaagatca |
| | | ggtaacaactccgtgggtgccatccagtgccctgatagtcagttcgaatgcccggacttctccacgt |
| | | gctgtgttatggtcgatggctcctgggggtgctgccccatgccccaggcttcctgctgtgaagacag |
| | | ggtgcactgctgtccgcacggtgccttctgcgacctggttcacacccgctgcatcacacccacgggc |
| | | acccaccccctggcaaagaagctccctgcccagaggactaacagggcagtggccttgtccagctcgg |
| | | tcatgtgtccggacgcacggtcccggtgccctgatggttctacctgctgtgagctgcccagtgggaa |
| | | gtatggctgctgcccaatgcccaacgccacctgctgctccgatcacctgcactgctgcccccaagac |
| | | actgtgtgtgacctgatccagagtaagtgcctctccaaggagaacgctaccacggacctcctcacta |
| | | agctgcctgcgcacacagtgggggatgtgaaatgtgacatggaggtgagctgcccagatggctatac |
| | | ctgctgccgtctacagtcgggggcctggggctgctgcccttttacccaggctgtgtcctgtgaggac |
| | | cacatacactgctgtcccgcggggtttacgtgtgacacgcagaagggtacctgtgaacaggggcccc |
| | | accaggtgccctggatggagaaggccccagctcacctcagcctgccagacccacaagccttgaagag |
| | | agatgtcccctgtgataatgtcagcagctgtccctcctccgatacctgctgccaactcacgtctggg |
| | | gagtggggctgctgtccaatcccagaggctgtctgctgctcggaccaccagcactgctgcccccagg |
| | | gctacacgtgtgtagctgaggggcagtgtcagcgaggaagcgagatcgtggctggactggagaagat |
| | | gcctgcccgccgcgcttccttatcccaccccagagacatcggctgtgaccagcacaccagctgcccg |
| | | gtggggcagacctgctgcccgaccctgggtgggagctgggcctgctgccagttgccccatgctgtgt |
| | | gctgcgaggatcgccagcactgctgcccggctggctacacctgcaacgtgaaggctcgatcctgcga |
| | | gaaggaagtggtctctgcccagcctgccaccttcctggcccgtagccctcacgtgggtgtgaaggac |
| | | gtggagtgtggggaaggacacttctgccatgataaccagacctgctgccgagacaaccgacagggct |
| | | gggcctgctgtccctaccgccagggcgtctgttgtgctgatcggcgccactgctgtcctgctggctt |
| | | ccgctgcgcagccaggggtaccaagtgtttgcgcagggaggccccgcgctgggacgcccctttgagg |
| | | gacccagccttgagatgagcggccgcgttaacctgtgccttctagttgccagccatctgttgtttgc |
| | | ccctcccccgtgccttccttgaccctggaaggtgccactcccactgtcctttcctaataaaatgagg |
| | | aaattgcatcgcattgtctgagtaggtgtcattctattctggggggtggggtggggcaggacagcaa |
| | | gggggaggattgggaagacaatagcaggcatgcaagcttctaatctcagtaccttgggaggccaagg |
| | | tggaaggatcactcaaagctaggagctcaagaccaacctcggtaacaaaacaagtccctgttgctac |
| | | aattagctggtcaaggtggcaactgcctgtagtcccagctacttgggaggctgaggcgggagggtca |
| | | cttgagtccagaagttaaaggctacattggagaccctgtctcaaagaaaggaagcagggagggagaa |
| | | ttgctgtagctgccaaagatagtgattcctctgttgaatctgggcaaagtgaattgaaaactagact |
| | | tcagtgctggaagttactgcagctgtagtcgaaatagcaagtgaactagaattagaagtgaacctga |
| | | agttgtgactgaattgctgcaattttttgataaaacctgaacagttgaggagttgcttcttgtgagt |
| | | aagcaaagaaagtggtttcttgagatagaattgactcctggtgaagaactgttgactttagaatatt |
| | | acaaaaacttagttgataaagcagcagcagggtttgagaggattgactccaattttgaaataagttc |
| | | tagtgtgggtaaattgctgtcaaatagtattaattgctacagagacaacttcagtgaaaggaagagt |
| | | cagtcagcgcagcgcttgtggcaaacttcgtcagtcttattttaagaaattgccacagctaccaccc |
| | | tgatcagtcagcagccaacaacaacgaggcaagatcctctgtcagcaaaaagattttgatttgctgc |
| | | aggctcacaagattgttagcaattttagcaataaagcaattttaaattaagttatatacaaattatt |
| | | agacaaattgctattgcacacttaatagactttagtgctaacaaaacttttgtaggcactgggaaac |
| | | caaaaaattgttgccgcttgctttattgagttggtctggaacctaacctgtagtatctccgaggttt |
| | | gcctgtatctttaattgtaatttgtccttcacagctagcagtactaggaacccctactgatggagtt |
| | | ggccactccctctctgcgcgctcgctcgctcactgaggccgggcgaccaaaggtcgcccgacgcccg |
| | | ggctttgcccgggcggcctcagtgagcgagcgagcgcgcagagagggagtggccaa |
| 20 | AAV2 ITR (130 | aggaacccctagtgatggagttcgccactccctctctgcgcgctcgctcgctcactgaggccgggcg |
| | bp) | accaaaggtcgcccgacgcccgggctttgcccgggcggcctcagtgagcgagcgagcgcgcag |
| 21 | AAV2 ITR (130 | ctgcgcgctcgctcgctcactgaggccgcccgggcaaagcccgggcgtcgggcgacctttggtcgcc |
| | bp) | cggcctcagtgagcgagcgagcgcgcagagagggagtggccaactccatcactaggggttcct |
| 22 | CMV Enhancer | gacattgattattgactagttattaatagtaatcaattacggggtcattagttcatagcccatatat |
| | and Chicken | ggagttccgcgttacataacttacggtaaatggcccgcctggctgaccgcccaacgacccccgccca |
| | Beta Actin | ttgacgtcaataatgacgtatgttcccatagtaacgccaatagggactttccattgacgtcaatggg |
| | Promoter | tggactatttacggtaaactgcccacttggcagtacatcaagtgtatcatatgccaagtacgccccc |
| | | tattgacgtcaatgacggtaaatggcccgcctggcattatgcccagtacatgaccttatgggacttt |
| | | cctacttggcagtacatctacgtattagtcatcgctattaccatgggtcgaggtgagccccacgttc |
| | | tgcttcactctccccatctcccccccctccccacccccaattttgtatttatttattttttaattat |
| | | ggggcggggcgaggcggagaggtgcggcggcagccaatcagagcggcgcgctccgaaagtttccttt |
| | | tatggcgaggcggcggcggcggcggccctataaaaagcgaagcgcgcgggggcg |
| 23 | CMV Enhancer | gacattgattattgactagttattaatagtaatcaattacggggtcattagttcatagcccatatat |
| | | ggagttccgcgttacataacttacggtaastggcccgcctggctgaccgcccaacgacccccgccca |
| | | ttgacgtcaataatgacgtatgttcccatagtaacgccaatagggactttccattgacgtcaatggg |
| | | tggactatttacggtaaactgcccacttggcagtacatcaagtgtatcatatgccaagtacgccccc |
| | | tattgacgtcaatgacggtaaatggcccgcctggcattatgcccagtacatgaccttatgggacttt |
| | | cctacttggcagtacatctacgtattagtcatcgctattaccatg |
| 24 | Chicken Beta | tcgaggtgagccccacgttctgcttcactctccccatctcccccccctccccacccccaattttgta |
| | Actin Promoter | tttatttattttttaattattttgtgcagcgatgggggcggggggggggggggggsgcgcgccgggg |
| | | gcgcgctccgaaagtttccttttatggcgaggcggcggccgcggcggccctataaaaagcgaagcgc |
| | | gcggcgggcg |
| 25 | SV40 Promoter | gcatctcaattagtcagcaaccatagtcccgcccctaactccgcccatcccgcccctaactccgccc |
| | | agttccgcccattctccgccccatggctgactaattttttttatttatgcagaggccgaggccgcct |
| | | cggcctctgagctattccagaagtagtgaggaggcttttttggaggcctaggcttttgcaaa |
| 26 | SV40 poly (A) | taagatacattgatgagtttggacaaaccacaactagaatgcagtgaaaaaaatgctttatttgtga |
| | Signal | aatttytgatgctattgctttatttgtaaccattataagctgcaataaacaagtt |
| 27 | SV40 poly (A) | aacttgtttattgcagcttataatggttacaaataaagcaatagcatcacaaatttcacaaataaag |
| | Signal | catttttttcactgcattctagttqtggtttgtccaaactcatcaatgtatcttatcatgtctggat |
| | | c |
| 28 | Rabbit Beta | aataaaggaaatttattttcattgcaatagtgtgttggaattttttgtgtctctca |
| | Globir poly (A) | |
| | Signal | |
| 29 | WPRE | aatcaacctctggattacaaaatttgtgasagattgactggtattcttaactatgttgctcctttta |
| | | cgctatgtggatacgctgctttaatgcctttgtatcatgctattgcttcccgtatggctttcatttt |
| | | ctcctccttgtataaatcctggttgctgtctctttatgaggagttgtggcccgttgtcaggcaacgt |
| | | ggcgtcgtqtgcactgtgtttgctgacgcaacccccactggttggggcattgccaccacctgtcagc |
| | | tcctttccgggactttcgctttccccctccctattgccacggcggaactcatcgccgcctgccttgc |
| | | ccgctgctggacaggggctcggctgttgggcactgacaattccgtggtgttgtcggggaaatcatcg |
| | | tcctttccttggctgctcgcctctgttgccacctggattctgcgcgggacgtccttctgctacgtcc |
| | | cttcggccctcaatccagcggaccttccttcccgcggcctgctgccggctctgcggcctcttccgcg |
| | | tcttcgccttcgccctcagacgagtcggatctccctttgggccgcctccccgc |
| 30 | GRN probe | CGTGTGACACGCAGAAGGGT |
| | primer | |
| 31 | GRN forward | CATACACTGCTGTCCCGCGG |
| | primer | |
| 32 | GRN reverse | AGGTGAGCTGGGGCCTTCTC |
| | primer | |

1. A recombinant adeno-associated virus (AAV) vector comprising a nucleotide sequence encoding a human progranulin (PGRN) polypeptide variant, wherein the PGRN polypeptide variant is a carboxy-terminal truncation variant that lacks one or more amino acids otherwise present in full-length wild-type human PGRN polypeptide, such that the variant PGRN polypeptide has reduced binding to human sortilin receptor (SORT1) protein compared to full-length wild-type human PGRN polypeptide, wherein the PGRN polypeptide variant lacks the final three or four carboxy-terminal amino acids that are present in full-length wild-type human PGRN polypeptide, or wherein the amino acid sequence of the PGRN polypeptide variant comprises the amino acid sequence of SEQ ID NO:14, SEQ ID NO:16, or either SEQ ID NO:14 or SEQ ID NO:16 with the amino-terminal signal sequence of SEQ ID NO:17 removed.

2. (canceled)

3. (canceled)

4. (canceled)

5. (canceled)

6. The AAV vector of claim 1, wherein the nucleotide sequence encoding said PGRN polypeptide variant, is a codon-optimized nucleotide sequence, wherein the codon-optimized nucleotide sequence has a reduced number of CpG di-nucleotides compared to a wild-type nucleotide sequence encoding the PGRN polypeptide variant, wherein the nucleotide sequence encoding the PGRN polypeptide variant has 1-50, 1-45, 1-40, 1-35, 1-30, 1-25, 1-20, 1-15, 1-10, or 1-5 fewer CpG di-nucleotides compared to a wild-type nucleotide sequence encoding the PGRN polypeptide, variant, or wherein the nucleotide sequence encoding the PGRN polypeptide variant is devoid of any CpG di-nucleotides.

7. (canceled)

8. (canceled)

9. The AAV vector of claim 6, wherein the wild-type nucleotide sequence encoding the PGRN polypeptide is comprised by the nucleotide sequence of SEQ ID NO: 8 or SEQ ID NO:15.

10. (canceled)

11. The AAV vector of claim 1, wherein the nucleotide sequence encoding the PGRN polypeptide is at least 95% identical to the nucleotide sequence of SEQ ID NO:8 or SEQ ID NO: 15 or is identical to the nucleotide sequence of SEQ ID NO:8 or SEQ ID NO:15.

12. (canceled)

13. The AAV vector of claim 1, wherein the vector comprises at least one AAV inverted terminal repeat (ITR) or wherein the nucleotide sequence comprises a wild-type or modified AAV inverted terminal repeat (ITR).

14. (canceled)

15. The AAV vector of claim 13, wherein
(i) the nucleotide sequence of the ITR is modified to reduce or eliminate the ability of the ITR to undergo terminal resolution,
(ii) the nucleotide sequence of the ITR is modified to reduce or eliminate the ability of the ITR to support packaging into a capsid,
(iii) the nucleotide sequence of the ITR is modified to alter the activity of the D region,
(iv) the ITR is an AAV2 ITR,
(v) the ITR comprises the nucleotide sequence of SEQ ID NO:5, SEQ ID NO:12, SEQ ID NO: 13, SEQ ID NO:20 or SEQ ID NO:21, or the complement or reverse complement of each of the sequences, or
(vi) the vector further comprises a third AAV ITR.

16. (canceled)

17. (canceled)

18. (canceled)

19. The AAV vector of claim 13, wherein the ITR is an AAV2 ITR and wherein the AAV2 ITR is truncated.

20. (canceled)

21. (canceled)

22. The AAV vector of claim 13, wherein the vector further comprises a third AAV ITR and wherein the third ITR is modified to inactivate the terminal resolution site.

23. The AAV vector of claim 1, wherein the vector further comprises a transcription control region operably linked with the nucleotide sequence encoding the PGRN polypeptide, variant.

24. The AAV vector of claim 23, wherein the transcription control region is tissue or cell type specific or wherein the transcription control region comprises a promoter and/or enhancer sequence that is brain tissue specific or neuron cell specific.

25. The AAV vector of claim 24, wherein the transcription control region is brain tissue specific or neuron specific or wherein the transcription control region is more transcriptionally active in CNS neurons than in hepatocytes.

26. (canceled)

27. (canceled)

28. The AAV vector of claim 24, wherein the transcription control region comprises a promoter and/or enhancer sequence, and wherein the promoter and/or enhancer sequence is derived from a synapsin gene.

29. (canceled)

30. The AAV vector of claim 1, wherein
(i) the vector further comprises a 5′ untranslated region (UTR) sequence,
(ii) the vector further comprises a transcription termination signal sequence,
(iii) the vector further comprises an intron sequence,
(iv) the vector further comprises a post-transcriptional regulatory element (PRE) sequence
(v) the vector further comprises a binding site for a microRNA (miRNA),
(vi) the vector further comprises a stuffer or filler nucleotide sequence of sufficient length such that the entire length of the AAV vector inclusive of ITRs is approximately 3.5 to 5.0 kilobases,
(vii) the vector comprises a first AAV ITR, a transcription control region operably linked with the nucleotide sequence encoding the PGRN polypeptide variant, a transcription termination signal sequence, and a second AAV ITR,
(viii) wherein the vector comprises in 5′ to 3′ order:
(a) a first AAV2 ITR,
(b) a promoter sequence from a synapsin gene
(c) a 5′ UTR sequence from a synapsin gene,
(d) a nucleotide sequence encoding a human PGRN polypeptide variant operably linked with the promoter sequence,
(e) a transcription termination signal sequence from a bovine growth hormone (bGH) gene,
(f) a sequence from a TBP gene intron, and
(g) a second AAV2 ITR.

31. (canceled)

32. (canceled)

33. (canceled)

34. The AAV vector of claim 30, wherein the vector further comprises a transcription termination signal sequence and wherein the transcription termination signal sequence is a polyadenylation (poly(A)) signal sequence.

35. (canceled)

36. (canceled)

37. (canceled)

38. (canceled)

39. The AAV vector of claim 30, wherein the vector further comprises a PRE sequence and wherein the PRE sequence is a woodchuck hepatitis virus PRE or a hepatitis B virus PRE sequence.

40. (canceled)

41. (canceled)

42. The AAV vector of claim 30, wherein the vector further comprises a stuffer or filler nucleotide sequence of sufficient length such that the entire length of the AAV vector inclusive of ITRs is approximately 3.5 to 5.0 kilobases and wherein the stuffer or filler nucleotide sequence is derived from a TATA binding protein (TBP) gene.

43. (canceled)

44. (canceled)

45. (canceled)

46. The AAV vector of claim 30, wherein the vector comprises in 5′ to 3′ order:
(a) a first AAV2 ITR,
(b) a promoter sequence from a synapsin gene
(c) a 5′ UTR sequence from a synapsin gene,
(d) a nucleotide sequence encoding a human PGRN polypeptide variant operably linked with the promoter sequence,
(e) a transcription termination signal sequence from a bovine growth hormone (bGH) gene,
(f) a sequence from a TBP gene intron, and
(g) a second AAV2 ITR, and
wherein the promoter sequence from a synapsin gene comprises, consists essentially of, or consists of the nucleotide sequence of SEQ ID NO:6; the 5′ UTR sequence from a synapsin gene comprises the nucleotide sequence of SEQ ID NO:7; the nucleotide sequence encoding the PGRN polypeptide variant comprises the nucleotide sequence of SEQ ID NO:8 or SEQ ID NO:15; the transcription termination signal sequence from a bovine growth hormone (bGH) gene comprises the nucleotide sequence of SEQ ID NO:9 or SEQ ID NO:10; and the sequence from a TBP gene intron comprises the nucleotide sequence of SEQ ID NO: 11.

47. The AAV vector of claim 46, wherein each of the first and second AAV2 ITRs comprises the nucleotide sequence of SEQ ID NO:5, SEQ ID NO:12, SEQ ID NO:13, SEQ ID NO: 20 or SEQ ID NO:21, or the complement or reverse complement of each of the sequences, or wherein the nucleotide sequence of the vector comprises the nucleotide sequence of SEQ ID NO:19 or the reverse complement thereof.

48. (canceled)

49. (canceled)

50. An AAV particle comprising an AAV capsid and the AAV vector of claim 1, wherein the vector is encapsidated by the capsid, wherein the AAV capsid is at least partially neurotropic .

51. (canceled)

52. (canceled)

53. (canceled)

54. (canceled)

55. (canceled)

56. A method of preventing or treating a disease or disorder in a human subject caused by a deficiency of human GRN polypeptide comprising administering to the subject an amount of the AAV vector of claim 1 effective to increase the amount of GNR polypeptide, or variant thereof, in at least one biofluid, tissue or cell of the subject.

57. (canceled)

58. (canceled)

59. (canceled)

60. (canceled)

61. (canceled)

62. (canceled)

63. (canceled)

64. (canceled)

65. (canceled)

66. A method of reducing the frequency or severity of at least one symptom or sign in a human subject caused by a deficiency of human GRN polypeptide comprising administering to the subject an amount of the AAV vector of claim 1 effective to reduce the frequency or severity of such symptom or sign.

67. (canceled)

68. (canceled)

69. (canceled)

70. (canceled)

71. (canceled)

72. (canceled)

73. (canceled)

74. A method of preventing or treating frontotemporal dementia in a human subject comprising administering to the subject a prophylactically or therapeutically effective amount of an AAV vector of claim 1 effective to prevent or treat frontotemporal dementia in the subject.

75. (canceled)

76. (canceled)

77. A DNA plasmid comprising the nucleotide sequence of the AAV vector of claim 1.

78. A host cell for AAV vector production comprising the DNA plasmid of claim 77.

79. (canceled)

80. (canceled)

81. (canceled)

82. (canceled)

83. A method of making an AAV vector, comprising: incubating the host cell of claim 78 under conditions sufficient to allow the production of AAV vectors, and purifying the AAV vectors produced thereby.

84. (canceled)
